SEQUENCE LISTING (28 sequences)

SEQ ID NO: 1 - DNA, 43 bp, Artificial Sequence
  synthetic primer
atgaattccatggcttctaactttactcagttcgttctcgtcg

SEQ ID NO: 2 - DNA, 47 bp, Artificial Sequence
  synthetic primer
tactgcagctattagtagatgccggagtttgctgcgattgctgaggg

SEQ ID NO: 3 - DNA, 393 bp, Artificial Sequence
  synthetic plant MS2 CP gene
atggcctccaacttcacccagttcgtgctcgtggacaacggcggcaccggcgacgtgaccgtcgccccaagcaacttcgccaacggggtcgccgagtggatcagctccaactcgcgctcccaggcctacaaggtgacctgcagcgtgcgccagtcctccgcgcagaaccgcaagtacaccatcaaggtcgaggtgccgaaggtggccacccagaccgtcggcggcgtggagctgccagtggccgcctggcgctcgtacctcaacatggagctgaccatcccaatcttcgccacgaactccgactgcgagctgatcgtgaaggccatgcaaggcctcctgaaggacggcaacccgatcccctccgccatcgccgccaactccggcatctactaa

SEQ ID NO: 4 - DNA, 100 bp, Artificial Sequence
  synthetic primer
atggcctccaacttcacccagttcgtgctcgtggacaacggcggcagcggcgacgtgaccgtcgccccaagcaacttcgccaacggggtcgccgagtgga

SEQ ID NO: 5 - DNA, 100 bp, Artificial Sequence
  synthetic primer
tgatggtgtacttgcggttctgcgcggaggactggcgcacgctgcaggtcaccttgtaggcctgggagcgcgagttggagctgatccactcggcgacccc

SEQ ID NO: 6 - DNA, 100 bp, Artificial Sequence
  synthetic primer
cgcaagtacaccatcaaggtcgaggtgccgaaggtggccacccagaccgtcggcggcgtggagctgccagtggccgcctggcgctcgtacctcaacatgg

SEQ ID NO: 7 - DNA, 100 bp, Artificial Sequence
  synthetic primer
ggttgccgtccttcaggaggccttgcatggccttcacgatcagctcgcagtcggagttcgtggcgaagattgggatggtcagctccatgttgaggtacga

SEQ ID NO: 8 - DNA, 57 bp, Artificial Sequence
  synthetic primer
ctgaaggacggcaacccgatcccctccgccatcgccgccaactccggcatctactaa

SEQ ID NO: 9 - DNA, 32 bp, Artificial Sequence
  synthetic primer
ccgggaattccttagtagatgccggagttggc

SEQ ID NO: 10 - DNA, 34 bp, Artificial Sequence
  synthetic primer
ggccggatccaccatggcctccaacttcacccag

SEQ ID NO: 11 - DNA, 76 bp, Artificial Sequence
  synthetic primer
ggccggatccatggccagcatgaccggcggccagcaaatgggcaccatggcctccaacttcacccagttcgtgctc

SEQ ID NO: 12 - DNA, 69 bp, Artificial Sequence
  synthetic primer
aaaaagatatccatggcttccatgactggtggacagcaaatgggaatggcttctaactttactcagttc

SEQ ID NO: 13 - DNA, 47 bp, Artificial Sequence; identical to SEQ ID NO: 2
  synthetic primer

SEQ ID NO: 14 - DNA, 33 bp, Artificial Sequence
  fusion junction
aactccggcatctacgccaacttcacccagttc

SEQ ID NO: 15 - DNA, 47 bp, Artificial Sequence
  synthetic primer
ataagctttaaattatctacttaaggaccatcaggccttaagaacac

SEQ ID NO: 16 - DNA, 54 bp, Artificial Sequence
  synthetic primer
taaagcttgatatcatatgttctcgagttttgtgttcttaaggcctgatggtcc

SEQ ID NO: 17 - DNA, 50 bp, Artificial Sequence
  synthetic primer
catttggagaggacgtactagtatataagctttttacaacaattaccaac

SEQ ID NO: 18 - DNA, 36 bp, Artificial Sequence
  synthetic primer
cggcggatccaccatggcaaaattagagactgttac

SEQ ID NO: 19 - DNA, 31 bp, Artificial Sequence
  synthetic primer
ggccgaattctcaatacgctgggttcagctg

SEQ ID NO: 20 - DNA, 402 bp, Artificial Sequence
  synthetic plant expression gene for Q-beta
atggccaagctcgagaccgtgaccctcggcaacatcggcaaggacggcaagcagaccctcgtgctcaacccgcgcggcgtgaacccgaccaacggcgtggcctccctctcccaggccggcgccgtgccggccctcgagaagcgcgtgaccgtgtccgtgtcccagccgtcccgcaaccgcaagaactacaaggtgcaggtgaacatccagaacccgaccgcctgcaccgccaacggctcctgcgacccgtccgtgacccgccaggcctacgccgacgtgaccttctccttcacccagtactccaccgacgaggagcgcgccttcgtgcgcaccgagctcgccgccctcctcgcctccccgctcctcatcgacgccatcgaccagctcaacccggcctactga

SEQ ID NO: 21 - DNA, 61 bp, Artificial Sequence
  synthetic Q-beta operator sequence
gcgctctagatgttctcgagttttgtgttggatcagtcttagacttgatcttcctaattgc

SEQ ID NO: 22 - DNA, 32 bp, Artificial Sequence
  synthetic primer
gttaacaagcttctgcaggtccgatgtgagac

SEQ ID NO: 23 - DNA, 76 bp, Artificial Sequence
  synthetic primer
gcgccccgggatgttctcgacttttgtgttcttaaggcctgatggtccttaagcccttggatcttatggtgtgttg

SEQ ID NO: 24 - DNA, 40 bp, Artificial Sequence
  synthetic primer
gcgcggatccaacaatggccccagttaaatcccaagaatc

SEQ ID NO: 25 - DNA, 35 bp, Artificial Sequence
  synthetic primer
gcgcgaattccttaagccaaggtggtcaagatatc

SEQ ID NO: 26 - DNA, 318 bp, Artificial Sequence
  synthetic Rpl32 enhanced plant expression gene seq.
atggccccggtgaagtcccaggagtccatcaaccagaagctcgccctcgtgatcaagtccggcaagtacaccctcggctacaagtccaccgtgaagtccctccgccagggcaagtccaagctcatcatcatcgccgccaacaccccggtgctccgcaagtccgagctcgagtactacgccatgctctccaagaccaaggtgtactacttccagggcggcaacaacgagctcggcaccgccgtgggcaagctcttccgcgtgggcgtggtgtccatcctcgaggccggcgactccgacatcctcaccaccctcgcctga

SEQ ID NO: 27 - DNA, 65 bp, Artificial Sequence
  synthetic primer
cgcgtctagatcgacgatctctgcgtataaaacactcccgtctcccttactagtacgtcctctcc

SEQ ID NO: 28 - DNA, 23 bp, Artificial Sequence
  correct orientation of operator
cttaaggaccatcaggccttaag

(19) United States
(12) Patent Application Publication
(10) Pub. No.: US 2002/0062499 A1
(43) Pub. Date: May 23, 2002

(54) Methods for translational repression of gene expression in plants

(76) Inventors: Timothy W. Conner, Wildwood, MO (US); Bradon J. Fabbri, Chesterfield, MO (US); Jintai Huang, Wildwood, MO (US)

Correspondence Address: MONSANTO COMPANY, 800 N. LINDBERGH BLVD., ATTENTION: G.P. WUELLNER, IP PARALEGAL, (E2NA), ST. LOUIS, MO, 63167, US

(21) Appl. No.: 09/851,190

(22) Filed: May 8, 2001

Related U.S. Application Data
(60) Provisional application No. 60/203,060, filed on May 8, 2000.

(51) Int. Cl. C12N 15/82; C12N 15/33; A01H 5/00

(52) U.S. Cl. 800/287; 800/300; 800/271; 800/303; 800/274; 800/288; 536/23.72

(57) ABSTRACT

The methods and materials disclosed herein are directed to the control of gene expression in plants by means of translational repression. RNA-binding proteins binding specifically to operator sequences positioned in the 5′ untranslated region of an MRNA reduce translation. Such translation repression systems are useful, for example, for reducing expression of an herbicide-tolerance gene in reproductive tissues of a plant that retains vegetative tolerance. Application of the herbicide renders the plant male- or female-sterile.

This application claims priority to U.S. provisional application No. 60/203,060, filed May 8, 2000, herein incorporated by reference in its entirety.

### FIELD OF THE INVENTION

The present invention relates generally to the genetic engineering of plants for control of transgene expression, and specifically to methods and compositions for modulating the translation of a transgene in a plant.

### BACKGROUND OF THE INVENTION

Much effort has been directed at controlling transgene expression in plants by selecting or modifying promoters to make it possible to induce or repress transgene expression at desired times. There are many opportunities for modification of plant phenotypes and the production of valuable products in plants if a higher degree of control of transgene expression can be achieved. Many potential environmental issues such as herbicide resistance in wild related plant species, insecticidal effects on non-target organisms from pollen or crop residue, potential for weediness of the engineered crop, and insect resistance management can be addressed by tightly controlled transgene expression. In addition, inducible or repressible promoters are useful, for example, in conferring late-season herbicide sensitivity to permit the use of herbicides as a harvest aid; in specifically inhibiting transgene expression in the final fruit or seed product to improve food and feed safety; in affecting plant growth and development or resistance or tolerance to pathogens or environmental stresses; in permitting tissue-specific gene excision; and in production of hybrid plant varieties.

Translational Repression.

One mechanism for control of gene expression in both prokaryotes and eukaryotes is translational regulation, which results from a specific interaction between cis-acting sequences (RNA-binding protein site) contained in mRNA and trans-acting proteins (RNA-binding protein) that bind specifically to these cis-acting sequences. In most cases, these cis-acting sequences or translation operators are found in the untranslated region (UTR) at the 5′ or 3′ end of the mRNA transcripts (Jackson, Cell 74:9-14 (1993); Melefors, Bioessays 15:85-90 (1993); Sonenberg, Curr. Opin. Genet. Dev. 4:310-315 (1994); van der Velden, Int. J. Biochem. Cell Biol. 31:87-106 (1999)).

The expression of numerous prokaryotic genes is translationally regulated by the specific interaction between repressor proteins and RNA binding sequences. In many cases, these repressor proteins directly or indirectly occlude the Shine-Dalgarno sequence or the initiation codon (Gold, Annu. Rev. Biochem. 57:199-233 (1988); McCarthy et al., Trends Genet. 6:78-85 (1990); Witherell, Progr. Nucl. Acid Res. Mol. Biol. 40:185-220 (1991)). A well-studied example of translational repression in prokaryotes is the repression of translation of the viral replicase gene by the coat protein (CP) of the RNA bacteriophages. The RNA bacteriophages and those specifically known as RNA coliphages infect E. coli, are taxonomically related as members of the Leviviridae family and further divided into four main groups. These groups are represented by MS2 (group I), GA (group II), Qβ (group III), and SP (group IV). Other RNA coliphages include R17, f2, β, fr, f4, JP34, and ID2. RNA bacteriophages that infected bacteria other than E. coli include: 7S, PP7, Cb5, Cb12r, Cb8r, Cb23r and PRR1 (Fraenkel-Conrat et al., The Bacteriophages, Vol 1., Plenum Press NY (1988)).

Translational repression of the MS2 RNA bacteriophage replicase gene results from binding of MS2 coat protein (CP) to a RNA-binding protein operator (op) nucleic acid sequence in the 5′ UTR of the viral replicase mRNA (Peabody, J. Biol. Chem. 265:5684-5689, 1990; Uhlenbeck et al., J. Biomol. Struct. Dyn. 1:539-552 (1983)). The MS2 CP and its cognate operator are necessary and sufficient to confer translation repression of an unrelated mRNA carrying the op in its 5′ UTR (Stripecke et al., Nucl. Acids Res. 20:5555-5564 (1992)). The coat proteins of other coliphages, including the group II phage GA and group III phage Qβ, are likely to also inhibit translation of their replicase MRNA by a similar mechanism (Gott et al., Nucl. Acids Res. 19:6499-6503 (1991); Lim et al., J. Biol. Chem. 271:31839-31845 (1996)).

The MS2 CP/op system also confers translational repression to heterologous mRNA in vivo in yeast and mammalian cells (Berkhout et al., Nucl. Acids Res. 18:6903-6907 (1990); Stripecke et al., Mol. Cell. Biol. 14:5898-5909 (1994)). MS2 CP mutants have been identified that have higher binding affinity or enhanced stability (Lim et al., Nucl. Acids Res. 22:3748-3752 (1994)). MS2 op variants also have been identified. One particular variant, the −5C mutant, has a 150-fold increase in Ka value (Romaniuk et al., Biochem. 26:1563-1568 (1987)). When tested in an in vitro translation system, only the −5C variant showed significant translational repression of the reporter mRNA, whereas the wild type op was inactive even if the MS2 CP was in 80,000-fold excess (Stripecke et al., Nucl. Acids Res. 20:5555-5564 (1992)).

In higher organisms, translational regulation of gene expression plays a pivotal role in many important biological process such as embryogenesis and spermatogenesis (Hershey, Annu. Rev. Biochem. 60:715-755 (1991); McCarthy, Microbiol. Mol. Biol. Rev. 62:1492-1553 (1998); Venables et al., Curr. Opin. Genet. Dev. 9:346-354 (1999)). Regulated translational repression in embryogenesis depends on the specific binding of trans-acting protein factors to sequences in the 3′ UTR (Seydoux, Curr. Opin. Genet. Dev. 6:555-561 (1996)). Lox-BP also binds to the 3′ UTR of erythroid 15-lipoxygenase mRNA and represses its translation (Ostareck-Lederer, EMBO J. 13:1476-1481 (1994)). IRP/IRE (Gray et al., Eur. J. Biochem, 18:657-667 (1993), Paraskeva et al., Mol. Cell. Biol. 19:807-816 (1999)); U1A spliceosomal protein and binding sequence (Oubridge et al., Nature 372:432-438 (1994)); T4 regA protein and binding sequence (Kang et al., Science 268:1170-1173 (1995)); the Drosophila PUM protein/NRE binding sequence (Zamore et al., Biochemistry 38:596-604 (1999)); and ribosomal proteins, tRNA synthetases and all RNA-binding proteins listed in Arnez et al., Quarterly Rev. Biophysics 30:195-240 (1997) are all examples of RNA-binding proteins. Translational regulation also represents an important regulatory mechanism that controls gene expression in higher plants (Cohen, Curr. Opin. Biotech. 8:189-194 (1997)).

Male- and Female-Sterile Plants for Hybrid Seed Production.

Hybrid varieties of crop plants have had a huge impact on worldwide food production and have great potential for providing higher yielding crop plants for the world's growing population. Plants produced from such hybrid seed can have substantially superior agronomic performance characteristics, including, for example, plant size, grain yield, disease resistance, herbicide tolerance, and improved climatic adaptation. Hybrid seed production requires that cross-pollination predominates over self-pollination. Efficient production of hybrid plants has been limited to crops where the male and female parts can be most easily physically separated such as in mechanical detasseling of corn (Sprague et al., Corn and Corn Improvement 3^{rd} Edition, Am. Soc. Agron. Publ (1988)), or where chemical gametocides are available, such as for wheat (Genesis®) (Fichet, Assoc. Nat. Protec. Plantes, Paris France [conference paper], pp. 61-68 (1996)). A major limitation in the production of hybrid seed for most crop species is the lack of simple, reliable and economical methods of generating male sterility while leaving female gametes intact and accessible for pollination by a suitable pollen donor. Most crop species are self-pollinated or the male and female parts are in the same flower and not easily separated. There is a need for a broadly applicable genetic and/or chemical tool for producing hybrid varieties cheaply and efficiently from any plant species.

An effective chemical male gametocide is a compound that when applied to a plant at an appropriate developmental stage or before sexual maturity is capable of killing or effectively terminating the development of a plant's male gametes while leaving the plant's female gametes, or at least a significant proportion of them, capable of undergoing cross-pollination. For an effective male gametocide, it is desired that the application level at which male gametes are destroyed is significantly lower than that required to destroy the female gametes. Currently available chemical hybridizing agents (CHA) that are primarily directed at inhibiting male gametes also often negatively affect female fertility. Careful dosing in the field is necessary for good hybrid seed production. This is often problematic due to weather conditions and the limited window of application at the plant developmental stage most likely to result in a high level of male sterility while maintaining female fertility. Selective female gametocides that would prevent seed production by the male donor parent have not been extensively developed. Thus physical separation of the male donor parent and the CHA-treated female parent is required. A method for application of a single gametocide that would produce both a male-sterile, female-fertile parent and a male-fertile, female-sterile parent in the same hybrid production field would be highly desirable.

In addition, many chemical gametocides that have shown good selectivity have toxicological issues or other environmental issues that limit the use of these compounds for production of commercial levels of hybrid seeds. Thus, there is a need for methods to improve the selectivity and the environmental safety of gametocides for production of hybrid seed.

Several naturally occurring genetic mechanisms that confer male sterility have been exploited for the production of hybrid seed in some plant species. In many instances, male sterility results from the developmental arrest of the pollen or the anther tissues that nourish the developing pollen grains and release the mature pollen with the correct timing. Hybridization strategies using cytoplasmic male sterile (CMS) systems have been successfully employed in some plant species. A disadvantage of this approach is that it requires three distinct lines to produce a single crossed hybrid: the male-sterile line (female parent), a maintainer line that is isogenic to the male-sterile line but contains fully functional mitochondria, and the male parent line. Many CMS types have unfavorable characteristics that restrict their use, including undesirable linked or pleiotropic characteristics such as disease susceptibility, breakdown of sterility, and inconsistent or complexly inherited fertility restoration. Furthermore, CMS is unavailable in many important crop species, and full sterility due to the CMS cytoplasm is not always exhibited in different nuclear genetic backgrounds within a species. In those species for which a CMS is widely used in hybrid seed production, there can be an unsafe dependence on a single sterile cytoplasm (Williams et al., Plant Breeding Rev. 10:23-52 (1992)). The southern corn leafblight caused by Helminthosporium maydis, Race T, which severely attacked all maize hybrids with cytoplasmic male-sterile T cytoplasm, demonstrates the vulnerability of a hybrid seed industry that relies too heavily on a single source of a male-sterile cytoplasm.

Genetic engineering has the potential to make a significant contribution to agricultural productivity by providing economical alternatives to the methods that are currently used for producing hybrid seed (Williams, Trends Biotech. 13:344-349 (1995)). For example, selective expression of genes encoding cytotoxic proteins can allow for the production of a uniform population of male-sterile plants. In one example, barnase was expressed by a tobacco tapetal-specific promoter in anther tapetal cells and caused male sterility. Fertility could be restored in progeny by crossing with a plant containing a tapetal-specific promoter driving the expression of the barstar gene (Marini et al., Nature 357:384-387 (1992), Zhan et al. Sexual Plant Reprod. 9:35-43 (1996)). The combination of barnase and barstar has been used to ablate specific anther cell types useful for the identification of cell types necessary for the maturation of anthers and pollen release (Goldberg et al., Philos. Trans. Roy. Soc. (London) 2:603-618 (1995); Beals et al., Plant Cell 9:1527-1545 (1997)). As another example, the expression of a DAM-methylase is cytotoxic to pollen formation when expressed in anthers by an anther-specific promoter (WO 9617945).

The expression of RNA that is complementary (antisense) to an endogenous gene that is critical for proper growth and development of anthers or pollen has been disclosed as a method for generating male sterility, such as by inhibiting expression of an essential amino acid by antisense to an aspartokinase gene in pollen or tapetal cells (EP 93109226), or the QM gene in maize (U.S. Pat. No. 5,583,210). Also the expression of metabolically active enzymes such as an ATPase (Zabaleta et al., Proc. Natl. Acad. Sci. USA 93:1259-1263 (1996)) in pollen or associated cells can result in male sterility. The expression of a nucleic acid sequence that is antisense to a herbicide resistance gene by a male-specific tissue promoter has been shown to confer sensitivity to the toxic effects of the herbicide (U.S. Pat. No. 5,728,926 and WO 9946396).

Glyphosate as a Gametocide.

N-phosphonomethylglycine, also known as glyphosate, is a well-known herbicide that has activity on a broad spectrum of plant species. Glyphosate is the active ingredient of Roundup® (Monsanto Co.), a safe herbicide having a desirably short half life in the environment. When applied to a plant surface, glyphosate moves systemically through the plant. Glyphosate is phytotoxic due to its inhibition of the shikimic acid pathway, which provides a precursor for the synthesis of aromatic amino acids. Specifically, glyphosate effects the conversion of phosphoenolpyruvate and 3-phosphoshikimic acid to 5-enolpyruvyl-3-phosphoshikimic acid by inhibiting the enzyme 5-enolpyruvyl-3-phosphoshikimate synthase (EPSPS).

Through the methods of plant genetic engineering, it is possible to produce glyphosate-tolerant plants by inserting into the plant genome a DNA molecule that causes the production of higher levels of wild-type EPSPS. Glyphosate tolerance can also be achieved by the expression of EPSPS variants that have lower affinity for glyphosate and therefore retain their catalytic activity in the presence of glyphosate (U.S. Pat. No. 5,633,435). Enzymes that degrade glyphosate in plant tissues (U.S. Pat. No. 5,463,175) are also capable of conferring cellular tolerance to glyphosate. Such genes allow for the production of transgenic crops that are tolerant to glyphosate, thereby allowing glyphosate to be used for effective weed control with minimal concern of crop damage. For example, glyphosate tolerance has been genetically engineered into corn (U.S. Pat. No. 5,554,798), wheat (Zhou et al. Plant Cell Rep. 15:159-163 (1995)), soybean (WO 9200377) and canola (WO 9204449).

The use of glyphosate and related compounds as chemical gametocides has been described. See for example U.S. Pat. No. 4,735,649 in which it is disclosed that under optimal conditions compounds related to glyphosate can kill about 95% of male gametes in plants not genetically engineered for glyphosate tolerance, while leaving about 40-60% of the female gametes capable of fertilization. Use of glyphosate as a selective female gametocide has not been previously exploited in a hybrid production.

### SUMMARY OF THE INVENTION

The present invention herein provides compositions and methods for modulating gene expression by translational repression of gene expression in plants.

According to one aspect of the invention, recombinant nucleic acid constructs are provided in which translation of a gene of interest is modulated by binding of an RNA-binding protein to one or more operator sequences that are located in the 5′ untranslated region of the gene such that the operator sequence(s) are included in the MRNA produced by transcription of the gene, i.e., with respect to the DNA sequence transcribed to produce the mRNA transcript, 3′ to the 5′ end of the transcript. Such nucleic acid constructs comprise a transcriptional unit that comprises: (a) a 5′ untranslated region comprising a first promoter that is functional in a cell of a plant and at least one operator for binding of an RNA-binding protein, (b) a polypeptide-encoding DNA sequence that is expressed under the control of the promoter, and (c) a first 3′ non-translated region comprising a polyadenylation site operably linked to the polypeptide-encoding DNA sequence. Transcription of the transcriptional unit in a cell of the plant produces an mRNA for the gene of interest comprising the operator(s), and binding of the RNA-binding protein to the operator(s) modulates translation of the mRNA.

According to one embodiment of the invention, the operator is located between 0 and 37 nucleotides, inclusive, 3′ to the 5′ end of the mRNA. According to another embodiment, the RNA-binding protein operator is located between 4 and 19 nucleotides, inclusive, 3′ to the 5′ end of the mRNA produced by transcribing the target gene.

According to another embodiment of the invention, the 5′ untranslated region of the nucleic acid construct comprises multiple operators for binding of an RNA-binding protein, which may optionally be in tandem array. Operators in tandem array may optionally be separated by a spacer one or more nucleotides in length, including, for example introns.

To effect translational modulation of the expression of a gene of interest, the RNA-binding protein must be expressed in a cell in which mRNA corresponding to the target gene of interest (and including an operator in its 5′ untranslated region) is likewise present. The DNA sequence encoding the RNA-binding protein may be part of the same nucleic acid construct as the target gene of interest or as part of a second nucleic acid construct. According to another aspect of the invention, therefore, the nucleic acid construct described above further comprises a second transcriptional unit that comprises (a) a second promoter that is functional in the cell of the plant; (b) a DNA sequence that encodes the RNA-binding protein; and (c) a second 3′ non-translated region comprising a polyadenylation site operably linked to the DNA sequence that encodes the RNA-binding protein.

According to another embodiment of the invention, the DNA sequence that encodes the RNA-binding protein is modified for enhanced plant expression, such as by replacing codons of the protein-coding portion(s) of the DNA sequence with codons that are preferred by the plant (i.e., are found in protein-coding sequences of the plant at a higher frequency) without altering the amino acid sequence of the RNA-binding protein.

According to another embodiment of the invention, the DNA sequence that encodes the RNA-binding protein encodes an RNA-binding protein dimer.

The promoter driving expression of the targeted gene of interest may have the same expression pattern as the promoter driving expression of the RNA-binding protein, or it may have a different expression pattern such that translational repression of the target gene occurs only in a particular cell or tissue of the plant, at a particular developmental stage, etc., corresponding to the expression pattern of the RNA-binding protein. According to one aspect of the invention, the promoter driving expression of the targeted gene of interest is a constitutive promoter.

According to another aspect of the invention, with respect to a nucleic acid construct that includes transcriptional units for both the targeted gene of interest and the RNA-binding protein, the promoter driving expression of the RNA-binding protein is expressed selectively in a tissue of the plant such that translation of the polypeptide-encoding DNA sequence is modulated in that tissue. A number of RNA-binding proteins and their corresponding operator sequences are described herein, including but not limited to various RNA bacteriophage coat proteins, including coat proteins from MS2 and Qβ phage, yeast ribosomal proteins (e.g., the RPL32 RNA-binding protein), etc.

For example, for production of hybrid seeds it may be desirable to modulate expression of a gene of interest in a male or female reproductive tissue by preferentially expressing the RNA-binding protein in that tissue, thereby affecting male or female fertility of the plant, respectively. In one approach to modulating expression of the gene of interest in a male tissue, for example, the promoter used to drive expression of the RNA-binding protein may be a promoter that is preferentially expressed in a male tissue of the plant. According to one embodiment of the invention, the polypeptide-encoding DNA sequence is a gene required for male fertility, such that expression of the RNA-binding polypeptide in the male reproductive tissue reduces translation of the polypeptide-encoding DNA sequence and thereby causes male sterility.

Similarly, it may be desirable to modulate expression of a gene of interest in a female reproductive tissue. In one approach, the promoter used to drive expression of the RNA-binding protein may be any promoter that is preferentially expressed in a female tissue of the plant. According to one embodiment of the invention, the polypeptide-encoding DNA sequence is a gene required for female fertility, such that expression of the RNA-binding polypeptide in the female reproductive tissue reduces translation of the polypeptide-encoding DNA sequence and thereby causes female sterility.

According to another aspect of the invention, a nucleic acid construct as described above is provided in which the promoter driving expression of the polypeptide-encoding DNA sequence is a constitutive promoter and the polypeptide-encoding DNA sequence encodes a polypeptide that confers tolerance to a herbicide to the plant. The promoter driving expression of the RNA-binding protein causes the RNA-binding protein to be selectively expressed in a reproductive tissue of the plant. As a result, a plant transformed with the nucleic acid construct is vegetatively tolerant to an application of the herbicide and the reproductive tissue is sensitive to the application of the herbicide. Such a system can be used to confer vegetative tolerance and reproductive (male or female) sensitivity to such herbicides as glyphosate. According to one embodiment, vegetative tolerance and male or female sensitivity is conferred through the use of target DNA sequences that encode a glyphosate-resistant EPSP synthase or a glyphosate-degrading enzyme (e.g., glyphosate oxidoreductase), for example. According to one hybrid-breeding system, an otherwise herbicide-tolerant female parent plant is rendered male sterile by application of a herbicide, and the male parent plant is likewise rendered female sterile by application of the herbicide, permitting the male and female parents to be interplanted. Following herbicide application, pollen shed by the male-fertile, female-sterile male parent plant fertilizes the male-sterile, female-fertile female parent plant in the hybrid production field. In interplanting schemes, the ratio of male-fertile, female-sterile plants to male-sterile, female-fertile plants can be relatively low, thereby increasing yields of the hybrid. For wheat, a ratio of about 1:5 to about 1:20 is practicable, while for corn, a ratio of about 1:5 to about 1:10 is practicable.

According to another aspect of the invention, plants are provided that comprise one or more of the nucleic acid constructs described above.

According to another aspect of the invention, related methods for controlling translation of a polypeptide-encoding DNA sequence in a plant are provided. According to one embodiment of the invention, these methods include the steps of: (1) providing a plant comprising a recombinant nucleic acid construct that comprises: (1) a first transcriptional unit comprising a 5′ untranslated region comprising a first promoter that is functional in the plant and at least one operator for binding of an RNA-binding protein, a polypeptide-encoding DNA sequence that is expressed under the control of the promoter, and a first 3′ non-translated region comprising a polyadenylation site operably linked to the polypeptide-encoding DNA sequence, and a second transcriptional unit comprising a second promoter that is functional in the cell of the plant, a DNA sequence that encodes the RNA-binding protein, and a second 3′ non-translated region comprising a polyadenylation site operably linked to the DNA sequence that encodes the RNA-binding protein; (2) transcribing the first transcriptional unit in the plant cell to produce an mRNA comprising said at least one operator sequence, and (3) transcribing and translating the second transcriptional unit to produce the RNA-binding protein in a cell of the plant, wherein binding of the RNA-binding protein to said at least one operator modulates translation of the mRNA.

Similarly, methods are provided for producing hybrid seed comprising the steps of: (1) providing a pollen-producing male parent and a male-sterile female parent, the female parent comprising: a first transcriptional unit comprising a 5′ untranslated region comprising a first promoter that is functional in the female parent and at least one operator for binding of an RNA-binding protein, a polypeptide-encoding DNA sequence that is expressed under the control of the promoter, and a first 3′ non-translated region comprising a polyadenylation site operably linked to the polypeptide-encoding DNA sequence, and a second transcriptional unit comprising a second promoter that is expressed in a male reproductive tissue of the female parent, a DNA sequence that encodes the RNA-binding protein, and a second 3′ non-translated region comprising a polyadenylation site operably linked to the DNA sequence that encodes the RNA-binding protein; wherein expression of the RNA-binding polypeptide in the male reproductive tissue reduces translation of the polypeptide-encoding DNA sequence and thereby causes male sterility; and (2) fertilizing the female parent with pollen from the male parent, thereby producing hybrid seed.

According to another embodiment of the invention, methods are provided for producing a hybrid seed comprising: (1) providing a pollen-producing male parent and a male-sterile female parent, the female parent comprising: a first transcriptional unit comprising a 5′ untranslated region comprising a first promoter that is functional in the female parent and at least one operator for binding of an RNA-binding protein, a herbicide-tolerance gene that is transcribed under the control of the promoter, and a first 3′ non-translated region comprising a polyadenylation site operably linked to the polypeptide-encoding DNA sequence, and a second transcriptional unit comprising a second promoter that is expressed in a male reproductive tissue of the female parent, a DNA sequence that encodes the RNA-binding protein, and a second 3′ non-translated region comprising a polyadenylation site operably linked to the DNA sequence that encodes the RNA-binding protein; wherein expression of the RNA-binding polypeptide in the male reproductive tissue reduces translation of the herbicide-tolerance gene in the male reproductive tissue; (2) applying a herbicide to the female parent, thereby rendering the female parent male sterile; and (3) fertilizing the female parent with pollen from the male parent, thereby producing hybrid seed. Optionally, the male parent may comprise a third transcriptional unit comprising a 5′ untranslated region comprising a third promoter that is functional in the male parent and at least one operator for binding of an RNA-binding protein, a herbicide-tolerance gene that is expressed under the control of the third promoter, and a first 3′ non-translated region comprising a polyadenylation site operably linked to the herbicide-tolerance gene, and a fourth transcriptional unit comprising a fourth promoter that is expressed in a female reproductive tissue of the male parent, a DNA sequence that encodes the RNA-binding protein, and a fourth 3′ non-translated region comprising a polyadenylation site operably linked to the DNA sequence that encodes the RNA-binding protein; wherein expression of the RNA-binding polypeptide in the female reproductive tissue reduces translation of the herbicide-tolerance gene in the female reproductive tissue, the method further comprising the step of applying the herbicide to the male parent, thereby rendering the male parent female sterile.

As used herein, the term “comprise” can be used interchangeably with the phrase “includes, but is not limited to.”

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Plasmid map of pMON42176

FIG. 2. Plasmid map of pMON42177

FIG. 3. Plasmid map of pMON42180

FIG. 4. Plasmid map of pMON52035

FIG. 5. Plasmid map of pMON42409

FIG. 6. Plasmid map of pMON42426

FIG. 7. Plasmid map of pMON42916

FIG. 8. Plasmid map of pMON42178

FIG. 9. Plasmid map of pMON42410

FIG. 10. Plasmid map of pMON30510

FIG. 11. Plasmid map of pMON30511

FIG. 12. Plasmid map of pMON30512

FIG. 13. Plasmid map of pMON42440

FIG. 14. Plasmid map of pMON42928

FIG. 15. Plasmid map of pMON42445

FIG. 16. Plasmid map of pMON42420

FIG. 17. Plasmid map of pMON42452

FIG. 18. Plasmid map of pMON52012

FIG. 19. Plasmid map of pMON42434

FIG. 20. Plasmid map of pMON42435

FIG. 21. Plasmid map of pMON42919

FIG. 22. Plasmid map of pMON42934

FIG. 23. Plasmid map of pMON42935

FIG. 24. Plasmid map of pMON42985

FIG. 25. Plasmid map of pMON42914

FIG. 26. Plasmid map of pMON52008

FIG. 27. Plasmid map of pMON52001

FIG. 28. Plasmid map of pMON42438

FIG. 29. Plasmid map of pMON42439

FIG. 30. Plasmid map of pMON42945

FIG. 31. Plasmid map of pMON42987

### SEOUENCE LISTINGS

SEQ ID NO:1—Forward Primer MS2 CP

SEQ ID NO:2—Reverse Primer MS2CP

SEQ ID NO:3—MS2 CP enhanced plant expression sequence

SEQ ID NO:4—synms2pr1

SEQ ID NO:5—synms2pr2

SEQ ID NO:6—synms2pr3

SEQ ID NO:7—synms2pr4

SEQ ID NO:8—synms2pr5

SEQ ID NO:9—synms2pr6

SEQ ID NO:10—synms2pr7

SEQ ID NO:11—synms2pr8

SEQ ID NO:12—Forward primer T7

SEQ ID NO:13—Reverse primer T7

SEQ ID NO:14—MS2 CP fusion junction

SEQ ID NO:15—Forward primer MS2 op

SEQ ID NO:16—Reverse primer MS2 op

SEQ ID NO:17—Mutagenesis primer

SEQ ID NO:18—JHQBU

SEQ ID NO:19—JHQBL2

SEQ ID NO:20—Qβ enhanced plant expression gene sequence

SEQ ID NO:21—QBopL

SEQ ID NO:22—35S-15

SEQ ID NO:23—MS2opSmaILI

SEQ ID NO:24—JHRPL32U

SEQ ID NO:25—JHRPL32L

SEQ ID NO:26—Rpl32 enhanced plant expression gene sequence

SEQ ID NO:27—JHMINI32L

SEQ ID NO:28—correct orientation of operator

### DETAILED DESCRIPTION OF THE INVENTION

Herein we describe and exemplify compositions and methods for modulating gene expression by translational repression of transgenes in plants, such as, for example, genes conferring tolerance to an herbicide or antibiotic, insecticidal protein genes, genes that affect plant growth, metabolism or development, and genes encoding pharmaceutical proteins, for example. Such compositions and methods may be used with respect to any plant that can be genetically modified by biotechnology.

Translation of a gene of interest can be reduced or eliminated by binding of an RNA-binding protein to one or more operator sequences in the 5′ UTR of the mRNA transcript. The bound RNA-binding protein interferes with translation, likely by preventing ribosome assembly or blocking the movement of the ribosome along the transcript from 5′ to 3′. Such RNA-binding proteins may be multimeric, e.g., dimers of a particular RNA-binding protein. With respect to the DNA sequence encoding the mRNA transcript, the operator(s) is preferably located between 0 and 37 nucleotides, inclusive, more preferably between 4 and 19 nucleotides, inclusive, 3′ to the 5′ end of the transcript encoded by the DNA sequence. Optionally, multiple operators may be employed. For example, operators may be employed in tandem array (with the operator sequences immediately adjacent one another or spaced apart a selected distance). A DNA sequence encoding the RNA-binding protein may be part of the same DNA construct as the target gene or part of a second DNA construct. To modulate translation of a target gene, the RNA-binding protein must be expressed in a cell in which the target gene is also expressed. The promoter driving expression of the targeted gene of interest may have the same expression pattern as the promoter driving expression of the RNA-binding protein, in which case translation of the target gene will likely be modulated in all cells in which the target gene is expressed. Alternatively, the RNA-binding protein may have a different expression pattern, in which case translation of the target gene will be modulated only in a particular cell or tissue of the plant, at a particular developmental stage, etc., corresponding to the expression pattern of the RNA-binding protein.

For example, for production of hybrid seeds, it may be desirable to repress expression of a gene of interest in a male or female reproductive tissue by preferentially expressing the RNA-binding protein in that tissue, thereby affecting male or female fertility of the plant, respectively. The gene whose translation is repressed by the RNA-binding protein may be expressed under the control of a constitutive promoter or a non-constitutive promoter that causes expression in the same cell as the RNA-binding protein. In order to produce male-sterile plants, for example, the promoter selected to drive expression of the RNA-binding protein may be preferentially expressed in the male tissue. According to one embodiment of the invention, the polypeptide-encoding DNA sequence is a gene required for male fertility, such that expression of the RNA-binding polypeptide in the male reproductive tissue reduces translation of the polypeptide-encoding DNA sequence and thereby causes male sterility.

Similarly, it may be desirable to modulate expression of a gene of interest in a female reproductive tissue. In one approach, the promoter used to drive expression of the RNA-binding protein may be any promoter that is preferentially expressed in a female tissue of the plant. According to one embodiment of the invention, the polypeptide-encoding DNA sequence is a gene required for female fertility, such that expression of the RNA-binding polypeptide in the female reproductive tissue reduces translation of the polypeptide-encoding DNA sequence and thereby causes female sterility.

The compositions and methods of the present invention are also useful for modulating expression of a number of other genes of interest that are expressed in plants as transgenes, including, but not limited to, herbicide resistance genes; insecticidal protein genes from Bacillus species and other bacteria, fungi, and plants; antibiotic protein genes from viruses, bacteria, fungi, plants and animals; genes affecting plant growth and development, such as genes involved in plant hormone biosynthesis or degradation, vitamin biosynthesis, and cellular architecture; and pharmaceutical protein genes. For example, it may be desirable to reduce or eliminate expression of a transgene in a particular cell, tissue, or organ but not in other parts of a plant in which the transgene is expressed.

Definitions and Methods

The following definitions and methods are provided to better define the present invention and to guide those of ordinary skill in the art in the practice of the present invention. Unless otherwise noted, terms are to be understood according to conventional usage by those of ordinary skill in the relevant art. Definitions of common terms in molecular biology may also be found in Rieger et al., Glossary of Genetics: Classical and Molecular, 5th edition, Springer-Verlag: New York (1991); and Lewin, Genes V, Oxford University Press: New York (1994). The nomenclature for DNA bases as set forth at 37 CFR § 1.822 is used. The standard one- and three-letter nomenclature for amino acid residues is used.

Abbreviations for nucleotide bases in nucleic acid codes as used herein are: A=adenosine; C=cytosine; G=guanosine; T=thymidine. Codes used for synthesis of oligonucleotides as used herein are: N=equimolar A, C, G, and T; I=deoxyinosine; K=equimolar G and T; R=equimolar A and G; S=equimolar C and G; W=equimolar A and T; Y=equimolar C and T.

“CP4”.

“aroA:CP4 EPSPS” and “CP4 EPSPS” and “CP4” refer to the EPSP synthase gene or protein purified from Agrobacterium tumefaciens (AGRTU) strain CP4 that when expressed in plants confers tolerance to the herbicide glyphosate. The gene sequence maybe native or modified for enhanced expression in plants.

“Fragment”.

A “fragment” of a particular nucleic acid is a portion of the nucleic acid that is less than full-length and comprises at least a minimum length capable of hybridizing specifically with a target nucleic acid under stringent hybridization conditions. The length of such a fragment is preferably at least 15 nucleotides, more preferably at least 20 nucleotides, and most preferably at least 30 nucleotides of a native nucleic acid sequence.

“Homolog”.

A “homolog” of a gene of one species that encodes an RNA-binding protein is a nucleic acid sequence to which a probe or primer derived from the gene that binds under at least moderately stringent hybridization conditions to a nucleic acid sequence of a second species and that also encodes an RNA-binding protein.

“Isolated”.

An “isolated” nucleic acid is substantially separated or purified away from other nucleic acid sequences in the cell of the organism in which the nucleic acid naturally occurs, i.e., other chromosomal and extrachromosomal DNA and RNA, by conventional nucleic acid-purification methods. The term also embraces recombinant nucleic acids and chemically synthesized nucleic acids.

“Chimeric”.

The term “chimeric” refers to the product of the fusion of portions of two different nucleic acids or proteins.

Glyphosate”. For the purposes of the present invention, the term “glyphosate” includes any herbicidally active form of N-phosphonomethylglycine (including any salt thereof) and other forms that result in the production of the glyphosate anion in plants. Glyphosate is the active ingredient of Roundup® and Roundup Ultra® herbicide formulations, for example (Monsanto Company, St. Louis, Mo.). Treatments with “glyphosate” refer to treatments with the Roundup® or Roundup Ultra® herbicide formulation, unless otherwise stated. Plant transformation and regeneration in tissue culture use glyphosate or salts of glyphosate. Whole plant assays use formulated Roundup®.

“Glyphosate-tolerance Gene”.

The term “glyphosate-tolerance gene” refers to any gene that, when expressed as a transgene in a plant, confers the ability to tolerate levels of the herbicide glyphosate that would otherwise damage or kill the plant. Any glyphosate tolerance gene known to the skilled individual is suitable for use in the practice of the present invention. Glyphosate inhibits the shikimic acid pathway that leads to the biosynthesis of aromatic compounds including amino acids, plant hormones and vitamins. Specifically, glyphosate inhibits the enzyme 5-enolpyruvylshikimate-3-phosphate synthase (EPSPS). A variety of native and variant EPSPS enzymes have been expressed in transgenic plants in order to confer glyphosate tolerance (Singh et al., In “Biosynthesis and Molecular Regulation of Amino Acids in Plants”, Amer. Soc. Plant Phys. (1992)), any of which can be used in the invention. Examples of some of these EPSPS include those described and/or isolated in accordance with U.S. Pat. No. 4,940,835, U.S. Pat. No. 4,971,908, U.S. Pat. No. 5,145,783, U.S. Pat. No. 5,188,642, U.S. Pat. No. 5,310,667. They can also be derived from a structurally distinct class of non-homologous EPSPS genes, such as the class II EPSPS genes isolated from Agrobacterium sp. strain CP4 (AGRTU.aroA:CP4) as described in U.S. Pat. No. 5,633,435 and 5,627,061. Alternatively, a glyphosate degrading enzyme could be used to confer glyphosate tolerance, for example using a glyphosate oxidoreductase gene as described in U.S. Pat. No. 5,312,910.

“Modulate”.

The term “modulate” refers to the regulation of translation of a gene caused by interaction of an RNA-binding protein with one or more operators in the 5′ untranslated region of the gene such that the gene product (i.e., polypeptide encoded by the gene) is expressed at a different level or has a different expression pattern (including, for example, cell, tissue, or organ specificity, temporal or developmental pattern, etc.), responsiveness to a chemical or environmental signal (including, for example, light, heat, cold, drought, salinity, etc.), or other change in expression relative to an otherwise similar control gene lacking the operator(s) in its 5′ untranslated region.

“Native”.

The term “native” refers to a naturally occurring (“wild-type”) nucleic acid or polypeptide.

“Nucleic Acid”.

The term “nucleic acid” refers to deoxyribonucleic acid (DNA) and ribonucleic acid (RNA).

“Operably Linked”.

A first nucleic-acid sequence is “operably” linked with a second nucleic acid sequence when the first nucleic acid sequence is placed in a functional relationship with the second nucleic acid sequence. For instance, a promoter is operably linked to a protein-coding sequence if the promoter affects the transcription or expression of the coding sequence. Generally, operably linked DNA sequences are contiguous and, where necessary to join two protein-coding regions, are in the same reading frame.

“Operator”, “op”.

The term “operator” (abbreviated “op”) refers to a nucleotide sequence to which an RNA-binding protein binds specifically.

“Plant”.

The term “plant” encompasses any higher plant and progeny thereof, including monocots (e.g., lily, corn, rice, wheat, barley, etc.), dicots (e.g., tomato, potato, soybean, cotton, tobacco, etc.), and includes parts of plants, including reproductive units of a plant (e.g., seeds), fruit, flowers, etc.

“Recombinant”.

A “recombinant” nucleic acid is made by an artificial combination of two otherwise separated segments of sequence, e.g., by chemical synthesis or by the manipulation of isolated segments of nucleic acids by genetic engineering techniques.

“Reproductive Unit” (of a Plant).

A “reproductive unit” of a plant is any totipotent part or tissue of the plant from which one can obtain a progeny of the plant, including, for example, seeds, cuttings, tubers, buds, bulbs, somatic embryos, cultured cells (e.g., callus or suspension cultures), etc.

“RNA-Binding Protein”.

The term “RNA-binding protein” refers to any protein that can bind specifically to a particular mRNA sequence (called an “operator”) with sufficient affinity that binding of the RNA-binding protein causes a modulation of translation of the mRNA when the operator is appropriately positioned in the mRNA, preferably in the 5′ UTR of the mRNA. Examples of RNA-binding proteins that are useful for the practice of the present invention include but are not limited to: RNA bacteriophage coat proteins, including, MS2 and Qβ phage coat proteins; RNA-binding ribosomal proteins from yeast, such as the RPL32 RNA-binding protein. The term “RNA-binding proteins” also encompasses alleles, homologs, and variants of native amino-acid sequences for such RNA-binding proteins.

“RNA-Binding Protein Nucleic Acid”.

The term “RNA-binding gene (or nucleic acid)” refers to a native nucleic acid (e.g., a cDNA or genomic sequence) or a fragment or variant form thereof that encodes a polypeptide that binds specifically to an operator sequence on an RNA with sufficient avidity to modulate translation.

“Sterility Gene”.

A “sterility gene” is a known DNA sequence that encodes a polypeptide that affects the metabolism, functioning or development of the reproductive tissue (see, e.g., EP 344,029, EP 412,911 and U.S. Pat. No. 5,633,441). A “male-sterility gene” encodes a polypeptide that affects male reproductive cells and thus can cause male sterility. A “female-sterility gene” encodes a polypeptide that affects female reproductive cells and thus can cause female sterility. Examples of polypeptides encoded by male- and/or female-sterility DNA include but are not limited to: RNases (e.g., RNase T1 and barnase); DNases (e.g., endonucleases, including but not limited to restriction endonucleases); proteases (e.g., papain); enzymes involved in phytohormone biosynthesis, that degrade a phytohormone, or that convert a phytohormone precursor into a compound that does not serve as a phytohormone precursor; glucanases, lipases, lipid peroxidases; plant cell wall inhibitors; callase; phytotoxic proteins (e.g., diphtheria toxin or botulin); proteins that render cells susceptible to a specific disease; polypeptides that cause cells to develop in an abnormal fashion (e.g., plant analogs of homeotic genes); glycoproteins such as are encoded by the S1, S2, S3, S6 and S7 alleles, e.g., of Nicotiana alata; etc.

“Translational Repression System (TRS)”.

The term “translational repression system” or “TRS” refers to the use of a translational repressor protein and its cognate operator sequence to modulate expression of a transgenic gene product in plants. TRS includes expression of RNA bacteriophage coat proteins and their operators, yeast RPL32 and its operator, among others, in plants.

Recombinant Nucleic Acid Constructs

The terms “construct” or “vector” refers to any plasmid, cosmid, virus, autonomously replicating sequence, phage, or other linear or circular single-stranded or double-stranded DNA or RNA derived from any source that includes one or more DNA sequences, such as promoters, protein-coding sequences, 3′ untranslated regions, etc., that have been linked in a functionally operative manner by recombinant DNA techniques. Recombinant vectors for plant transformation are commonly double-stranded circular or linear DNA molecules, although other vector systems are suitable for the practice of the present invention including but not limited to binary artificial chromosome (BIBAC) vectors (Hamilton et al., Gene 200:107-116 (1997)), and RNA viral vectors (Della-Cioppa et al., Ann. N.Y. Acad. Sci. 792: 57-61 (1996)), for example. Optionally, the DNA construct includes a replication system. Conventional compositions and methods for making and using recombinant nucleic acid constructs are discussed, inter alia, in Molecular Cloning: A Laboratory Manual, 2nd ed., vol. 1-3, ed. Sambrook et al., Cold Spring Harbor Laboratory Press, Cold Spring Harbor, N.Y. (1989); and Current Protocols in Molecular Biology, ed. Ausubel et al., Greene Publishing and Wiley-Interscience, New York (1992) (with periodic updates). See also, e.g., Maliga et al., Methods in Plant Molecular Biology, Cold Spring Harbor Press (1995); Birren et al., Genome Analysis: Detecting Genes, 1, Cold Spring Harbor, N.Y. (1998); Birren et al., Genome Analysis: Analyzing DNA, 2, Cold Spring Harbor, N.Y. (1998); and Clark et al., Plant Molecular Biology: A Laboratory Manual, Springer, New York (1997).

Methods for chemical synthesis of nucleic acids are discussed, for example, in Beaucage and Carruthers, Tetra. Letts. 22:1859-1862 (1981), and Matteucci et al., J. Am. Chem. Soc. 103:3185 (1981). Chemical synthesis of nucleic acids can be performed, for example, on commercial automated oligonucleotide synthesizers.

A number of vectors suitable for stable transformation of plant cells or for the establishment of transgenic plants have been described in, e.g., Pouwels et al., Cloning Vectors: A Laboratory Manual (1985, supp. 1987); Weissbach and Weissbach, Methods for Plant Molecular Biology, Academic Press (1989); and Gelvin et al., Plant Molecular Biology Manual, Kluwer Academic Publishers (1990). Typically, plant expression vectors include one or more transcription units, each of which includes a 5′ untranslated region, which includes sequences that control transcription (e.g., cis-acting promoter sequences such as enhancers, the transcription initiation start site, etc.) and translation (e.g., a ribosome binding site) of an operably linked protein-coding region (i.e., a “promoter”); a protein-coding region (or “open reading frame” or ORF); and a 3′ untranslated region that includes additional regulatory regions from the 3′ end of plant genes (Thornburg et al., Proc. Natl. Acad. Sci. USA 84:744 (1987); An et al., Plant Cell 1:115 (1989)), e.g., a 3′ terminator region to increase mRNA stability. In addition, such constructs commonly include a selectable or screenable marker and optionally an origin of replication or other sequences required for replication of the vector in a host cell.

Plant expression vectors optionally include RNA processing signals, e.g., introns, which may be positioned upstream or downstream of a polypeptide-encoding sequence in the transgene. An intron element is identified by “I-” preceeding a gene name, coding sequence name or genomic identification number. In addition, the expression vectors may also include additional regulatory sequences from the 3′-untranslated region of plant genes. These 3′ untranslated regions contain transcription termination signals, thus these regions when used in chimeric expression cassettes are designated with “T-” followed by a gene name, coding sequence name or genomic identification number. Other movable elements contained in plant expression vectors may include 5′ leader sequences, designated by “L-” and transit signal sequences designated by “TS-”, each followed by a gene name, coding sequence name or genomic identification number. The elements of a plant expression cassette are described in a 5′ to 3′ orientation of the linked elements using the element names separated by a “/”.

Promoters.

The term “promoter” or “promoter region” refers to a nucleic acid sequence, usually found upstream (5′) to a coding sequence, that controls expression of the coding sequence by controlling production of messenger RNA (mRNA) by providing the recognition site for RNA polymerase and/or other factors necessary for start of transcription at the correct site. As contemplated herein, a promoter or promoter region includes variations of promoters derived by means of ligation to various regulatory sequences, random or controlled mutagenesis, and addition or duplication of enhancer sequences. The promoter regions disclosed herein, and biologically functional equivalents thereof, are responsible for driving the transcription of coding sequences under their control when introduced into a host as part of a suitable recombinant vector, as demonstrated by its ability to produce mRNA. A “P-” preceeding a gene, coding sequence name or genomic identification number designates the element as a promoter. The genus species of the source of the promoter element is used in the promoter name, for example, Zea mays is abbreviated to Zm, Cauliflower mosaic virus is CaMV, Triticum aesativum is Ta. The 35S promoter of CaMV is therefore, P-CaMV.35S. Chimeric promoters created by fusion of promoter sequences or insertion of promoter elements to form novel non-naturally occurring sequences is designated by a combination of the names of each promoter sequence that comprise the novel sequence.

For embodiments of the invention in which the use of a constitutive promoter is desirable, any well-know constitutive plant promoter may be used. Constitutive plant promoters include, for example, the cauliflower mosaic virus (CaMV) 35S promoter, which confers constitutive, high-level expression in most plant tissues (see, e.g., Odel et al., Nature 313:810 (1985)), including monocots (see, e.g., Dekeyser et al., Plant Cell 2:591 (1990); Terada and Shimamoto, Mol. Gen. Genet. 220:389 (1990)); the nopaline synthase promoter (An et al., Plant Physiol. 88:547 (1988)), the octopine synthase promoter (Fromm et al., Plant Cell 1:977 (1989)), cauliflower mosaic virus 19S promoter, figwort mosaic virus 35S promoter, sugarcane bacilliform virus promoter, commelina yellow mottle virus promoter, rice cytosolic triosephosphate isomerase promoter, adenine phosphoribosyltransferae promoter, rice actin 1 promoter, mannopine synthase promoter, histone promoter, and a tobacco constitutive promoter as disclosed in U.S. Pat. No. 5,824,872.

For other embodiments of the invention, well-known plant gene promoters that are regulated in response to environmental, hormonal, chemical, and/or developmental signals may be used, including promoters regulated by (1) heat (Callis et al., Plant Physiol. 88:965 (1988)), (2) light (e.g., pea rbcS-3A promoter, Kuhlemeier et al., Plant Cell 1:471 (1989); maize rbcS promoter, Schaffner and Sheen, Plant Cell 3:997 (1991); or chlorophyll a/b-binding protein promoter, Simpson et al., EMBO J. 4:2723 (1985)), (3) hormones, such as abscisic acid (Marcotte et al., Plant Cell 1:969 (1989)), (4) wounding (e.g., wunl, Siebertz et al., Plant Cell 1:961 (1989)); or (5) chemicals such as methyl jasmonate, salicylic acid, etc. It may also be advantageous to employ (6) organ-specific promoters (e.g., Roshal et al., EMBO J. 6:1155 (1987); Schernthaner et al., EMBO J. 7:1249 (1988); Bustos et al., Plant Cell 1:839 (1989)).

As discussed above, for production of hybrid seeds, it may be desirable to use promoters that are preferentially expressed in a reproductive tissue. Any well-known male- or female-specific plant promoter may be used. Promoters that are preferentially expressed in a male tissue, include, but are not limited to, the following: the Xy1 promoter (Bih et al., J. Biol. Chem. 274:2884-2894 (1999)), RA8 (Jeon et al., Plant Mol. Biol. 39:35-44 (1999)), Ms45 (WO 9859061), SGB6 (U.S. Pat. No. 5837850), Tap1 (WO 9827201), Osg6B (Matsuda et al., Plant Biotechnol. (Tokyo) 14:157-161 (1997)), Sta44 (CA 2165934), MS2 (Aarts et al., Plant J. 12:615-623 (1997)), Zmgl3, TA29 (WO 9325695), SLG and SLR1 (WO 9425613), RST2 (WO 9713401), ZmC5 (WO 9942587), and A3, A6, A9 (WO 9302197, U.S. Pat. No. 5,723,754) promoters, the rice YY1 and YY2 anther-specific promoters (Hihara et al., Plant Mol. Biol. 30:1181-1193 (1996)), the corn pollen-specific promoters ZmABP1 and ZmABP2 (Lopez et al., Proc. Nat. Acad. Sci. USA 93:7415-7420 (1996)), the tapetum-specific oleosin-like gene promoters in brassica (Ross et al., Plant J. 9:625-637 (1996), the pollen-specific DEFH125 gene promoter from Antirrhinum (Zachgo et al., Plant J. 11: 1043-1050 (1997)), the pollen-specific LePro 1 promoter (Yu et al., Plant Mol. Biol. 36:699-707 (1998)), the anther-specific MROS gene promoters (Matsunaga et al., Plant J. 10:679-689 (1990)), the pollen-specific polygalaturonase gene promoter from brassica (U.S. Pat. No. 5,689,053) and maize (U.S. Pat. No. 5,412,085), the pollen-specific Lat52 and Lat59 promoters (Twell et al., Development 109:705-713 (1990)), the anther-specific 1,3-betaglucanase gene promoter (U.S. Pat. No. 5,955,653), and the Zea mays profilin multigene family anther and pollen promoters (Staiger et al., Plant J. 4:631-641 (1993)). Promoter hybrids can be made that combine the functions of pollen, anther, tapetal cell and other male tissue specific expression into a single DNA molecule, for example, a fusion of the DNA sequences of the Osg6B promoter from rice and the P-Zm.Tas9 promoter (an element isolated from a corn tassel genomic library wherein the Zm.Tas9 coding sequence has homology to Zea mays profilin coding sequences) to create P-Os.Osg6B-Zm.Tas9 where the TATA box of the 5′ Osg6B promoter sequence is modified or deleted to prevent transcription from that element. An additional promoter element can be combined with the rice-corn male promoter by the same method, for example, the wheat P-Ta. 1674-19 promoter isolated from wheat sporophyll tissue, this triple promoter fusion provides broad spectrum monocot and dicot male tissue expression. The resulting promoter, P-Os.Osg6B-Zm.Tas9-Ta. 1674-19 can be combined with the regulatory RNA binding protein coding sequences of the present invention to enable high levels of protein expression in the male tissues. These hybrid promoters are useful for providing expression at all stages of male tissue development.

Promoters that are preferentially expressed in a female tissue of the plant, include, but not limited to, the following promoters: the style and stigma specific promoters (EP 412006) and S-locus specific glycoprotein gene promoters; P26, P19, B200i4-2 (WO 9839462); DefH9 (WO 9828430); cysteine-rich extension-like protein gene promoters (Goldman et al., Plant Cell 4:1041-1051 (1992)); ovule-specific 039, 0126, 0108 and 0141 gene promoters from orchid (Nadeau et al., Plant Cell 8:213-239 (1996)), the potato pistil-specific SK2 gene promoter (Ficher et al., Plant Mol. Biol. 35:425-431 (1997)); and the rice pistil-specific RPC312 gene promoter and its monocot homolog (JP 11098986). According to one embodiment of the invention, the polypeptide-encoding DNA sequence is a gene required for female fertility, such that expression of the RNA-binding polypeptide in the female reproductive tissue reduces translation of the polypeptide-encoding DNA sequence and thereby causes female sterility.

According to certain embodiments of the invention, expression of a target gene is modulated in a tissue other than a reproductive tissue. For this purpose, one may choose from a number of promoters for genes with tissue- or cell-specific or -enhanced or induced expression. Examples of promoters that are preferentially expressed in leaves and other photosynthetically active tissues include the chloroplast glutamine synthetase GS2 promoter from pea (Edwards et al., Proc. Natl. Acad. Sci. U.S.A. 87:3459-3463 (1990)), the chloroplast fructose-1,6-biphosphatase (FBPase) promoter from wheat (Lloyd et al., Mol. Gen. Genet. 225: 209-216 (1991)), the nuclear photosynthetic ST-LS 1 promoter from potato (Stockhaus et al., EMBO J. 8: 2445-2451, (1989)), the serine/threonine kinase (PAL) promoter and the glucoamylase (CHS) promoter from Arabidopsis thaliana. Also reported to be active in photosynthetically active tissues are the ribulose-1,5-bisphosphate carboxylase (RBCS) promoter from eastern larch (Larix laricina), the promoter for the Cab gene, Cab6, from pine (Yamamoto et al., Plant Cell Physiol. 35: 773-778 (1994)), the promoter for the Cab-1 gene from wheat (Fejes et al., Plant Mol. Biol. 15: 921-932 (1990)), the promoter for the Cab-1 gene from spinach (Lubberstedt et al., Plant Physiol. 104:997-1006 (1994)), the promoter for the Cab1R gene from rice (Luan et al., Plant Cell. 4:971-981 (1992)), the pyruvate, orthophosphate dikinase (PPDK) promoter from Zea mays (Matsuoka et al., Proc. Natl. Acad. Sci. U.S.A. 90: 9586-9590 (1993)), the promoter for the tobacco Lhcb1*2 gene (Cerdan et al., Plant Mol. Biol. 33:245-255 (1997)), the Arabidopsis thaliana Suc2 sucrose-H⁺ symporter promoter (Truernit et al., Planta. 196:564-570 (1995)), and the promoter for the thylakoid membrane protein genes from spinach (PsaD, PsaF, PsaE, PC, FNR, AtpC, AtpD, Cab, RbcS). Other promoters for the chlorophyll α/β-binding proteins may also be utilized in the present invention, such as the promoters for LhcB gene and PsbP gene from white mustard (Sinapis alba) (Kretsch et al., Plant Mol. Biol. 28: 219-229 (1995)).

For the purpose of expression in sink tissues of the plant, such as the tuber of the potato plant, the fruit of tomato, or the seed of soybean, canola, cotton, Zea mays, wheat, rice, and barley, it is preferred that the promoters utilized in the present invention have relatively high expression in these specific tissues. A number of promoters for genes with tuber-specific or -enhanced expression are known, including the class I patatin promoter (Bevan et al., EMBO J. 8:1899-1906 (1986); Jefferson et al., Plant Mol. Biol. 14:995-1006 (1990)), the promoter for the potato tuber ADPGPP genes, both the large and small subunits, the sucrose synthase promoter (Salanoubat et al., Gene 60:47-56 (1987); Salanoubat et al., Gene 84:181-185 (1989)), the promoter for the major tuber proteins including the 22 kDa protein complexes and proteinase inhibitors (Hannapel, Plant Physiol. 101:703-704 (1993)), the promoter for the granule bound starch synthase gene (GBSS) (Visser et al., Plant Mol. Biol. 17:691-699 (1991)), and other class I and II patatin promoters (Koster-Topfer et al., Mol. Gen. Genet. 219:390-396 (1989); Mignery et al., Gene 62:27-44 (1988)).

Other promoters can also be used to express a protein in specific tissues, such as seeds or fruits. The promoter for β-conglycinin (Chen et al., Dev. Genet. 10:112-122 (1989)) or other seed-specific promoters such as the napin and phaseolin promoters can be used. The zeins are a group of storage proteins found in Zea mays endosperm. Genomic clones for zein genes have been isolated (Pedersen et al., Cell 29:1015-1026 (1982)), and the promoters from these clones, including the 15 kDa, 16 kDa, 19 kDa, 22 kD, 27 kDa, and gamma genes, could also be used. Other promoters known to function, for example, in Zea mays include the promoters for the following genes: waxy, Brittle, Shrunken 2, Branching enzymes I and II, starch synthases, debranching enzymes, oleosins, glutelins, and sucrose synthases. A particularly preferred promoterfor Zea mays endosperm expression is the promoter for the glutelin gene from rice, more particularly the Osgt-1 promoter (Zheng et al., Mol. Cell Biol. 13:5829-5842 (1993)). Examples of promoters suitable for expression in wheat include those promoters for the ADPglucose pyrophosphorylase (ADPGPP) subunits, the granule bound and other starch synthase, the branching and debranching enzymes, the embryogenesis-abundant proteins, the gliadins, and the glutenins. Examples of such promoters in rice include those promoters for the ADPGPP subunits, the granule bound and other starch synthase, the branching enzymes, the debranching enzymes, sucrose synthases, and the glutelins. A particularly preferred promoter is the promoter for rice glutelin, Osgt-1 gene. Examples of such promoters for barley include those for the ADPGPP subunits, the granule bound and other starch synthase, the branching enzymes, the debranching enzymes, sucrose synthases, the hordeins, the embryo globulins, and the aleurone specific proteins.

Root specific promoters may also be used. An example of such a promoter is the promoter for the acid chitinase gene (Samac et al., Plant Mol. Biol. 25:587-596 (1994)). Expression in root tissue could also be accomplished by utilizing the root specific subdomains of the CaMV35S promoter that have been identified (Lam et al., Proc. Natl. Acad. Sci. U.S.A. 86: 7890-7894 (1989)). Other root cell specific promoters include those reported by Conkling et al. (Plant Physiol. 93: 1203-1211 (1990)).

Germination and early seedling growth promoter specificity could be provided to drive expression of a transgene in a germination and early seedling growth specific or intensive process. Germination and early seedling growth promoters could be used specifically to affect a gene function that is essential for germination, but its gene expression is not limited to this time in the plant growth cycle. The preferred germination specific promoter would be most highly expressed in the appropriate tissues and cells at the appropriate developmental time to inhibit the germination enzyme or gene product only during germination or early seedling growth. Tissues and cells that comprise the germination and early seedling growth stages of plants may include the radical, hypocotyl, cotyledons, epicotyl, root tip, shoot tip, meristematic cells, seed coat, endosperm, true leaves, intemodal tissue, and nodal tissue. Germination-enhanced promoters have been isolated from genes encoding the glyoxysomal enzymes isocitrate lyase (ICL) and malate synthase (MS) from several plant species (Zhang et al, Plant Physiol. 104: 857-864 (1994); Reynolds and Smith, Plant Mol. Biol. 27: 487-497 (1995); Comai et al, Plant Physiol. 98: 53-61 (1992)). Other promoters include SIP-seedling imbibition protein (Heck, G., Ph.D. Thesis, 1992, Washington University, St. Louis, Mo.) and others such as a cysteine endopeptidase promoter (Yamauchi et al, Plant Mol. Biol. 30: 321-329 (1996)). Additionally, promoters could be isolated from other genes whose mRNAs appear to accumulate specifically during the germination process, for example class I β-1,3-glucanase B from tobacco (Vogeli-Lange et al., Plant J. 5: 273-278 (1994)); canola cDNAs CA25, CA8, AX92 (Harada et al., Mol. Gen. Genet. 212: 466-473 (1988); Dietrich et al., J. Plant Nutr. 8: 1061-1073 (1992)), lipid transfer protein (Sossountzove et al, Plant Cell 3: 923-933 (1991)); or rice serine carboxypeptidases (Washio et al., Plant Phys. 105: 1275-1280 (1994)); and repetitive proline rich cell wall protein genes (Datta et al., Plant Mol. Biol. 14: 285-286 (1990)).

Chimeric promoters that contain regulatory elements from heterologous sources can be constructed by those skilled in the art to direct the expression of the first and second DNA molecules to the desired tissue or cell in the amounts necessary to achieve the desired phenotype.

The 5′ UTR can be derived from a promoter selected to express a selected protein-coding region and can be specifically modified if desired so as to increase translation of mRNA. For a review of optimizing expression of transgenes, see Koziel et al., Plant Mol. Biol. 32:393-405 (1996). The 5′ non-translated regions can be a native sequence obtained, for example, from eukaryotic (e.g., plant) genes, from bacterial or viral genes that are expressed in plant cells (e.g., genes from Agrobacterium tumefaciens (AGRTU)), or from a chimeric or synthetic gene sequence. The operator that is bound by an RNA-binding protein is inserted into the 5′ UTR of the first DNA molecule positioned with respect to the 5′ end of the mRNA and the start site for protein translation such that binding of an RNA-binding protein to the operator inhibits or substantially reduces translation of an operably linked protein-coding sequence.

Constructs or vectors may also include with the coding region of interest a nucleic acid sequence that acts, in whole or in part, to provide a useful product expressed in plants. Herbicides in which plants have been genetically engineered for tolerance included, but not limited to the present invention and their respective genes for plant resistance include, but not limited to glyphosate (over-expressed or modified 5-enolpyruvylshikimate-3-phosphate synthase (EPSPS) or glyphosate oxidoreductase (GOX)); glufosinate (phosphinothricin acetyl transferase (PAT)); sulfonylureas and imidazolinone (insensitive acetolactone synthase (ALS) and insensitive acetohydroxyacid synthase (AHAS)); bromoxynil (Bxn gene); delapon (2,2 dichloropropionic acid) (Deh gene); cyclohexanedione (insensitive acetyl-CoA carboxylase); protoporphyrinogen oxidase inhibitors (mutant insensitive protoporphyrinogen oxidase, U.S. Pat. No. 5,939,602); isoxaslutole (RP210772) (p-hydroxyphenyl pyruvate dioxygenase, WO9924586) Other target genes may include bacterial-derived genes for insect resistance, such as the cry and vip genes of Bacillus thuringiensis, Xenorhabdus and Photorhabdus insecticidal protein genes; plant-derived genes for insect resistance such as some plant storage proteins and enzymes genes for fungal and bacterial disease resistance such as glucose oxidase, meganins; pathogenesis-related genes (PR); ribosomal inactivating proteins (RIPs), and anti-fungal proteins (U.S. Pat. No. 5,691,199).

The protein coding sequence may optionally include a known sequence that encodes a signal or transit peptide, e.g., a chloroplast transit peptide (EP 0218571), or a secretion signal.

The transcriptional unit preferably includes a 3′ UTR, operably linked to the 5′ UTR and protein-coding region, that is responsible for transcription termination and contains a polyadenylation signal. The 3′ UTR can be obtained from any gene that is expressed in plant cells. The 3′ non-translated region can be obtained from any known gene that is expressed in plant cells, e.g., the 3′ UTR from the Agrobacterium tumefaciens genes encoding nopaline synthase (nos) (T-AGRTU.nos, Fraley et al., Proc. Natl. Acad. Sci. 80:4803-4807 (1983)), or octopine synthase (ocs), pea small subunit ribulose bisphosphate carboxylase/oxygenase (Rubisco) gene, T-Ps.RbcS:E9 (Coruzzi et al., EMBO J. 3:1671-1679 (1994)), soybean 7S seed storage protein gene (Schuler et al., Nuc Acids Res. 10:8225-8244 (1982)), PI-II terminator region of potato, etc.

A transcriptional unit may also include other regulatory elements. Examples of such include the Adh intron 1 (Callis et al., Genes and Develop. 1:1183-1200 (1987)), the sucrose synthase intron (Vasil et al., Plant Physiol. 91:1575-1579 (1989)), and the TMV omega (Ω) element, L-TMV.Ω (Gallie et al., The Plant Cell 1:301-311 (1989)).

Selectable or Screenable Markers.

A vector or construct may also include any gene that encodes a selectable marker to select for, or a screenable marker to screen for, plants or plant cells that contain the exogenous genetic material. Examples of selectable markers include, but are not limited to, known genes encoding resistance to antibiotics such as hygromycin, kanamycin, bleomycin, G418, streptomycin or spectinomycin, such as the neomycin phosphotransferase (neo) gene (Potrykus et al., Mol. Gen. Genet. 199:183-188 (1985), which confers resistance to kanamycin and G418) and a dihydrofolate reductase (DHFR) gene, which confers resistance to methotrexate (Thillet et al., J. Biol. Chem. 263:12500-12508 (1988)); or known genes encoding herbicide tolerance, e.g., bar, which confers bialaphos resistance; a mutant EPSP synthase gene (Hinchee et al., Bio/Technology 6:915-922 (1988)), which confers glyphosate resistance; a nitrilase gene, which confers resistance to bromoxynil (Stalker et al., J. Biol. Chem. 263:6310-6314 (1988)); and a mutant acetolactate synthase gene (ALS), which confers imidazolinone or sulphonylurea resistance.

Examples of screenable markers include a β-glucuronidase or uidA gene (GUS), which encodes an enzyme for which various chromogenic substrates are known (Jefferson, Plant Mol. Biol, Rep. 5:387-405 (1987); Jefferson et al., EMBO J. 6:3901-3907 (1987)); an R-locus gene, which encodes a product that regulates the production of anthocyanin pigments (red color) in plant tissues (Dellaporta et al., Stadler Symposium 11:263-282 (1988)); a β-lactamase gene (Sutcliffe et al., Proc. Natl. Acad. Sci. (U.S.A.) 75:3737-3741 (1978)); a gene that encodes an enzyme for which various chromogenic substrates are known (e.g., PADAC, a chromogenic cephalosporin); a luciferase gene (Ow et al., Science 234:856-859 (1986)); a xylE gene (Zukowsky et al., Proc. Natl. Acad. Sci. (U.S.A.) 80:1101-1105 (1983)), which encodes a catechol dioxygenase that can convert chromogenic catechols; an α-amylase gene (Ikatu et al., Bio/Technol. 8:241-242 (1990)); a tyrosinase gene (Katz et al., J. Gen. Microbiol. 129:2703-2714 (1983)), which encodes an enzyme capable of oxidizing tyrosine to DOPA and dopaquinone, which in turn condenses to melanin; green flourescence protein (Elliot et al., Plant cell Rep. 18:707-714 (1999)) and an α-galactosidase.

Also included are genes that encode secretable markers such as antigens that can be identified following secretion by antibody interaction (e.g., by ELISA) or enzymes that can be detected catalytically (e.g., α-amylase, β-lactamase, phosphinothricin transferase), or proteins that are inserted or trapped in the cell wall (such as proteins that include a leader sequence such as that found in the expression unit of extension or tobacco PR-S).

Probes and Primers.

Nucleic acid probes and primers can be prepared based on a native nucleic acid sequence. A “probe” is an isolated nucleic acid to which is attached a conventional detectable label or reporter molecule, e.g., a radioactive isotope, ligand, chemiluminescent agent, or enzyme. “Primers” are isolated nucleic acids that are annealed to a complementary target DNA strand by nucleic acid hybridization to form a hybrid between the primer and the target DNA strand, then extended along the target DNA strand by a polymerase, e.g., a DNA polymerase. Primer pairs can be used for amplification of a nucleic acid sequence, e.g., by the polymerase chain reaction (PCR) or other conventional nucleic-acid amplification methods.

Probes and primers are generally 15 nucleotides or more in length, preferably 20 nucleotides or more, more preferably 25 nucleotides, and most preferably 30 nucleotides or more. Such probes and primers preferably hybridize specifically to a target nucleic acid sequence under high stringency hybridization conditions, although for identifying genes that encode homologs of a particular RNA-binding protein such probes or primers may hybridize to a native sequence of another species under moderately stringent conditions.

Methods for preparing and using probes and primers are described, for example, in Sambrook et al., 1989; Ausubel et al., 1992; and Innis et al., PCR Protocols: A Guide to Methods and Applications, Academic Press: San Diego, 1990. PCR-primer pairs can be derived from a known sequence, for example, by using computer programs intended for that purpose such as Primer (Version 0.5, 0 1991, Whitehead Institute for Biomedical Research, Cambridge, MA).

“Similarity” or “Identity”.

A first nucleic acid is “substantially similar” or “substantially identical” to a second nucleic acid if, when optimally aligned (with appropriate nucleotide insertions or deletions) with the other nucleic acid (or its complementary strand), there is at least about 75% nucleotide sequence identity, preferably at least about 80% identity, more preferably at least about 85% identity, and most preferably at least about 90% identity. Sequence similarity can be determined by comparing the nucleotide sequences of two nucleic acids using sequence analysis software such as the Sequence Analysis Software Package of the Genetics Computer Group, University of Wisconsin Biotechnology Center, Madison, Wis. One widely used and accepted computer program for performing sequence alignments is CLUSTALW v1.6 (Thompson et al. Nucl. Acids Res., 22: 4673-4680 (1994)). The number of matching bases or amino acids is divided by the total number of bases or amino acids, and multiplied by 100 to obtain a percent identity. For example, if two 580 base pair sequences had 145 matched bases, they would be 25 percent identical. If the two compared sequences are of different lengths, the number of matches is divided by the shorter of the two lengths. For example, if there are 100 matched amino acids between 200 and a 400 amino acid proteins, they are 50 percent identical with respect to the shorter sequence. If the shorter sequence is less than 150 bases or 50 amino acids in length, the number of matches are divided by 150 (for nucleic acid bases) or 50 (for amino acids), and multiplied by 100 to obtain a percent identity.

Alternatively, two nucleic acids are substantially similar if they hybridize under high stringency hybridization conditions.

With respect to nucleic acid amplification, a primer is substantially similar to a sequence to which it anneals if amplification using that primer and a second primer that anneals to a single unique sequence in the target nucleic acid results in a single amplification product.

Nucleic-Acid Hybridization; “Stringent Conditions”; “Specific”.

The nucleic-acid probes and primers of the present invention hybridize under stringent conditions to a target DNA sequence.

The term “stringent conditions” is functionally defined with regard to the hybridization of a nucleic-acid probe to a target nucleic acid (i.e., to a particular nucleic-acid sequence of interest) by the specific hybridization procedure discussed in Sambrook et al., 1989, at 9.52-9.55. See also, Sambrook et al., 1989 at 9.47-9.52, 9.56-9.58; Kanehisa, Nucl. Acids Res. 12:203-213 (1984); and Wetmur and Davidson, J. Mol. Biol. 31:349-370 (1968). Appropriate stringency conditions that promote DNA hybridization are, for example, 6 x sodium chloride/sodium citrate (SSC) at about 45° C., followed by a wash of 2 x SSC at 50° C. For example, the salt concentration in the wash step can be selected from a low stringency of about 2.0 x SSC at 50° C. to a high stringency of about 0.2 x SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed. For example, hybridization using DNA or RNA probes or primers can be performed at 65° C. in 6x SSC, 0.5% SDS, 5x Denhardt's, 100 μg/mL nonspecific DNA (e.g., sonicated salmon sperm DNA) with washing at 0.5x SSC, 0.5% SDS at 65° C., for high stringency.

Regarding the amplification of a target nucleic-acid sequence (e.g., by PCR) using a particular amplification primer pair, “stringent conditions” are conditions that permit the primer pair to hybridize only to the target nucleic-acid sequence to which a primer having the corresponding wild-type sequence (or its complement) would bind and preferably to produce a unique amplification product.

The term “specific for (a target sequence)” indicates that a probe or primer hybridizes under given hybridization conditions only to the target sequence in a sample comprising the target sequence.

Nucleic-Acid Amplification.

Nucleic-acid amplification can be accomplished by any of the various nucleic-acid amplification methods known in the art, including the polymerase chain reaction (PCR). A variety of amplification methods are known in the art and are described, inter alia, in U.S. Pat. Nos. 4,683,195 and 4,683,202 and in PCR Protocols: A Guide to Methods and Applications, ed. Innis et al., Academic Press, San Diego (1990).

Nucleotide and Amino Acid Sequence Variants of Native RNA-Binding Proteins.

Using the nucleotide and the amino-acid sequence of known RNA-binding proteins, well-known methods can be used to identify DNA molecules and polypeptides that have minor variations in their nucleotide or amino acid sequence. “Variant” DNA molecules are DNA molecules containing minor changes in a native RNA-binding protein sequence, i.e., changes in which one or more nucleotides of the native sequence is deleted, added, and/or substituted, preferably while substantially maintaining RNA-binding avidity and specificity. Variant DNA molecules can be produced, for example, by standard DNA mutagenesis techniques or by chemically synthesizing the variant DNA molecule or a portion thereof. Such variants preferably do not change the reading frame of the protein-coding region of the nucleic acid and preferably encode a protein having no change, only a minor reduction, or an increase in RNA binding.

Amino-acid substitutions are preferably substitutions of single amino-acid residues. DNA insertions are preferably of about 1 to 10 contiguous nucleotides and deletions are preferably of about 1 to 30 contiguous nucleotides. Insertions and deletions are preferably insertions or deletions from an end of the protein-coding or non-coding sequence and are preferably made in adjacent base pairs. Substitutions, deletions, insertions or any combination thereof can be combined to arrive at a final construct.

Preferably, variant nucleic acids according to the present invention are “silent” or “conservative” variants. “Silent” variants are variants of a native RNA-binding protein sequence in which there has been a substitution of one or more base pairs but no change in the amino-acid sequence of the polypeptide encoded by the sequence. “Conservative” variants are variants of a native RNA-binding protein sequence in which at least one codon in the protein-coding region of the gene has been changed, resulting in a conservative change in one or more amino acid residues of the polypeptide encoded by the nucleic-acid sequence, i.e., an amino acid substitution. A number of conservative amino acid substitutions are listed below. In addition, one or more codons encoding cysteine residues can be substituted for, resulting in a loss of a cysteine residue and affecting disulfide linkages in the polypeptide.

TABLE 1
Conservative Amino Acid Substitutions

| Original Residue | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function are made by selecting substitutions that are less conservative than those listed above, e.g., causing changes in: (a) the structure of the polypeptide backbone in the area of the substitution; (b) the charge or hydrophobicity of the polypeptide at the target site; or (c) the bulk of an amino acid side chain. Substitutions generally expected to produce the greatest changes in protein properties are those in which: (a) a hydrophilic residue, e.g., seryl or threonyl, is substituted for (or by) a hydrophobic residue, e.g., leucyl, isoleucyl, phenylalanyl, valyl or alanyl; (b) a cysteine or proline is substituted for (or by) any other residue; (c) a residue having an electropositive side chain, e.g., lysyl, arginyl, or histadyl, is substituted for (or by) an electronegative residue, e.g., glutamyl or aspartyl; or (d) a residue having a bulky side chain, e.g., phenylalanine, is substituted for (or by) one not having a side chain, e.g., glycine.

Plant-preferred Codon Usage.

In order to optimize translation in plant cells of a polypeptide that is not native to the plant, codons of the DNA sequences encoding the polypeptide may be substituted with codons that are more highly expressed in the plant, e.g., codons that are more highly represented in highly expressed polypeptides of the plant. Plant codon usage tables are well known in the art.

Modification of the nucleic acid sequence for any transgene may be necessary to improve the RNA stability or translatability of the transgene mRNA in plants, these modifications are known to those skilled in the art (Gutierrez et al., Trends Plant Sci 4:429-438 (1999)). Removal of potential polyadenylation signal sequences, intron splice sites and long or repetitive sequence regions of adenosine (A) and thymidine (T), balancing of guanosine (G) and cytidine (C) nucleotide content, and improving codon usage for plant expression are examples of modifications of the nucleic acid sequence that is well known in the art of genetic engineering of transgenes in plants (U.S. Pat. No. 5,500,365). The present invention provides for modification of the transgene nucleic acid sequence for the purpose of improved plant expression.

Polypeptides

An RNA-binding protein can be produced by the expression of a DNA sequence that encodes such a protein or by chemical synthesis (as described, for example, in Merrifield, J. Amer. Chem. Soc. 85:2149-2156 (1963)).

Polypeptide Sequence Homology.

Ordinarily, RNA-binding proteins encompassed by the present invention are at least about 70% homologous to a native RNA-binding protein, preferably at least about 80% homologous, and more preferably at least about 95% homologous. Such homology is considered to be “substantial homology.” However, RNA-binding proteins that have a lower degree of amino-acid sequence homology but that retain the ability to specifically bind an operator sequence and modulate translation are also useful in the practice of the present invention.

Polypeptide homology is typically analyzed using sequence analysis software such as the Sequence Analysis Software Package of the Genetics Computer Group, University of Wisconsin Biotechnology Center, Madison, Wis. Polypeptide sequence analysis software matches homologous sequences using measures of homology assigned to various substitutions, deletions, substitutions, and other modifications.

“Isolated,” “Purified,” or “Homogeneous” Polypeptides.

A polypeptide is “isolated” if it has been separated from the cellular components (nucleic acids, lipids, carbohydrates, and other polypeptides) that naturally accompany it. Such a polypeptide can also be referred to as “pure” or “homogeneous” or “substantially” pure or homogeneous. A polypeptide is considered “isolated” if chemically synthesized or recombinant (i.e., the product of the expression of a recombinant DNA construct, even if expressed in a homologous cell type). A monomeric polypeptide is isolated when at least 60% by weight of a sample is composed of the polypeptide, preferably 90% or more, more preferably 95% or more, and most preferably more than 99%. Protein purity or homogeneity is indicated, for example, by polyacrylamide gel electrophoresis of a protein sample, followed by visualization of a single polypeptide band upon staining the polyacrylamide gel; high pressure liquid chromatography; or other conventional methods.

Variant Forms of RNA-binding Proteins.

“Variant” polypeptides include substitutions, deletions, insertions or other modifications of a native RNA-binding protein sequence. The variants substantially retain biological activity—specifically, RNA binding—of a corresponding native RNA-binding protein and are preferably silent or conservative substitutions of one or a small number of contiguous amino acid residues.

Fusion Polypeptides.

The present invention also provides fusion polypeptides including, for example, heterologous fusion polypeptides in which an RNA-binding protein is fused to a fusion partner. Any conventional fusion partner can be used, including, for example dimers or other multimers of an RNA-binding polypeptide or two or more different RNA-binding polypeptides. Fusion polypeptides are preferably made by the expression of recombinant nucleic acids produced by standard techniques.

Plant Transformation and Regeneration

The compositions and methods of the present invention can be used to modulate gene expression in any plant that can be transformed by well-known techniques, including but not limited to corn, wheat, rice, canola, oat, barley, alfalfa, carrot, cotton, oilseed rape, sugar beet, sunflower, soybean, tomato, cucumber and squash, forest trees, ornamental annual plants, and perennial bedding plants.

A cell, tissue, organ, or organism into which has been introduced a foreign nucleic acid (or “transgene”) such as a recombinant nucleic acid vector is considered “transformed” or “transgenic.” A “transformed” cell or organism also includes progeny of a transformed cell, tissue, organ or organism and progeny produced from a breeding program employing such a transformed plant as a parent in a cross and exhibiting an altered phenotype resulting from the presence of a recombinant nucleic acid construct.

Any well-known method can be employed for plant transformation, cell and tissue culture and regeneration in the practice of the present invention with regard to a particular plant species. Conventional methods for introduction of foreign DNA into plant cells include, but are not limited to: (1) Agrobacterium-mediated transformation (see, e.g., Lichtenstein and Fuller In: Genetic Engineering, Vol 6, Rigby, ed., London, Academic Press (1987); and Lichtenstein and Draper, in: DNA Cloning, Vol II, Glover, ed., Oxford, IRI Press (1985)); (2) particle delivery (see, e.g., Gordon-Kamm et al., Plant Cell 2:603 (1990); or BioRad Technical Bulletin 1687), (3) microinjection (see, e.g., Green et al., Plant Tissue and Cell Culture, Academic Press, New York (1987)), (4) polyethylene glycol (PEG) procedures (see, e.g., Draper et al., Plant Cell Physiol. 23:451 (1982)); Zhang and Wu, Theor. Appl. Genet. 76:835 (1988)), (5) liposome-mediated DNA uptake (see, e.g., Freeman et al., Plant Cell Physiol. 25:1353 (1984)), (6) electroporation (see, e.g., Fromm et al., Nature 319:791 (1986)); and (7) vortexing method (see, e.g., Kindle, Proc. Natl. Acad. Sci. USA 87:1228 (1990)).

Any known method may be employed in order to introduce nucleic acid constructs into plant cells. General methods for delivering a gene into cells include, but are not limited to: (1) chemical methods (Graham et al., Virology 54:536-539 (1973)); (2) physical methods such as microinjection (Capecchi, Cell 22:479-488 (1980)); electroporation (Wong et al., Biochem. Biophys. Res. Commun. 107:584-587 (1982); Fromm et al., Proc. Natl. Acad. Sci. (U.S.A.) 82:5824-5828 (1985); Fromm et al., Nature 312:791-793 (1986); U.S. 5,384,253); and particle bombardment (Johnston et al., Methods Cell Biol. 43:353-365 (1994); Potrykus, Ciba Found. Symp. 154:198-208 (1990); Potrykus, Ann. Rev. Plant Physiol. Plant Mol. Biol. 42:205-225 (1991); Vasil, Plant Mol. Biol. 25:925-937 (1994)); (3) infection with Agrobacterium or other infectious agent, including viral vectors (Clapp, Clin. Perinatol. 20:155-168 (1993); Lu et al., J. Exp. Med. 178:2089-2096 (1993); Eglitis et al., Biotechniques 6:608-614 (1988)); and (4) receptor-mediated mechanisms (Curiel et al., Hum. Gen. Ther. 3:147-154 (1992), Wagner et al., Proc. Natl. Acad. Sci. USA 89:6099-6103 (1992)).

Microprojectile bombardment of plant cells has been reviewed by Yang et al., (Particle Bombardment Technology for Gene Transfer, Oxford Press, Oxford, England (1994)). An illustrative embodiment of a method for delivering DNA into Zea mays cells involves use of a biolistics α-particle delivery system, which can be used to propel particles coated with DNA through a screen, such as a stainless steel or Nytex screen, onto a filter surface covered with corn cells cultured in suspension. Gordon-Kamm et al., describes the basic procedure for coating tungsten particles with DNA (Gordon-Kamm et al., Plant Cell 2:603-618 (1990)). The screen disperses the tungsten nucleic acid particles so that they are not delivered to the recipient cells in large aggregates. A particle delivery system suitable for use with the present invention is the helium acceleration PDS-1000/He gun is available from Bio-Rad Laboratories (Bio-Rad, Hercules, Calif.) (Sanford et al., Technique 3:3-16 (1991)). Other target tissues for bombardment may be used, including immature embryos, callus tissue, etc.

Plastid transformation via microprojectile bombardment is also well-known (Svab et al. Proc. Natl. Acad. Sci. (U.S.A.) 87:8526-8530 (1990)); Svab et al., Proc. Natl. Acad. Sci. (U.S.A.) 90:913-917 (1993); Staub et al., EMBO J. 12:601-606 (1993), U.S. Pat. No. 5, 451,513 and U.S. Pat. No. 5,545,818).

The use of Agrobacterium-mediated plant integrating vectors to introduce DNA into plant cells is well known in the art. See, for example the methods described by Fraley et al., (Bio/Technology 3:629-635 (1985)) and Rogers et al., (Methods Enzymol. 153:253-277 (1987)).

Modern Agrobacterium transformation vectors are capable of replication in E. coli as well as Agrobacterium, allowing for convenient manipulations as described (Klee et al., In: Plant DNA Infectious Agents, Hohn and Schell, eds., Springer-Verlag, New York, pp. 179-203 (1985)). Vectors useful for Agrobacterium-mediated plant transformation are described, for example, in Rogers et al., Methods Enzymol. 153:253-277 (1987).

It is also to be understood that two different transgenic plants can also be mated to produce offspring that contain two independently segregating added, exogenous genes. Selfing of appropriate progeny can produce plants that are homozygous for both added, exogenous genes that encode a polypeptide of interest. Back-crossing to a parental plant and out-crossing with a non-transgenic plant are also contemplated, as is vegetative propagation. Descriptions of other breeding methods that are commonly used for different traits and crops can be found in one of several reference books (e.g. Fehr, Breeding Methods for Cultivar Development, Wilcox J. ed., American Society of Agronomy, Madison Wis. (1987)).

Transformation of plant protoplasts can be achieved using methods based on calcium phosphate precipitation, polyethylene glycol treatment, electroporation, and combinations of these treatments (see, e.g., Potrykus et al., Mol. Gen. Genet. 205:193-200 (1986); Lorz et al., Mol. Gen. Genet. 199:178 (1985); Fromm et al., Nature 319:791 (1986); Uchimiya et al., Mol. Gen. Genet. 204:204 (1986); Marcotte et al., Nature 335:454-457 (1988)). Application of these systems to different plant varieties depends upon the ability to regenerate that particular plant strain from protoplasts. Illustrative methods for the regeneration of cereals from protoplasts are described (Fujimura et al., Plant Tissue Culture Letters 2:74 (1985); Toriyama et al., Theor Appl. Genet. 205:34 (1986); Yamada et al., Plant Cell Rep. 4:85 (1986); Abdullah et al., Biotechnology 4:1087 (1986)).

Other methods of cell transformation can also be used and include but are not limited to introduction of DNA into plants by direct DNA transfer into pollen (Hess et al., Intern Rev. Cytol. 107:367 (1987); Luo et al., Plant Mol Biol. Reporter 6:165 (1988)), by direct injection of DNA into reproductive organs of a plant (Pena et al., Nature 325:274 (1987)), or by direct injection of DNA into the cells of immature embryos followed by the rehydration of desiccated embryos (Neuhaus et al., Theor. Appl. Genet. 75:30 (1987)).

The regeneration, development, and cultivation of plants from single plant protoplast transformants or from various transformed explants is well known in the art (Weissbach et al., In: Methods for Plant Molecular Biology, Academic Press, San Diego, Calif., (1988)). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

The development or regeneration of plants containing the foreign, exogenous gene is well known in the art. Preferably, the regenerated plants are self-pollinated to provide homozygous transgenic plants. Otherwise, pollen obtained from the regenerated plants is crossed to seed-grown plants of agronomically important varieties of the crop species. Conversely, pollen from plants of these important lines is used to pollinate regenerated plants. A transgenic plant of the present invention containing a desired exogenous nucleic acid is cultivated using methods well known to one skilled in the art.

There are a variety of methods for the regeneration of plants from plant tissue. The particular method of regeneration will depend on the starting plant tissue and the particular plant species to be regenerated.

Methods for transforming dicots, primarily by use of Agrobacterium tumefaciens, and obtaining transgenic plants have been published for cotton (U.S. Pat. No. 5,004,863, U.S. Pat. No. 5,159,135, U.S. 5,518,908); soybean (U.S. Pat. No. 5,569,834, U.S. Pat. No. 5,416,011, McCabe et. al., Bio/Technology 6:923 (1988), Christou et al., Plant Physiol. 87:671-674 (1988)); Brassica (U.S. 5,463,174); peanut (Cheng et al., Plant Cell Rep. 15:653-657 (1996), McKently et al., Plant Cell Rep. 14:699-703 (1995)); and pea (Grant et al., Plant Cell Rep. 15:254-258, (1995)).

Transformation of monocotyledons using electroporation, particle bombardment, and Agrobacterium have also been reported. Transformation and plant regeneration have been achieved in asparagus (Bytebier et al., Proc. Natl. Acad. Sci. (USA) 84:5354-5349 (1987)); barley (Wan et al., Plant Physiol 104:37-48 (1994)); Zea mays (Rhodes et al., Science 240:204-207 (1988), Gordon-Kamm et al., Plant Cell 2:603-618 (1990), Fromm et al., Bio/Technology 8:833-839 (1990), Koziel et al., Bio/Technology 11:194-200 (1993), Armstrong et al., Crop Science 35:550-557 (1995)); oat (Somers et al., Bio/Technology 10:1589-1594 (1992)); orchard grass (Horn et al., Plant Cell Rep. 7:469-472 (1988)); rice (Toriyama et al., Theor Appl. Genet. 205:34-(1986), Part et al., Plant Mol. Biol. 32:1135-1148, (1996), Abedinia et al., Aust. J. Plant Physiol. 24:133-141 (1997), Battraw et al., Plant Mol. Biol. 15:527-538 (1990), Christou et al., Bio/Technology 9:957-962 (1991)); rye (De 1a Pena et al., Nature 325:274-276 (1987)); sugarcane (Bower et al., Plant J. 2:409-416 (1992)); tall fescue (Wang et al., Bio/Technology 10:691-696 (1992)); and wheat (Vasil et al., Bio/Technology 10:667-674 (1992); U.S. Pat. No. 5,631,152).

Assays for gene expression based on the transient expression of cloned nucleic acid constructs have been developed by introducing the nucleic acid molecules into plant cells by polyethylene glycol treatment, electroporation, or particle bombardment (Marcotte et al., Nature 335:454-457 (1988); Marcotte et al., Plant Cell 1:523-532 (1989); McCarty et al., Cell 66:895-905 (1991); Hattori et al., Genes Dev. 6:609-618 (1992); Goffet al., EMBO J. 9:2517-2522 (1990)). Transient expression systems may be used to functionally dissect gene constructs (see generally, Maliga et al., Methods in Plant Molecular Biology, Cold Spring Harbor Press (1995)).

Site-specific or Homologous Recombination.

Operator sequences for binding of RNA-binding proteins may also be introduced into endogenous plant genes (e.g., into the 5′ UTR such that the operator is positioned in the 5′ UTR of MRNA produced by transcription of the endogenous plant genes) by site-specific or homologous recombination (see, e.g., WO 9119796 and WO 9937755) to render the endogenous plant genes susceptible to translational modulation. Agronomically useful plant phenotypes can be achieved by translational modulation of such genes as those involved in fruit ripening, flower and fruit color development, fertility, abscission, senescence, phytohormone synthesis, storage protein genes, light responsive/shade avoidance, or lignin production, for example. Targeted cell death or inhibition of cell growth and development, which is useful, for example, for producing male- or female-sterile plants, can also be achieved by modulation of translation of genes that are essential to cell viability, such as nuclear encoded mitochondrial electron transport genes, organelle protein import genes, genes for proteins involved in cell division and growth such as, microtubule protein genes (tubulin), actin, and ubiquitin.

Glyphosate as a Chemical Hybridizing Agent (CHA) for Hybrid Seed Production

One aspect of the present invention relates to improved methods for producing male- or female-sterile plants wherein a herbicide such as glyphosate is used as a gametocide or chemical hybridizing agent (CHA). The effective use of glyphosate in this capacity, and the use of gametocides in general, has been limited by the lack of sufficient selectivity for male gametes so as to prevent unnecessary damage to female gametes and vegetative tissue. According to one embodiment of the invention, an RNA-binding protein is expressed in a plant under the transcriptional control of a promoter that is expressed preferentially in pollen or another male reproductive tissue. The plant also comprises a herbicide-tolerance gene under the control of a constitutive promoter that includes an operator sequence located such that the mRNA resulting from transcription of the herbicide-tolerance gene includes the operator in the 5′ UTR. Binding of the RNA-binding protein to the operator sequence in the male-reproductive tissue reduces or eliminates translation of the mRNA, resulting in herbicide sensitivity in the male-reproductive tissue. Upon application of herbicide, the plant remains vegetatively tolerant and female fertile but is rendered male-sterile. Similarly, expression of the RNA-binding protein under the control of a promoter that is expressed preferentially in a female reproductive tissue reduces or eliminates translation of the mRNA in the female tissue, rendering the plant female-sterile but male-fertile upon application of the herbicide. Such a system can be used to confer vegetative tolerance and reproductive (male or female) sensitivity to an herbicide such as glyphosate through the use of target DNA sequences that encode a glyphosate-resistant EPSP synthase or a glyphosate degrading enzyme (e.g., glyphosate oxidoreductase [GOX]), for example.

The female parent of a hybrid seed production method is by necessity male sterile. In order to render male reproductive tissue susceptible to glyphosate-induced toxicity transgenic plant lines are selected from a population of lines containing a constitutive type promoter driving the expression of a RNA-binding protein operator sequence (op) and a glyphosate tolerance enzyme encoded by the first DNA molecule; a male tissue specific promoter driving expression of a RNA-binding protein encoded by the second DNA molecule. The plant lines are selected that show glyphosate sensitivity of male reproductive structures, such that when varying amounts of glyphosate are applied to these plant lines the plants are vegetatively and female tolerant but male sensitive to the phytotoxic effects of glyphosate. These plant lines become the female parent in a hybrid seed production method.

The male parent of the hybrid seed production method can be a nontransgenic pollen producer or a transgenic plant line produced by selecting vegetative and fully male fertile glyphosate tolerant lines and female glyphosate sensitive lines from a population of lines containing a constitutive type promoter driving the expression of the glyphosate tolerance enzyme, and containing a second DNA molecule with a female tissue-specific promoter that directs expression exclusively or primarily in female reproductive tissue of a DNA sequence that causes the production of a RNA sequence encoding a RNA-binding protein. In this way, glyphosate tolerance is selectively attenuated in a female-specific manner by the tissue-specific inhibition of expression of the first DNA molecule encoding the glyphosate tolerance enzyme.

The skilled individual will recognize that numerous approaches can be used to arrive at a transgenic plant containing the first and second DNA molecules of the invention for the production of male sterile/female fertile and male fertile/female sterile plants. The DNA molecules can be introduced into a plant in any appropriate manner and/or order, e.g., simultaneously, separately, sequentially etc. For example, where the first and second DNA molecules are introduced separately to produce independent lines, the two plant lines can be crossed using traditional breeding methods and progeny from the cross assayed for the presence of the transgenes. The progeny containing both transgenes are allowed to self and progeny from this self can be assayed for the presence of both transgenes. Those populations that are homozygous for both genes are tested for response to glyphosate application on male sterility or female sterility, and vegetative glyphosate tolerance. Lines exhibiting effective vegetative tolerance to glyphosate and demonstrating the desired level of male or female sterility are further propagated.

Alternatively, the expression cassettes comprising the first DNA molecule and the second DNA molecule may be contained on the same plasmid, and transformed into the plant cells as a single piece of DNA and regenerated into plants. A subset of the seeds or other propagative structures from the regenerated plants are planted and the plants treated with glyphosate. Those exhibiting the desired level of vegetative glyphosate tolerance and the desired level of male or female sterility are saved. Sibling seeds are planted and propagated for seed increase. Methods such as molecular marker analysis involving the use of microsatellite markers (SSRs), AFLP markers, RFLP markers, RAPD markers, phenotypic markers, single nucleotide polymorphisms (SNPs), isozyme markers, or microarray transcription profiles that are genetically linked to or correlated with alleles of a transgenes of the present invention can be utilized (Walton, Seed World 22-29 (1993); Burow et al., Molecular Dissection of Complex Traits, 13-29,Paterson, CRC Press, New York (1988)). Analysis can be used to identify the transgenic lines and maintain the line identity for seed increase without the need for application of glyphosate. Plants regenerated from transformed plant cells comprising the first and second DNA molecules of the invention are vegetatively and either male sensitive to glyphosate, but female tolerant; or are female sensitive and male tolerant to glyphosate. In the absence of glyphosate spray, the plants are normal and fully fertile. This allows for very straightforward line maintenance via selfing. When glyphosate is sprayed on the plants of the present invention, complete male or female sterility can result.

The disclosed method for generating male or female sterile plants is readily adapted to the production of hybrid seed, including hybrid seed with restored fertility. Thus, relating to an additional embodiment of the present invention, there is provided a method of producing hybrid seed that comprises first regenerating a plant from a transformed plant cell that contain the first DNA molecules described above, selecting individual lines from the population of transformed plants, which when exposed to glyphosate produce male sterile, female fertile plants. During production of hybrid seed, the seed parent plants are sprayed with glyphosate and rendered male sterile, female fertile and pollinated by the pollen parent that are male fertile, female sterile. Thus, F1 hybrid seed produced in this way will not contain significant amounts of the male pollinator parent.

The techniques described herein are applicable to essentially all crop species, including monocotyledonous plants such as rice, wheat, oats, barley, corn and the like, as well as dicotyledonous plants such as alfalfa, canola, carrot, cotton, oil seed rape, sugar beet, sunflower, soybean, tomato, cucumber and melons, squash, and others. This method can be applied to ornamental plants of many varieties to engineer the plants for hybrid seed production. Plants genetically engineered to contain the glyphosate tolerance genes are fully fertile in the absence of glyphosate. This trait allows for the stable increase of the parental seed prior to the production of the hybrid cross. This hybridization system is applicable to all plants for which transformation is possible. Crops of particular interest include the monocot crops: corn (Zea mays), wheat (Triticum aestivum) and rice (Oryzae sativa); and the dicot crops: cotton (Gossypium sp.) and soybean (Glycine max).

In addition, crops that the vegetative parts of the plant are the primary agricultural product such as sugar beet, sugarcane, potatoes, sweet potatoes; leafy vegetables such as lettuce, cabbage, spinach, and tea; vegetable root crops such as carrots, radish, turnips, garlic and onions selected to be vegetatively resistant to the toxic effects of glyphosate, but male sensitive when produced according to the present invention and treated with glyphosate, would be prevented from forming pollen. Hence, glyphosate could be used to control weeds in these crops and the plants, once sprayed, would become male sterile limiting the potential out-crossing of this gene or other genes introduced into the crop plants by classical breeding or biotechnological methods, such as genes engineered or breed for insect resistance, disease resistance, drought tolerance, freeze tolerance or other traits that may contribute to increased weediness of related wild species of the engineered crop.

Interplanting.

The efficiency of hybrid seed production can be improved by interplanting male parent plants that are male-fertile and female-sterile with female parent plants that male-sterile, female-fertile as a result of the application of an herbicide, preferably glyphosate. By interplanting the male and female parent plants according to a random distribution, the male and female parent plants are in closer proximity than would be possible if they were segregated in rows, resulting in efficient pollination. The ratio of male to female parent plants required for efficient pollination can be easily determined for each crop for which the method is applied by a simple field experiment. The numbers of male fertile plants needed in this method is less than the numbers of male fertile plants needed in current hybrid seed production systems. In some systems it is expected that 10%, 5% or less than 5% of the plants need to consist of male pollenators for effective pollination to occur. Male pollenators can be further enhanced by selecting for plants that shead copious amounts of pollen over a longer time period and are taller than the female parent.

Use of the Translational Repression System in Connection with “Sterility Genes”

Reduction of translation of a herbicide-tolerance gene such as a glyphosate-tolerance gene (e.g., CP4) does not affect the metabolism or development of a plant cell directly, as the plant cell retains a normal phenotype until herbicide application. However, the translational repression system of the present invention is also useful in connection with “sterility genes” that cause male or female sterility by directly affecting the metabolism or development of reproductive tissues.

One approach is to express a “sterility gene” in a male or female reproductive tissue of a parental line and suppress its expression in hybrid progeny produced by a sexual cross such that the hybrid progeny are fertile. For example, a male-sterile female parent can be produced by transforming a plant with a nucleic acid construct that comprises a dominant sterility gene such as barnase or trehalose phosphate phosphatase (TPP) (U.S. Pat. No. 5,689,041; WO 99/23233) operably linked to a promoter that causes transcription of the sterility gene in a male reproductive tissue (e.g., pollen or tapetal cells) and includes a 5′ UTR that includes an operator to which an RNA-binding protein can bind and suppress translation of the sterility gene. The sterility gene may be expressed only in the male reproductive tissue, in which case a promoter specific for the male reproductive tissue is used. Alternatively, if expression of the sterility significantly affects only the male-reproductive tissue, the sterility gene may be expressed in the male reproductive tissue and in other tissues as well, e.g., constitutively. Male-fertile progeny can be produced by crossing the male-sterile female parent with a male-fertile male parent that is transformed with a nucleic acid construct comprising a gene encoding an RNA-binding protein that is expressed under the control of a promoter that causes the RNA-binding protein to be expressed in the male reproductive tissue (either a constitutive promoter or a non-constitutive promoter that is expressed in the male reproductive tissue). In the progeny of such a cross, binding of the RNA-binding protein to the operator reduces translation of the sterility gene.

A similar approach may be used to cause female sterility, in which case the female-sterile plant is transformed with a nucleic acid construct that comprises a sterility gene operably linked to a promoter that causes transcription of the sterility gene in a female reproductive tissue and includes a 5′ UTR that includes an operator to which an RNA-binding protein can bind and suppress translation of the sterility gene. Female fertility is likewise restored by crossing the female-sterile plant with a plant that expresses the RNA-binding protein in the female reproductive tissue.

Alternatively, it is possible to produce using a plant that is sterile as a result of a mutation in (or absence of) a gene that encodes a polypeptide that is required for normal plant metabolism, functioning or development (a “fertility DNA”). The plant could be rendered conditionally fertile by transforming the plant with a DNA construct that comprises: (1) a gene complementing the mutation and having in its 5′ UTR an operator sequence to which an RNA-binding protein binds; and (2) a gene encoding the RNA-binding protein that is operably linked to a promoter that is chemically inducible. The transformed plant is normally fertile as a result of the expression of the fertility gene. However, application of a compound that induces expression of the RNA-binding protein causes a repression of translation of the fertility DNA and thus causes sterility.

Other Agricultural Uses of the Translational Repression System

The translation repression system (TRS) of the present invention is also useful for modulation of transgene expression in pollen in any crop or ornamental plant for the purpose of reducing insecticidal protein production in pollen which in turn reduces the potential to adversely affect nontarget insects. Tissue-specific expression of the translation repression system can also be used to limit the expression of numerous target transgenes, e.g., insecticidal protein genes, plant hormone biosynthesis genes, plant hormone perception genes, pharmaceutical protein genes, disease resistance genes, and other genes of economic importance, in a tissue-specific manner to reduce environmental exposure of the gene product, or to affect tissue-specific growth patterns due to the sensitivity or insensitivity to plant hormones. Plant hormones such as ethylene, gibberellins, cytokinins, and auxins can be used to alter plant and plant organ growth and development. However, the effect of systemic expression may render the plant not commercially viable. The translation repression system allows for tissue specific regulation of the effect. For example, ethylene is a plant hormone involved in ripening and senescence, decreasing ethylene production or ethylene perception is a method used in plant biotechnology to delay fruit ripening (U.S. Pat. No. 5,702,933, and U.S. Pat. No. 5,955,652) and fruit abscission, however, root growth, and plant development can be adversely affected by constitutive expression of the plant hormone related gene product. In this example, root specific promoters and the translation repression system expressed in cotton, soybean, and other row crops will limit the physiological effects of the plant hormone gene product to those target tissues of interest.

In addition, glyphosate sensitivity can be used as a harvest aid. In cotton production, late season regrowth of cotton after defoliation can result in greening of the lint during the harvest. Senescence active promoters, late developmental stage promoters, and chemical inducible promoters in combination with the translation repression system of the present invention will prevent regrowth of cotton plants after application of glyphosate.

Use of Translational Repression System Together With Means for Modulating Transcription

Molecular gene expression switches in plants that couple the translation repression system described in the present invention with transcription control can add an additional level of gene control. Transcription control has been described in transgenic plants that use a chemical inducible switch that consists of bacterial operator sequences (such as tetO from E.coli transposon Tn10, Gatz et al., Mol. Gen. Genet. 227:229-237 (1991); Roder et al., Mol. Gen. Genet. 243:32-38 (1994)) around the promoter TATA box. Other Tet regulatory systems (U.S. Pat. No. 5,650,298) can also be used in combination with the translational repression system. These systems in combination with the repressor operator sequences of the present invention (viral protein binding sites from bacteriophages such as MS2 and Qβ, and yeast Rp132 operator) in the 5′ UTR of the targeted genes give a high level of gene expression control. The switches are controlled by the expression of a fusion protein of the bacterial TetR (operator sequence) with the viral MS2 coat protein or RegA (T4 bacteriophage) or Rpl32 protein gene and their cognate sequences. Upon the addition of chemical inducers (tetracycline and tetracycline analogs), the transcription repression conferred by the fusion protein will be released, yet the translation repression maintains. This is important for uses where complete repression of the gene expression is essential in the absence of an inducer. Control of highly cytotoxic protein expression (e.g., such as bacterial barnase from Bacillus, fungal glucose oxidase from Aspergillus, isopentenyltransferase from Agrobacterium and cholesterol oxidase from Streptomyces) is necessary when targeting specific cell or tissue types in plants. The combination of a transcriptional repressor and translational repressor gives tight control of protein expression. The compound switches will be potent in controlling the expression of cyto-inhibitory genes. Target genes include genes involved in the biosynthesis and response of plant hormones/growth substances such as gibberellin, abscisic acid, auxin, cytokinin, ethylene, polyamines, jasmonates, salicylic acid and brassinolides; also the genes involved in lipid (such as thiolase and PEPCK), protein (asparagine synthetase and glutamine synthetase) and carbohydrate (invertase) mobilization; biotin biosynthesis (BioA, B, C, D and F) and sequestration (such streptavidin or avidin).

The invention will be better understood by reference to the following Examples, which are intended to merely illustrate the best mode now known for practicing the invention. The scope of the invention is not to be considered limited thereto.

### EXAMPLES

### Example 1

### Cloning of the MS2 Coat Protein Gene

Bacteriophage MS2 is obtained from American Type Culture Collection (ATCC) as accession no. 15597-B 1) along with the E. coli host strain (no. 15597). The host strain is cultured in a 250 mL flask containing 60 mL Luria broth (LB) media at 37° C. and agitated at 100 rpm in a New Brunswick table top shaker Model G24 to an OD₆₅₀ of about 0.5 and inoculated with 1 mL MS2 phage stock at 10⁸ pfu/mL. Cells are cultured for an additional three hours at 37° C. under the same conditions. The incubation mixture is centrifuged at 15,000 x g for 10 minutes in a Sorvall SS34 rotor, and the supernatant passed through a 0.2 μM filter to remove remaining bacterial cells. The MS2 titer is determined to be in excess of 10¹⁶ plaque forming units (pfu)/mL. MS2 bacteriophage is precipitated from 10 mL of the above preparation by addition of 2.5 mL of 30% polyethylene glycol (PEG) 8000; 1.6 M NaCl. The precipitate is collected by centrifugation at 30,000 x g for 15 min in a Sorvall SS34 rotor, dissolved in 0.5 mL TE (10 mM Tris-HCl, pH 7.5, 1 mM ethylene diamine tetraacetic acid (EDTA)), and extracted three times with acid-phenol (pH 4.5):chloroform:isoamyl alcohol (125:25:1), and once with chloroform:isoamyl alcohol (25:1). RNA is precipitated with three volumes of ice cold 100% ethanol, the precipitate is collected by centrifugation at 30,000 x g for 15 min. in a Sorvall SS34 rotor and the precipitate is washed with 70% ethanol. Eighteen μg of RNA is obtained; the RNA migrated at the correct size on a 1.2% formaldehyde gel and is >90% intact. The nucleotide sequence for the MS2 coat protein gene is obtained from Genbank accession number: V00642 (nucleotide positions 1335-1727). Oligonucleotide primers (SEQ ID NO: 1; SEQ ID NO:2) are designed to clone the coat protein gene with flanking endonuclease restriction sites, NcoI/MS2 coat protein gene/PstI.

The MS2 coat protein gene is obtained from the MS2 RNA genome template by use of these primers and the GeneAmp Thermostable rtth Reverse Transcriptase RNA PCR kit (Perkin Elmer, Wellesley, Mass., part number: N808-0069) following the vendor's protocol and using buffers supplied by the vendor. For reverse transcription, 150 ng MS2 RNA, 2 μL 10x reverse transcription buffer, 1 mM MnCl₂, 200 μM each dGTP, dATP, dTTP, and dCTP, 5 units rTth DNA polymerase, 0.75 μM reverse primer (SEQ ID NO:2), and sufficient ultrapure water to make a total reaction volume of 20 μL are mixed. This reaction is incubated for 15 min. at 70° C., and then placed on ice. To the reverse transcription reaction is added 8 μL ‘10’ chelating buffer, 8 μL 25 mM MgCl₂, 0.15 μM (final concentration) of forward primer (SEQ ID NO: 1) and sufficient ultrapure water to make a total reaction volume of 100 μL. PCR conditions are as follows: step 1: 95° C., 120 sec.; step 2: 95° C., 60 sec.; step 3: 60° C., 60 sec.; step 4: repeat steps 2-3 for a total of 35 times; step 5: 60° C. for 7 min.

Forward Primer MS2 CP (SEQ ID NO: 1):

5′-ATGAATTCCATGGCTTCTAACTTTACTCAGTTCGTTCTCGTCG

Reverse Primer MS2 CP (SEQ ID NO:2):

5′-TACTGCAGCTATTAGTAGATGCCGGAGTTTGCTGCGATTGCTGAGGG

Mutagenesis of the MS2 Coat Protein Gene for Enhanced Plant Expression.

A plant expression enhanced version of the MS2 CP gene (SEQ ID NO:3) is constructed that has the codon usage of monocot plant (corn codon usage table, GCG, Madison Wis.). The GC content of the MS2 CP gene is changed from 49% to 67% after the modification for enhanced plant expression (U.S. Pat. No. 5,500,365), whereas the native amino acid sequence of the MS2 CP (Genbank #j02467, Genbank CAA23989) is retained. The synthetic MS2 CP gene is generated from oligonucleotides by splicing by overlap extension polymerase chain reaction (SOE PCR) (Horton et al., Gene 77:61-68 (1989); Yon et al., Nucl. Acids Res., 17:4894 (1989)). Oligonucleotides are purchased from Gibco BRL (Gaithersburg, Md.) having the following sequences:

synms2pr1 (SEQ ID NO:4):
5′-ATGGCCTCCAACTTCACCCAGTTCGTGCTCGTGGACAACGGCGGCAC
CGGCGACGTGACCGTCGCCCCAAGCAACTTCGCCAACGGGRTCGCCGAGTGG
A-3′
synms2pr2 (SEQ ID NO:5):
5′-TGATGGTGTACTTGCGGTTCTGCGCGGAGGACTGGCGCACGCTGCAG
GTCACCTTGTAGGCCTGGGAGCGCGAGTTGGAGCTGATCCACTCGGCGAYCC
C-3′
synms2pr3 (SEQ ID NO:6):
5′-CGCAAGTACACCATCAAGGTCGAGGTGCCGAAGGTGGCCACCCAGAC
CGTCGGCGGCGTGGAGCTGCCAGTGGCCGCCTGGCGCTCGTACCTCAACATG
G-3′
synms2pr4 (SEQ ID NO:7):
5′-GGTTGCCGTCCTTCAGGAGGCCTTGCATGGCCTTCACGATCAGCTCGC
AGTCGGAGTTCGTGGCGAAGATTGGGATGGTCAGCTCCATGTTGAGGTACGA-3′
synms2pr5 (SEQ ID NO:8):
5′-CTGAAGGACGGCAACCCGATCCCCTCCGCCATCGCCGCCAACTCCGG
CATCTACTAA-3′
synms2pr6 (SEQ ID NO:9):
5′-CCGGGAATTCCTTAGTAGATGCCGGAGTTGGC-3′
synms2pr7 (SEQ ID NO:10):
5′-GGCCGGATCCACCATGGCCTCCAACTTCACCCAG-3′
synms2pr8.1 (SEQ ID NO:11):
5′-GGCCGGATCCATGGCCAGCATGACCGGCGGCCAGCAAATGGGCACCA
TGGCCTCCAACTTCACCCAGTTCGTGCTC-3′

Restriction sites (underlined in the primer SEQ ID NOs:9, 10, 11) are incorporated into the primers to facilitate cloning. Oligonucleotides synms2pr1 and synms2pr2 each contained a degenerate nucleotide (R=A or G, Y=C or T) that results in the synthesis of a modified version of the MS2 CP gene. This mutant MS2 CP is shown to have higher affinity to its ligand in vitro (Lim et al., Nucl. Acids Res. 22:3748-3752, (1994)). The enhanced plant expression gene is constructed by using SOE PCR with the Expand™ High Fidelity PCR system (Roche Boehriger Mannheim, Nutley, N.J. Cat.# 1732641). The PCR parameters are: step 1: 94° C., 2 min; step 2: 94° C., 20 sec; step 3: 55° C., 30 sec; step 4: 72° C., 60 sec; step 5: go to step 2, 3 cycles; step 6: 94° C. 2 min; step 7; 94° C., 20 sec; step 8: 60° C., 30 sec; step 9: 72° C., 60 sec; step 10: go to step 7, 25 cycles; step 11: 72° C., 5 min, and step 12: hold at 4° C.

Construction of MS2 RNA-Binding Protein Gene Expression Plasmids.

The expected 450 base pair (bp) fragment containing the MS2 CP gene is obtained by PCR as described above, digested with NcoI and PstI, and cloned into pSPUTK (Stratagene, La Jolla, Calif.). The sequence is confirmed to be identical to the Genbank V00642 MS2 CP gene sequence. This vector is used for in vitro testing of the system. For expression in dicot plant cells, the NcoI-PstI (blunt, T4 DNA polymerase) coat protein-containing fragment from the above construct is subcloned into the NcoI-EcoRI (EcoR1 5′ overhang filled by reaction with Klenow fragment and dNTPs) site flanking the E. coli uidA gene (P-CaMV.35S/L-TMV.Ω/Ec.uidA/T-AGRTU.nos), replacing the uidA (GUS) gene of E. coli. This placed the MS2 CP gene under the control of the CaMV duplicated enhancer 35S promoter (P-CaMV.35S), and put the gene behind the omega (Ω) 5′-translational leader of tobacco mosaic virus (L-TMV.Ω), terminating with the nos 3′ terminator region from Agrobacterium tumefaciens; (T-AGRTU.nos).

The DNA sequence encoding the T7-epitope (MASMTGGQQMG) (T7-tag, pET vectors, Novagen, Inc., Madison, Wis.) is added by PCR to the coat protein gene to make use of the commercially available anti-T7-tag antibodies (Novagen, Inc., Madison, Wis.) to determine the level of coat protein expression. High fidelity PCR using Pwo polymerase (Boehringer Mannheim, Nutley, N.J.) is used to insert this modification on the N-terminal end of the coat protein using the primers SEQ ID NO: 12 and SEQ ID NO: 13.

Forward Primer T7 tag (SEQ ID NO:12):

5′-AAAAAGATATCCATGGCTTCCATGACTGGTGGACAGCAAATGGGAAT GGCTTCTAACTTTACTCAGTTC

Reverse Primer T7 tag (SEQ ID NO: 13):

5′-TACTGCAGCTATTAGTAGATGCCGGAGTTTGCTGCGATTGCTGAGGG

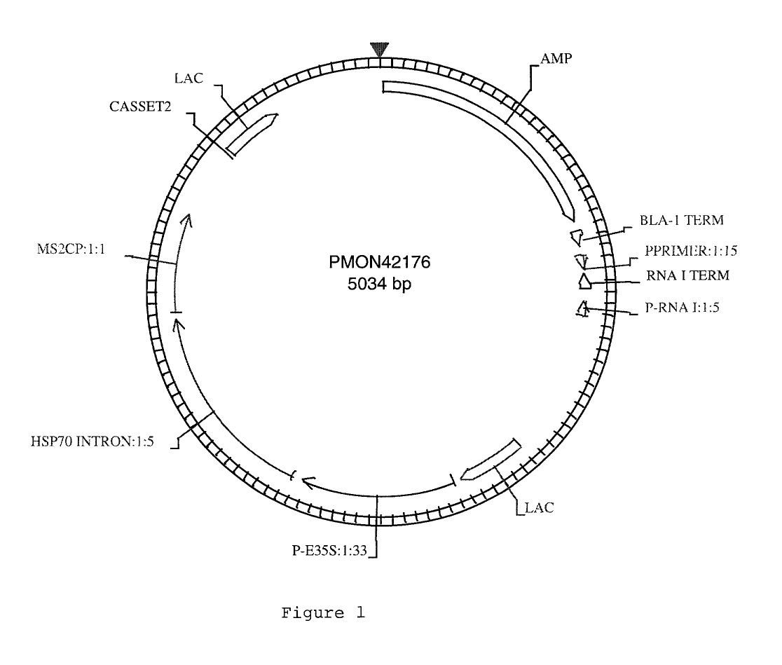

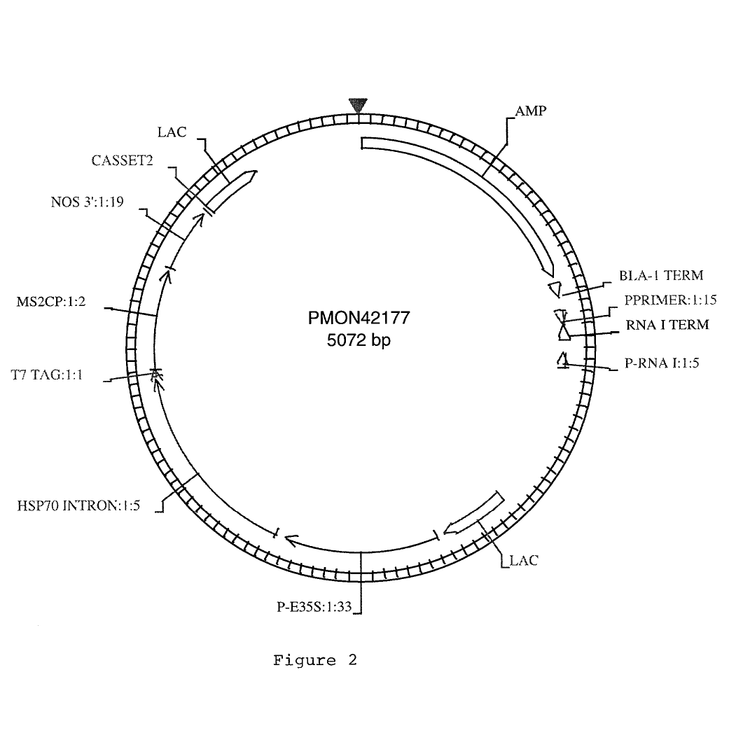

Two constructs are made to enhance MS2 coat protein gene expression in corn and wheat protoplasts and to test MS2 coat protein-mediated translational repression in monocot plants. For enhanced expression in monocot cells, the Zea mays Hsp70 intron (I-Zm.Hsp70) replaced the tobacco mosaic virus 5′-translational Ω leader, resulting in pMON42176 (FIG. 1) (P-CaMV.35S/I-Zm.Hsp70/MS2.CP/T-AGRTU.nos) (no T7 tag) and pMON42177 (FIG. 2) (P-CaMV.35S/I-Zm.Hsp7O/T7tag-MS2.CP/T-AGRTU.nos) (T7 tag), respectively.

MS2 Coat Protein Gene Fusion Multimer.

The MS2 CP binds to the translational operator as a dimer to repress translation (Witherell et al., Prog. Nucleic Acid Res. Mol. Biol., 40:185-220, (1991)). Genetic fusion of the two subunits of the MS2 CP gene in frame resulted in fusion proteins that are substantially more stable than wild-type MS2 CP as indicated by increased resistance to urea denaturation (Peabody, Arch. Biochem. Biophys. 347:85-92 (1997)). The enhanced plant expression MS2 CP gene sequence is spliced to produce a fusion gene by using SOE PCR (Horton et al., Gene 77:61-68 (1989); Yon et al., Nucl. Acids Res., 17:4894 (1989)). To construct the MS2 dimer fusion gene, the 5′ gene half is fused with the 3′ gene half at the DNA sequence of the codons encoding the amino terminal amino acid (Y130) and the fourth amino acid (N4), respectively. The translational fusion resulted in the deletion of amino acids 1-3 of the MS2 CP of the 3′ gene and the addition of an alanine residue at the fusion junction (shown in bold letters in SEQ ID NO: 14 below). The fusion junction is shown below:

MS2 CP fusion junction (SEQ ID NO: 14)
AAC TCC GGC ATC TAC GCC AAC TTC ACC CAG TTC
N S G I Y A N F T Q F

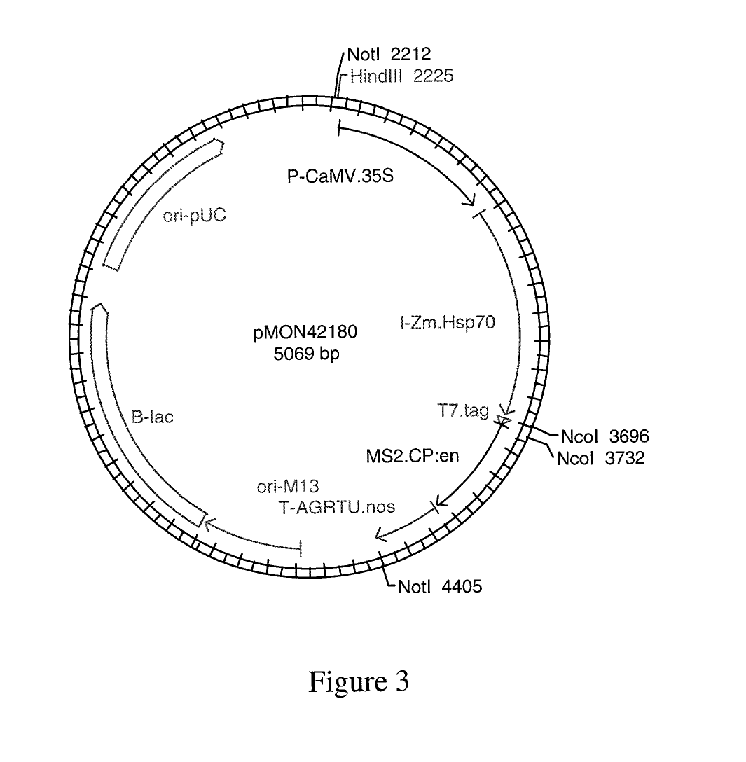

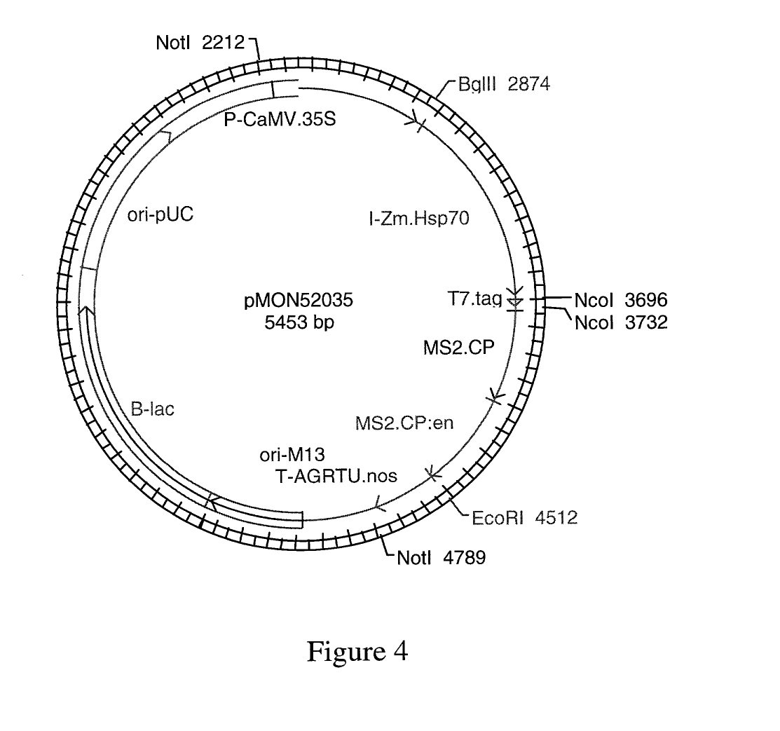

The fused gene fragment is cloned into pMON42180 (P-CaMV.35S/I-Zm.Hsp70/T7tag-MS2.CP:en/T-AGRTU.nos) (FIG. 3) and the duplicated MS2 CP gene is constructed, resulting in pMON52035 (P-CaMV.35S/I-Zm.Hsp70/T7tag-MS2.Cp-MS2.CP:en/T-AGRTU.nos) (FIG. 4).

Synthesis of the MS2 Translational Operator.

The MS2 translational operator sequence as described by Stripecke et al. (Mol. Cell. Biol. 14:5898-5909 (1994)) is made by hybridizing two oligonucleotides, SEQ ID NO: 15 and SEQ ID NO: 16, and filling in with T4 DNA polymerase (Sambrook et al., Molecular Cloning: A Laboratory Manual, 2^{nd} ed. Cold Spring Harbor Laboratory Press, Cold Spring Harbor, N.Y., (1989)). HindIII sites (underlined in SEQ ID NO: 15 and SEQ ID NO: 16) are placed at each end of the operator to facilitate cloning into the 5′-UTRs of reporter constructs. The primers are annealed and filled by the use of Klenow fragment and dNTPs, then digested with HindIII.

Forward Primer MS2 op (SEQ ID NO:15):
5′-ATAAGCTTTAAATTATCTACTTAAGGACCATCAGGCCTTAAGAACAC
Reverse Primer MS2 op (SEQ ID NO: 16):
5′-TAAAGCTTGATATCATATGTTCTCGAGTTTTGTGTTCTTAAGGCCTG
ATGGTCC

The MS2 translational operator contained in the Hind m DNA fragment is ligated into the HindIII site in pSPUTK (5′ UTR site), the correct orientation being determined by sequencing (ABI Prism™ 377, Perkin Elmer, Foster, Calif.).

To test translational repression in plant cells, the plant expression cassette (P-CaMV.35S/L-TMV.Ω/Ec.uidA/T-AGRTU.nos), is modified by mutagenesis to insert a SpeI-HindIII fragment containing the operator sequence near the 5′-terminus of the Tobacco mosaic virus Ω leader sequence. The Clonetech “Transformer” mutagenesis system is used with the mutagenesis primer SEQ ID NO: 17 (Clonetech, Palo Alto, Calif.)

Mutagenesis Primer MS2op (SEQ ID NO:17):
5′-CATTTGGAGAGGACGTACTAGTATATAAGCTTTTTACAACAATTACC
AAC

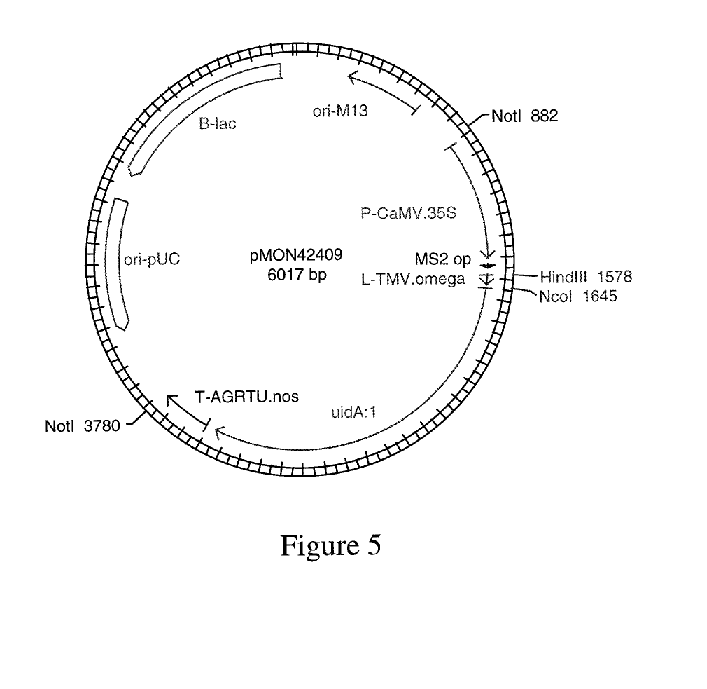

The MS2 translational operator (MS2op) contained on a HindIII fragment (pSPUTK-MS2op) is ligated into the new HindIII site in the mutagenized cassette (P-CaMV.35S/L-TMV.Ω/Ec.uidA/T-AGRTU.nos). The 5′-3′ orientation of the MS2op is determined by DNA sequencing and the correct orientation is 5′-CTTAAGGACCATCAGGCCTTAAG-3′ (SEQ ID NO:28). One construct, named pMON42409 (P-CaMV.35S/MS2op/L-TMV.Ω/Ec.uidA/T-AGRTU.nos) (FIG. 5), is used for testing translational repression in plant protoplasts.

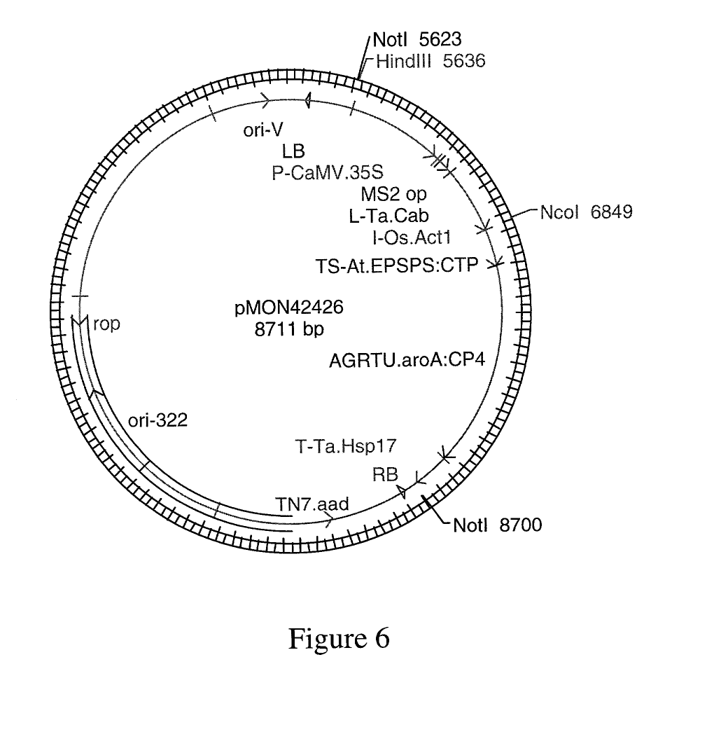

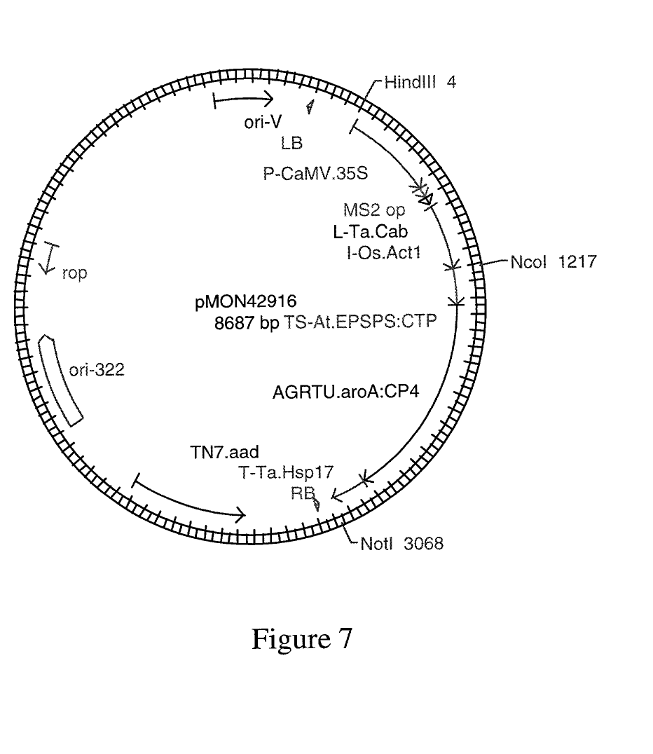

Two plasmids are constructed to contain the MS2 operator, these are pMON42426 (P-CaMV:35S/L-Ta.Cab/MS2op/I-Os.Act l/TS-At.EPSPS :CTP/AGRTU.aroA:CP4/T-Ta.Hspl7) (FIG. 6) and pMON42916 (P-CaMV:35S/L-Ta.Cab/MS2op/I-Os.Actl/TS-At.EPSPS:CTP/AGRTU.aroA: CP4/T-Ta.Hspl7) (FIG. 7). These plasmids contain the wheat chloroplast AB binding protein gene leader (L-Ta.Cab) (U.S. patent application Ser. No. 09/376940), the intron from the rice actin 1 gene (I-Os.Actl) (U.S. Pat. No. 5,641,876), the chloroplast transit signal peptide from the EPSPS gene of Arabidopsis thaliana (TS-At.EPSPS:CTP) (Klee et al., Mol. Gen. Genet. 210:437-442 (1987)), the glyphosate resistant EPSPS enzyme gene from Agrobacterium tumefaciens strain CP4 (AGRTU.aroA:CP4) (U.S. Pat. No. 5,633,435), and the 3′ nontranslated terminator region from the heat shock 17 gene of wheat (T-Ta.Hspl7) (U.S. patent application Ser. No. 09/376940. They also differ by the presence of two NotI sites in pMON42426 and only one NotI site in pMON42916, useful for further cloning steps.

### Example 2

### MS2 Coat Protein Mediated Translation Repression System in vitro

The MS2 coat protein mediated translational repression as demonstrated by inhibition of the GUS reporter gene activity in vitro. Capped mRNAs of the reporter gene and the MS2 coat protein gene are transcribed using the “mMessage mMachine” system (Ambion, Austin, Tex.). The MRNA (1 μg) is then translated in 50 μL wheat germ in vitro translation system (Ambion, Austin, Tex.). Initially, the lysates are programmed with either coat protein, MRNA, or water. Translation is allowed to progress for 10 min. at 30° C, after which the reporter mRNA is added and translation allowed to continue for an additional 30 min. GUS activity is determined for the in vitro translation carried out in the presence or absence of the coat protein. A fluorometry assay for GUS activity is performed as described (Jefferson, Plant Mol. Bio. Rep. 5:387-405 (1987)). GUS activity is measured with the fluorometric substrate 4-methylumbelliferryl-β-glucuronide (4-MUG) on a Fmax fluorimeter (Molecular Devices Corp., Sunnyvale, Calif.). Table 2 shows the results of a typical assay. The in vitro results indicate that the system components work to reduce GUS expression by a translational repression system. The addition of a T7-tag is not detrimental to the function of the MS2 coat protein as a translational repressor. Other peptide sequences may also be added to create fusion proteins with the MS2 CP to impart multiple functions to the chimeric protein.

TABLE 2
GUS activity of reporter gene in vitro

| mRNA's Added | % Inhibition of GUS |
|---|---|
| GUS | 0 |
| GUS + coat protein | 93 |
| GUS + T7-tagged coat protein | 99 |

Expression of the MS2 Translation Repression System in Corn, Wheat, and Tobacco Leaf Protoplasts.

Corn protoplasts are prepared from etiolated leaves of antiseptically grown seedlings. Six to eight grams of leaf tissue is harvested and put in the enzyme mix (1% cellulase, 0.1% macerozyme, 0.6 M mannitol, 10 mM MES (pH =5.7), 1 mM CaCl₂, 1 mM MgCl₂, and 0. 1% BSA) in 100×25 mm petri plates. The leaf digestion is done for 135 minutes, swirling on a shaker at 50 rpm at 26° C. under light (10-25 mico-einsteins) in a growth chamber (Percival Scientific Inc., Boone, Iowa). Protoplasts are screened through a 190 μm sterile tissue mesh into a petri plate. The protoplasts are spun down in a table top centrifuge at 800 rpm (˜200 g) for 8 minutes. The supernatant is removed carefully by pipetting. The protoplasts are resuspended in 10 mL per tube of the 0.6 M mannitol rinse. They are spun down a second time using the same conditions and re-suspended in 10 mL of the electroporation buffer (0.6 M mannitol, 4 mM MES, 1.0 mM β-mercaptoethanol, 25 mM KCL, pH =5.7) and put on ice. The number of protoplasts is determined by making counts on a hemacytometer and a final density of 4.5×10⁶ per mL is obtained with the proper volume of electroporation buffer. For electroporation, 0.75 mL of protoplasts is added to each cuvette followed by the addition of plasmid DNA in a volume of 50 μL or less. Samples are electroporated at 125 FFarads and 260 volts using a Bio-Rad Gene Pulser Electroporater (Bio-Rad CA) and incubated in Murishige-Skoog medium (MS) +0.6 M mannitol in the light at 26° C. for 18-22 hours before analysis. Protoplasts are harvested by spinning down at 800-1000 rpm in a table top centrifuge and lysed by resuspending in 500 μL extraction buffer (IX phosphate buffered saline [PBS] (Roche Boehringer Mannheim #1666789, Nutley, N.J.) 0.2% BSA, 0.5% Tween 20, pH 7.4) and vortexed for five seconds. Samples are spun at 3,000 rpm for 5 min in a table top centrifuge and the supernatant is saved to a fresh tube and put on ice ready for analysis.

The reporter plasmid pMON42426 (FIG. 6) and the MS2 coat protein expression plasmid pMON42180 (P-CaMV.35S/I-Zm.Hsp70/T7tag-MS2.CP:en/T-AGRTU.nos) (FIG. 3) are co-electroporated at two different DNA molar ratios into corn protoplasts to demonstrate the MS2 CP mediated translational repression of aroA:CP4 EPSPS reporter gene expression. The total amount of DNA in each sample is equalized by using as a carrier an appropriate amount of an empty vector plasmid DNA pMON999 (P-CaMV.35S/T-AGRTU.nos). With the aroA:CP4 EPSPS gene as the reporter, the GUS activity encoded by a separate plasmid is used as an internal control to normalize the expression. Similarly, the aroA:CP4 EPSPS gene expression is used for normalization when the Ec.uidA (GUS) gene is used as the reporter.

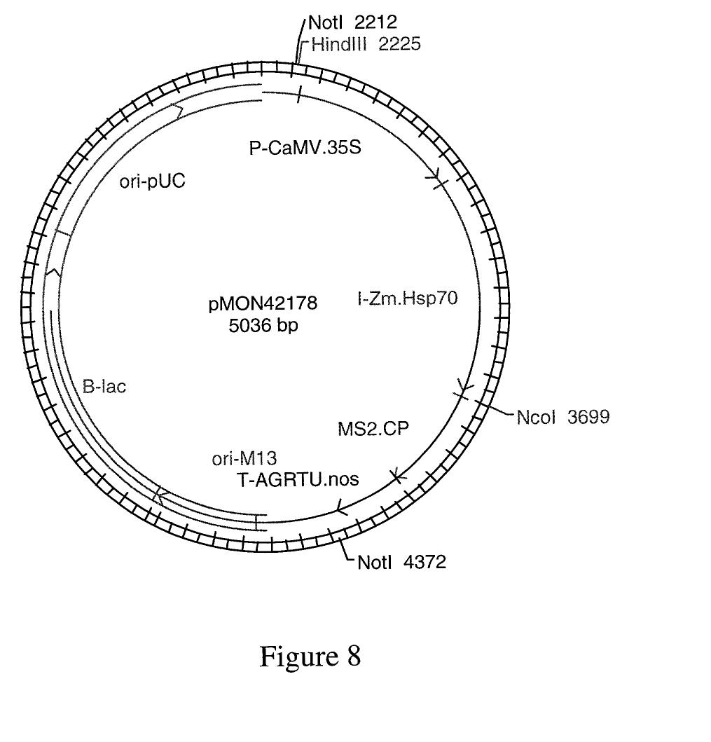

The enhanced plant expression sequence (MS2.CP:en, SEQ ID NO:3) of the MS2 CP gene is compared with its native sequence for efficiency in translational repression in plant cells. Plasmid pMON42409 (FIG. 5) contained the plant expression cassette of the enhanced CaMV 35S promoter, the Ec.uidA reporter gene with the MS2 operator incorporated in the 5′UTR and the nos 3′ nontranslated region. pMON42176 (FIG. 1) (P-CaMV.35S/I-Zm.Hsp70/MS2.CP/T-AGRTU.nos) expresses the native MS2 CP gene (Genbank #j02467, nucleotide position 1335-1727). pMON42177 (FIG. 2) (P-CaMV.35S/I-Zm.Hsp7O/T7tag-MS2.CP/T-AGRTU.nos) encodes for a native MS2 CP with the T7 epitope tag fused at its amino terminus. pMON42178 (P-CaMV.35S/1-Zm.Hsp70/MS2.CP:en/T-AGRTU.nos) (FIG. 8) and pMON42180 (FIG. 3) both contain the plant expression enhanced MS2 CP gene (MS2.CP:en), whereas pMON42180 additionally encodes for the T7tag-MS2 CP:en fusion gene. The plant expression-enhanced MS2 CP:en gene increases the efficiency of translational repression by 74-80% compared to the respective native gene construct (pMON42178pMON42176 and pMON42180/pMON42177) expressed as decrease in GUS activity (Table 3.). This result also showed that the enhanced MS2 CP:en gene is more effective in plant cells than the native MS2 CP gene sequence and the efficacy of the MS2 CP mediated translational repression is independent of the reporter gene.

TABLE 3
Enhanced plant expression of repressor protein increases efficiency of
translational repression

| Treatment | pMON DNA^{a} | GUS Activity^{b} | STDER | % Expression |
|---|---|---|---|---|
| 1 | 42409 | 19.489 | 0.40 | 100.0 |
| 2 | 42409 + 42176 | 1.973 | 0.15 | 10.1 |
| 3 | 42409 + 42177 | 1.984 | 0.85 | 10.1 |
| 4 | 42409 + 42178 | 1.135 | 0.22 | 5.8 |
| 5 | 42409 + 42180 | 1.098 | 0.40 | 5.8 |

^{a}The molar ratio of the plasmid DNAs is 1:1
^{b}GUS activity is measured as nmol 4-MU/μg protein minute. Mean of four independent electroporations.

Table 4 summarizes the result of a typical assay. This result shows that the expression of the aroA:CP4 EPSPS reporter gene is reduced to 14% of the control (or about 7-fold repression) in corn protoplasts when the MS2 coat protein gene is co-expressed. The MS2 coat protein-mediated translational repression in plant cells is dosage-dependent: a 4:1 DNA molar ratio of the coat protein gene to the reporter reduced the reporter gene expression to 0.4% of the control (or 270-fold repression).

TABLE 4
MS2 coat protein represses CP4 EPSPS expression in corn protoplasts

| | | CP4 | | |
|---|---|---|---|---|
| Treatment | Plasmid DNA | Reading^{b} | STDER | % Expression |
| 1 | 42426 | 0.27 | 0.05 | 100.0 |
| 2 | 42426 + 42180(1:1)^{a} | 0.039 | 0.01 | 14.4 |
| 3 | 42426 + 42180(1:4) | 0.001 | 0.01 | 0.4 |

^{a}Molar ratio of the target to the MS2 coat protein expression plasmid DNA
^{b}Mean of four independent electroporations, analysis by ELISA

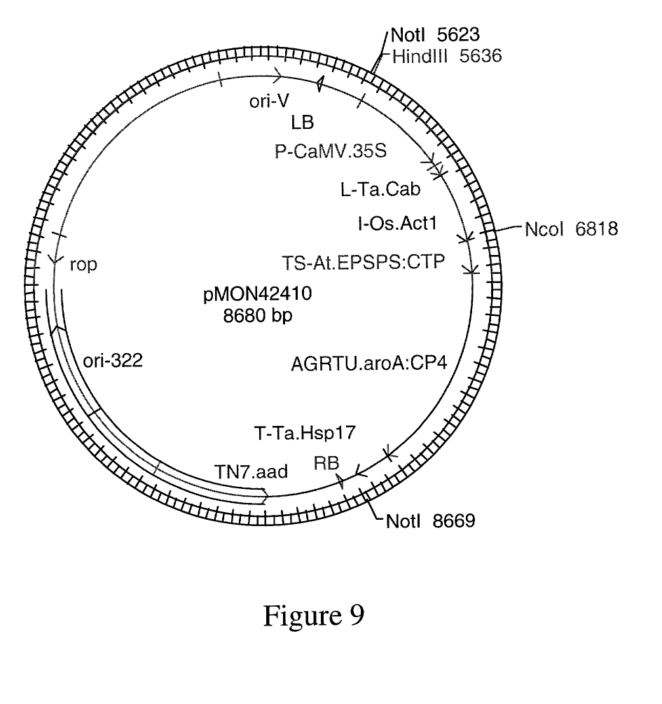

When pMON42180 is co-electroporated with pMON42410 (P-CaMV.35S/L-Ta.Cab/I-Os.Act1/TS-At.EPSPS :CTP/AGRTU.aroA:CP4/T-Ta.Hsp 17) (FIG. 9), which does not contain the translation operator in the 5′UTR of the CP4 EPSPS gene, the expression is not significantly changed (Table 5, treatment 3). Incorporation of the operator in the 5′UTR did not significantly change expression in the absence of the MS2 coat protein (Table 5, treatment 2). Taken together, these results demonstrate that repression of the CP4 EPSPS expression observed in Table 4 is caused by the specific interaction of the operator and the MS2 coat protein.

TABLE 5
Operator dependent repression of expression via MS2 coat protein

| | | CP4 | | |
|---|---|---|---|---|
| Treatment | pMON DNA | Reading^{b} | STD ER | % expression |
| 1 | 42410 | 0.399 | 0.01 | 100.0 |
| 2 | 42426 | 0.493 | 0.03 | 123.5 |
| 3 | 42410 + 42180 (1:1)^{a} | 0.474 | 0.01 | 118.8 |

^{a}Molar ratio of plasmid DNA
^{b}Mean of three independent electroporations, analysis by ELISA

Wheat protoplast preparation and electroporation are performed as described (Zhou et al., Plant Cell Reports 12:612-616 (1993)). Electroporation of protoplasts from Bobwhite wheat is conducted with pMON42409 and pMON42180. Eight grams of wheat cell suspension is resuspended with 40 mL enzyme solution and incubated at 26° C. for 2 hours on a rotator at 40 rpm. The centrifugation of the solution is conducted at 200 x g for 8 min. The protoplasts are washed twice with centrifugation between each wash. They are resuspended in 10 mL wash solution and stored on ice. The number of protoplasts is determined and the volume adjusted to make the concentration 4×10⁶ protoplasts/mL. Add 0.75 mL of protoplasts to each electroporation cuvette, then up to 50 μg plasmid DNA in 50 μL solution is added to the protoplasts. The electroporation conditions used a Bio-Rad Gene Pulser are 960 μFarads and 160 volts. The samples remained on ice for 10 min then are pipetted into MS 1 WSM media and incubated in the dark for 18-22 hours at 24° C. Cells are pelleted by centrifugation at 200-250 x g for 8 min. The pellets are frozen on dry ice.

The translational repression mediated by MS2 coat protein/operator is also demonstrated in wheat protoplasts. Table 6 shows that the MS2 coat protein/operator translation repression system works in wheat cells at a similar efficiency to that in corn cells.

TABLE 6
MS2 coat protein represses translation in wheat protoplasts

| Treatment | pMON DNA^{a} | GUS Activity^{b} | STDER | % Expression |
|---|---|---|---|---|
| 1 | 42409 | 4.343 | 1.39 | 100.0 |
| 2 | 42409 + 42180 | 0.537 | 0.15 | 12.4 |

^{a}The molar ratio of the target and MS2 plasmid DNA is 1:1
^{b}Mean of four independent electroporations, assay by ELISA

The results shown in Tables 4 and 6 demonstrate that in wheat and corn protoplasts, it is possible to inhibit glyphosate tolerance gene expression from pMON42426 and pMON42409, respectively using MS2 CP to the genetic elements expressed from the plant expression vector pMON42180. The plant expression vectors constitutively transcribed a RNA molecule containing a 5′ leader sequence, an intron, an RNA-binding site, an exon sequence comprising an RNA sequence encoding a CTP, a glyphosate tolerance gene and a 3′ untranslated region.

Translational Repression in Tobacco Protoplasts.

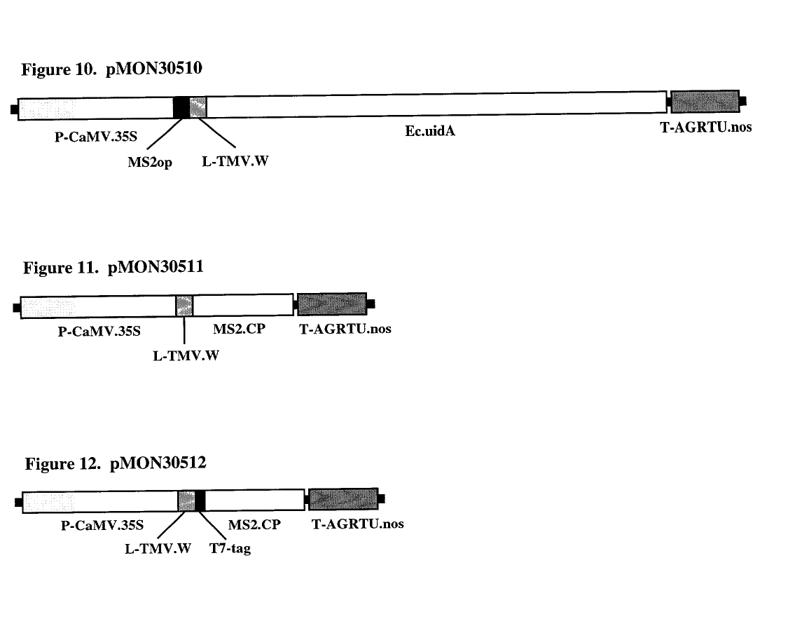

Tobacco protoplast preparation and electroporation are performed as described (Gallois et al., Methods Mol. Biol., 55:89-107 (1995)). The tobacco protoplast transient expression system is used to demonstrate that MS2 coat protein-mediated translational repression also functions in dicotyledonous plant cells. Tobacco protoplasts are electroporated with constructs containing Ec.uidA (GUS) named pMON30510 (FIG. 10) (P-CaMV.35S/MS2op/L-TMV.Ω/Ec.uidA/T-AGRTU.nos) in the presence or absence of constructs containing either the native MS2 coat protein pMON30511 (FIG. 11) (P-CaMV.35S/L-TMV.Ω/MS2.CP/T-AGRTU.nos) or the MS2 coat protein containing an N-terminal fusion with the T7 epitope pMON30512 (FIG. 12) (P-CaMV.35S/L-TMV.Ω/T7-tag MS2.CP/T-AGRTU.nos). All cells are co-electroporated with the same concentration of a plasmid containing the P-CaMV:35S/LUC/T-AGRTU.nos plant expression cassette expressing the luciferase gene (LUC) as an electroporation control. The transformed cells are allowed to grow and then analyzed for GUS and luciferase activities; GUS activity is reported as relative to luciferase expression. Table 7 shows the result of a typical assay demonstrating that the translational repression system is active in a dicot plant cell.

TABLE 7
MS2 coat protein represses translation in tobacco protoplasts

| Constructs Added | % Inhibition of GUS |
|---|---|
| pMON30510 | 0 |
| pMON30510 + pMON30511 | 76 |
| PMON30510 + pMON30512 | 78 |

CP4 EPSPS ELISA and GUS and LUC Activity Assays.

The reporter gene activities in corn, wheat and tobacco protoplast extracts from the demonstrations of the operator/repressor system are analyzed by the methods described below. The aroA:CP4 EPSPS expression is quantitated by ELISA. A crude protoplast extract containing lmg total protein is added to goat anti-CP4 EPSPS IgG coated wells of a 96-well microtiter plate for immunoreaction. Antibodies specific to CP4 EPSPS are produced in goats by immunization of three goats with recombinant SDS-PAGE purified CP4 EPSPS from recombinant E. coli. Goats are initially immunized by multiple intermuscular injections with approximately 0.4 mg of CP4 EPSPS in 4 mL of Freunds complete adjuvant. Animals are immunized on monthly intervals with injections of approximately 0.4 mg of CP4 EPSPS in 4 mL of Freunds incomplete adjuvant.

For CP4 EPSPS ELISA assay, a 96-well Nunc-Immuno MaxiSorb plate is coated with 100 μL of purified monoclonal anti-CP4 EPSPS IgG antibody (1 ng/μL) in coating buffer (15 mM Na₂CO₃, 35 mM NaHCO₃, pH 9.6). The plate is sealed and incubated at 37° C. for one hour. The plate is then rinsed three times with washing buffer (1X PBS, 0.05% Tween 20, pH7.4). Fifty μL of the supernatant (either straight or diluted with extraction buffer 1:5 or 1: 10) is added to a well, followed by addition of 50 μL of anti-CP4 EPSPS antibody-horseradish peroxidase conjugate diluted at 1:10,000 with StabilZyme (SurModics, Inc., #SZ02-1000, Eden Prarie, Minn.). The loaded plate is incubated at 37° C. for one hour then rinsed three times with washing buffer. To develop the plate, 100 μL of substrate (a 1:1 mixture of H₂O₂ and TMB (3,3′-5,5′-tetra methyl benzidine), Kirgeggard and Perry, #50-76-03, Gaithersburg, Md.) is added to each well and the plate is incubated at room temperature for 3-5 min. One hundred μL of stop solution (3M H₃PO₄) is added to terminate the reaction. The plate is read on a Spectra Max 340 (Molecular Devices, Sunnyvail, Calif. 94089) at 450 nm. Quantitation of sample CP4 EPSPS concentration is accomplished by extrapolation of the logistics curve fit of the CP4 EPSPS standard curve present on each plate. Purified CP4 EPSPS protein is produced in recombinant E. coli expressing pMON17101 (P-recA/G10L/CP4 EPSPS/T7 terminator) or another suitable expression vector for production of proteins in E. coli (Sambrook et al., Molecular Cloning: A laboratory manual, 2^{nd} ed. Cold Spring Harbor Laboratory Press, Cold Spring Harbor, N.Y., (1989)). The E. coli-produced CP4 EPSPS is used as a standard as well as as a positive control, and extracts made from protoplasts electroporated with empty vector plasmid are used as the negative control.

A fluorometry assay for GUS activity is performed as described (Jefferson, Plant Mol. Bio. Rep. 5:387-405 (1987)). GUS activity is measured with the fluorometric substrate 4-methylumbelliferryl-β-glucuronide (4-MUG) on a Fmax fluorimeter (Molecular Devices Corp., Sunnyvale, Calif.). Luciferase analysis is performed as described in U.S. Pat. No. 5,424,412.

### Example 3

### Cloning of the Qβ Coat Protein Gene

An E. coli strain harboring the Qβ RNA phage is obtained from ATCC (accession #23631-B1). The lyophilized bacterium culture is reconstituted as described in the ATCC manual and 1 μL of the reconstitute is used in a reverse transcriptase-polymerase chain reaction (RT-PCR) to amplify the Qβ CP gene. The following primers, which are used in the RT-PCR, are designed based on a published Qβ CP gene sequence (Genbank accession # m99039): JHQBU (SEQ ID NO:18):

JHQBU (SEQ ID NO:18):
5′-CGG CGG ATC CAC CAT GGC AAA ATT AGA GAC TGT
TAC-3′
JHQBL2 (SEQ ID NO:19):
5′-GGC CGA ATT CTC AAT ACG CTG GGT TCA GCT G-′3

Restriction sites (underlined in SEQ ID NO:18, 19) are incorporated at the 5′ end of both primers to facilitate cloning into expression vectors. The RT-PCR reaction is carried out using the Titan One Tube RT-PCR System (Roche Boehringer Mannheim, Cat#1888382, Nutley, N.J.) following the manufacturer's instructions.

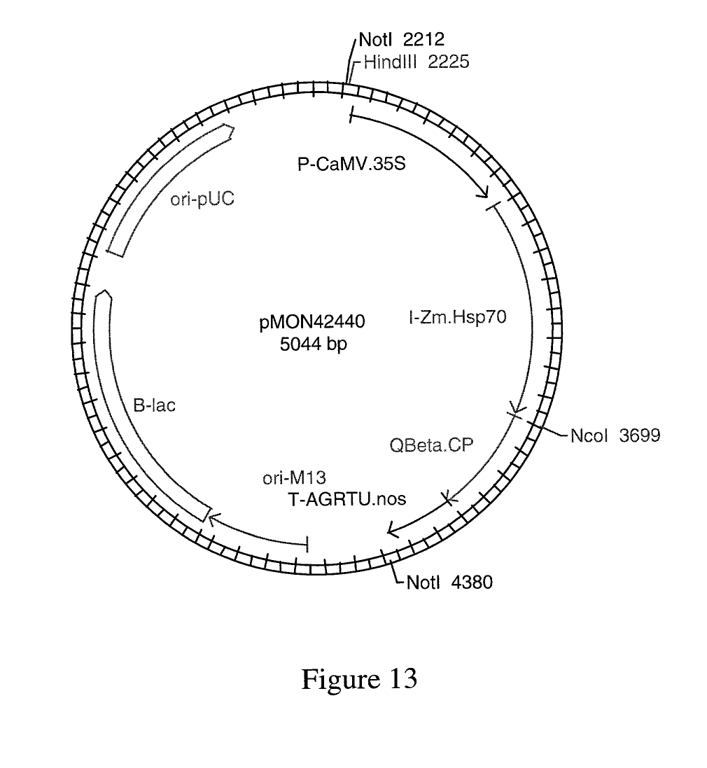

The RT-PCR-amplified Qβ coat protein gene fragment is digested with BamHI and EcoRI and ligated (Rapid DNA Ligation Kit cat# 1635378, Roche Boehringer Mannheim, Nutley, N.J.) into pMON42180, resulting in pMON42440 (FIG. 13) (P-CaMV.35S/I-Zm.Hsp70/Qβ.CP/T-AGRTU.nos). The entire region of the amplified gene is sequenced and confirmed to be identical to the published Qβ coat protein sequence (Genbank #m99039, nucleotides 46-447).

Enhanced Plant Expression Qβ Coat Protein Gene Sequence.

A plant expression enhanced version of the Qβ CP gene (SEQ ID NO:20) is constructed that has a codon usage typical of Zea mays plant genes. The codon-optimized coding sequence is obtained by reverse translation using the corn codon usage table according to the GCG protocol (SEQLAB GCG, Madison, Wis.), whereas the native amino acid sequence of the Qβ CP (Genbank #m99039) is retained. The Zea mays codon usage table of Nakamura (http://www.dna.affrc.go.jp/˜nakamura/) is an additional source of codon usage information useful for the design of genes for expression in monocot plants. The GC content of the Qβ CP gene is changed from 50.2% to 68.7%. The enhanced plant expression Qβ CP gene is generated from eight oligonucleotides by SOE PCR (Horton et al., Gene 77:61-68 (1989); Yon et al., Nucl. Acids Res., 17:4894 (1989)). Oligonucleotides are purchased from Gibco BRL (Gaithersburg, Md.). The sequence of the Qβ gene modified for enhanced expression in monocot plants is shown in SEQ ID NO:20.

Restriction sites are incorporated into the primers to facilitate cloning. The enhanced plant expression gene is constructed by using SOE PCR with the Expand™ High Fidelity PCR system (Roche Boehriger Mannheim, Nutley, N.J. Cat.# 1732641). The PCR parameters are: step 1: 94° C., 2 min; step 2: 94° C., 20 sec; step 3: 55° C., 30 sec; step 4: 72° C., 60 sec; step 5: go to step 2, 3 cycles; step 6: 94° C., 2 min; step 7; 94° C., 20 sec; step 8: 60° C., 30 sec; step 9: 72° C., 60 sec; step 10: go to step 7, 25 cycles; step 11: 72° C., 5 min, and step 12: hold at 4° C.

Reporter Plasmid for the Qβ Coat Protein.

The Qβ operator is inserted via PCR into the 5′-UTR of the CP4 EPSPS reporter gene in pMON42410 (FIG. 9). The primers used for the PCR are:

QBopL (SEQ ID NO:21):
5′-GCG CTC TAG ATG TTC TCG AGT TTT GTG TTG GAT CAG TCT TAG ACT
TGA TCT TCC TAA TTG C-3′
35S-1 5′ (SEQ ID NO:22):
5′-GTT AAC AAG CTT CTG CAG GTC CGA TGT GAG AC-3′

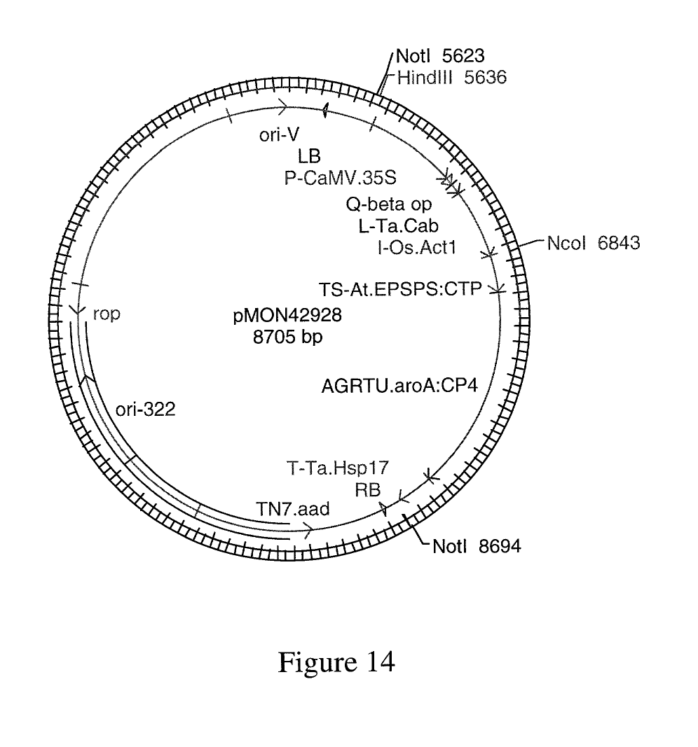

The underlined sequence in primer QBopl is complementary to the Qβ operator coding sequence. PCR is carried out by using pMON42409 as the template. The amplified fragment containing the CaMV duplicated 35S promoter and the Qβ translational operator in the correct orientation is cloned into pMON42410, resulting in pMON42928 (FIG. 14). The operator in pMON42928 is confirmed to be correct by PCR DNA sequencing method (ABI Prism™ 377, Rerkin Elmer, Foster, Calif.).

Qβ Coat Protein Represses Translation of Transgene in Corn Protoplasts.

pMON42928 is electroporated into corn protoplast either alone or together with pMON42440 (P-CAMV.35S/I-Zm.Hsp70/Qβ.CP/T-AGRTU.nos) (FIG. 13) as described in Example 2. Table 8 summarizes the results of a typical assay and shows that the Qβ coat protein also functioned to repress the expression of the CP4 EPSPS reporter gene when the operator is incorporated into its 5′-UTR.

TABLE 8
Qβ coat protein represses CP4 EPSPS expression in corn protoplasts

| | | CP4 | | |
|---|---|---|---|---|
| Treatment | pMON DNA | Reading^{b} | STDER | % Expression |
| 1 | 42928 | 0.79 | 0.05 | 100.0 |
| 2 | 42928 + 42440 (1:1)^{a} | 0.18 | 0.02 | 22.3 |
| 3 | 42928 + 42440 (1:2) | 0.12 | 0.01 | 15.2 |

^{a}The DNA molar ratio of the target plasmid to coat protein plasmid.
^{b}Mean of three independent electroporations.

Results summarized in Table 9 show that the Qβ coat protein did not repress the expression of the CP4 EPSPS reporter gene containing the MS2 operator in its 5′-UTR. This result corroborates a previous report (Lim et al., J Biol Chem 269:9006-9010 (1994)) that coat protein-mediated repression of gene expression results from the specific interaction of the coat protein and its cognate operator.

TABLE 9
Ligand specific repression of gene expression by the Qβ coat protein

| | | CP4 | | |
|---|---|---|---|---|
| Treatment | pMON DNA | Reading^{b} | STDER | % Expression |
| 1 | 42426 | 0.214 | 0.001 | 100.0 |
| 2 | 42426 + 42440 (1:1)^{a} | 0.316 | 0.03 | 147.7 |
| 3 | 42426 + 42440 (1:4) | 0.278 | 0.039 | 129.9 |

^{a}The DNA molar ratio of the target plasmid to coat protein plasmid.
^{b}Mean of three independent electroporations.

### Example 4

### Reporter Plasmids Containing Double MS2 Operators

The tandem double MS2 operator construct containing both operators in the 5′-UTR is made in two steps. PCR is initially used to insert the MS2 operator sequence into pMON42410. The primers used are:

MS2opSmaILI (SEQ ID NO:23):
5′GCGCCCCGGGATGTTCTCGAGTTTTGTGTTCTTAAGGCCTGATGGTCC
TTAAGCCCTTGGATCTTATGGTGTGTTG-3′
35S-1 5′ (SEQ ID NO:22):
5′GTTAACAAGCTTCTCAGGTCCGTGTGAGAC-3′

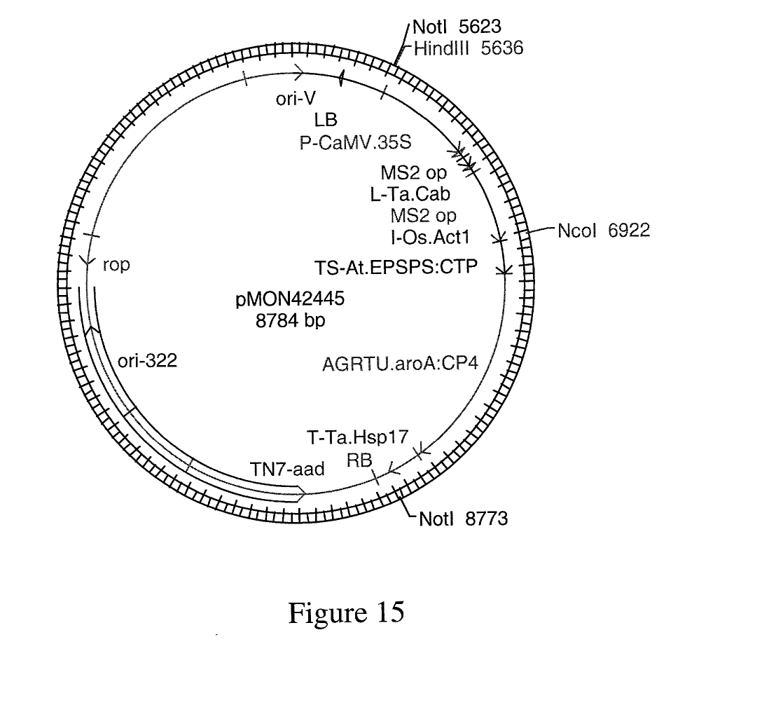

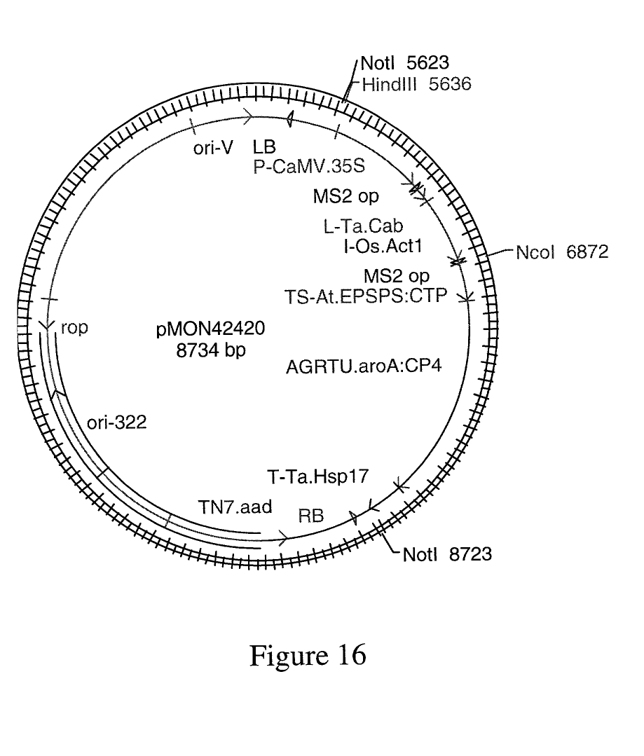

These primers are used to amplify a 789 bp fragment containing the MS2 operator sequence and the P-CaMV.35S promoter from pMON42410. The PCR product is digested with HindIII and SmaI and cloned back into pMON42410, resulting in construct pMON42444, which contained a single MS2 operator. The second operator is taken from pMON42426 by HindIII/XbaI digestion. This insert is ligated into pMON42444 (HindIII/XbaI digest) to generate pMON42445 (P-CaMV.35S/MS2op/L-Ta.Cab/MS2op/I-Os.Act l/TS-At.EPSPS :CTP/T-Ta.Hsp 17) (FIG. 15), which contained two MS2 operators. This vector construction placed the second operator between the leader sequence and the intron sequence in the DNA construct. Alternatively, the second operator can be located between the intron and the start of translation of the gene of interest in the DNA construct. Processing of the intron during expression of the either gene cassette results in the operators flanking the leader sequence. This construction in illustrated in pMON42420 (FIG. 16) (P-CaMV.35S/MS2 op/L-Ta.Cab/I-Os.Act1/MS2 op/TS-At.EPSPS :CTP/AGRTU.aroA:CP4).

Translational Repression of Reporter Genes Containing Two Translational Operators.

The MS2 CP may repress the translation of a target mRNA through steric hindrance. Binding of the MS2 CP to the 5′-UTR may interfere with the translation initiation and/or ribosomal scanning. Translation of a reporter mRNA containing more than one op may be more efficiently repressed than that containing only a single op. pMON42445 (FIG. 15) is constructed to test this possibility in corn protoplasts by transient assay as described in Example 2. pMON42445 contains two MS2 translational operators, one located 19 nucleotides 3′ to the end of the mRNA and another 10 nucleotides 3′ to the wheat cab leader (L-Ta.Cab) sequence. The results in Table 10 indicate that translation of the CP4 EPSPS MRNA containing two MS2 operators is more efficiently repressed than mRNA containing only one MS2 operator.

TABLE 10
Translational repression of CP4 EPSPS mRNA with double MS2 op

| | | CP4 | | |
|---|---|---|---|---|
| Treatment | pMON DNA | Reading^{b} | STDER | % Expression |
| 1 | 42444 | 0.461 | 0.089 | 100.0 |
| 2 | 42444 + 42180(1:1)^{a} | 0.257 | 0.023 | 55.7 |
| 3 | 42445 | 0.484 | 0.025 | 100.0 |
| 4 | 42445 + 42180(1:1) | 0.163 | 0.013 | 33.7 |
| 5 | 42445 + 42180(1:2) | 0.089 | 0.015 | 18.4 |

^{a}The molar ratio of the target and MS2 plasmid DNA
^{b}Mean of four independent electroporations

### Example 5

### Cloning of the Yeast Ribosomal Protein RPL32 Gene

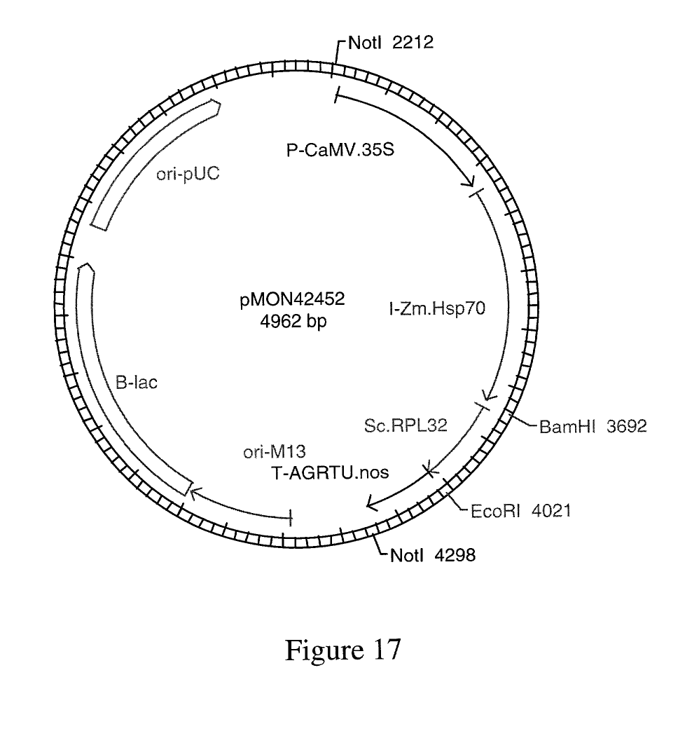

The yeast (Saccharomyces cerevisiae) ribosomal protein RPL32 is an RNA-binding protein that, when bound to its operator, can repress its own translation (Dabeva et al., J. Biol. Chem. 268:19669-19674 (1993)). The Sc.Rp132 gene is cloned into a plant expression cassette pMON42452 (P-CaMV.35S/I-Zm.Hsp70/Sc.Rp132/T-AGRTU.nos) (FIG. 17) in order to demonstrate its ability to repress expression of an operator/reporter gene in corn protoplasts. Two primers are designed based on published sequence (Genbank # z72552) to amplify the gene from yeast:

JHRPL32U (SEQ ID NO:24)
5′-GCG CGG ATC CAA CAA TGG CCC CAG TTA AAT CCC AAG
AAT C-3′
JHRPL32L (SEQ ID NO:25)
5′-GCG CGA ATT CCT TAA GCC AAG GTG GTC AAG ATA
TC-3′

Restriction sites (underlined) are incorporated in the primers to facilitate cloning. One μL of MONY7 yeast cells is used directly as the source of DNA template for PCR amplification, which is carried out with the ExpandTm High Fidelity PCR system (Roche Boehriger Mannheim, Nutley, N.J.; Cat. # 1732641). The PCR parameters are step 1: 94° C., 5 min; step 2: 94° C., 20 seconds; step 3: 60° C., 30 seconds; step 4: 72° C., 45 seconds; step 5: 30 cycles to step 2; step 6: 72° C., 5 min; step 7: end.

The amplified Sc.Rpl32 gene fragment is digested with BamHI and EcoRl and cloned into pMON42179, resulting in pMON42452 (FIG. 17). The entire coding region of the Sc.Rpl32 gene is sequenced and confirmed to be identical to the published Sc.Rpl32 gene sequence (Genbank #z72552).

A version of the gene enhanced for plant expression (Genbank #z72552) is constructed that has codon usage typical of Zea mays plant genes. The codon-optimized coding sequence is obtained by reverse translation using the corn codon usage table according to the GCG protocol (SEQLAB GCG, Madison, Wis.), whereas the native amino acid sequence of the RPL32 (Genbank #z72552) is retained. The Zea mays codon usage table of Nakamura (http://www.dna.affrc.gojp/˜nakamura/) is an additional source of codon usage information from which a gene sequence can be constructed for enhanced expression in monocot plants. The GC content of the Sc.Rpl32 gene is changed from 41.8% to 62.9%. The enhanced plant expression Sc.Rpl32 gene is generated from 10 oligonucleotides by SOE PCR (Horton et al., Gene 77:61-68 (1989); Yon et al., Nucl. Acids Res., 17:4894 (1989)). Oligonucleotides are purchased from Gibco BRL (Gaithersburg, Md.). The sequence of the Sc.Rpl32 gene as modified for enhanced expression in monocots is shown in SEQ ID NO:26. This Sc.Rpl32:en gene with attached BamHI and EcoRI linkers replaces the Sc.Rpl32 wild type gene sequence in pMON42452 for enhanced expression in monocot plants.

Reporter Plasmid for the RPL32 Protein.

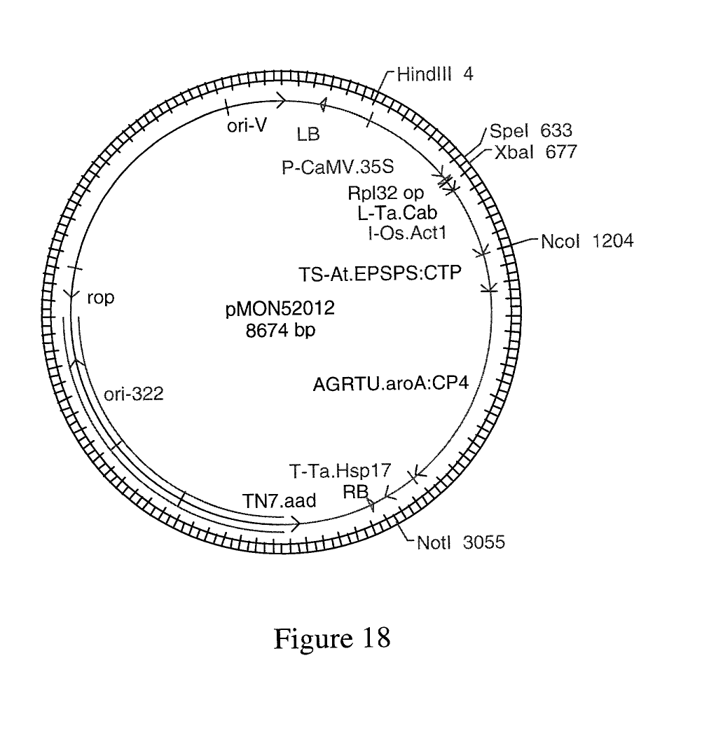

The RPL32 binding sequence or operator consists of more than 70 nucleotides; however, an internal deletion mutant named miniL32RNA maintains the function of the wild-type operator (Li et al., J. Mol. Biol. 250:447-459 (1995)). The miniL32RNA operator is inserted into the 5′-UTR of the CP4 EPSPS reporter gene in pMON42410 via PCR, resulting in pMON52012 (FIG. 18). The primers used for the PCR are

JHMINI32L (SEQ ID NO:27):
5′-CGC GTC TAG ATC GAC GAT CTC TGC GTA TAA AAC ACT CCC GTC TCC
CTT ACT AGT ACG TCC TCT CC-3′
35S5′(SEQ ID NO:22):
5′-GTT AAC AAG CTT CTG GAG GTC CGA TGT GAG AC-3′

The underlined sequence in primer JHMINI32L is complementary to the Sc.Rpl32 operator coding sequence. The amplified fragment, which contains the Sc.Rpl32 operator, is cloned into pMON42410 in the 5′ to 3′ orientation, resulting in pMON52012 (FIG. 18). The operator in pMON52012 is confirmed to be correct by a PCR DNA sequencing method (ABI Prism™ 377, Rerkin Elmer, Foster, Calif.).

Yeast RPL32 Represses Translation of Transgene in Corn Protoplasts.

pMON52012 (FIG. 18) is electroporated into corn protoplasts either alone or together with pMON42452. Table 11 summarizes the results of a typical assay and shows that the yeast RPL32 protein also repressed the expression of the CP4 EPSPS reporter gene when the operator is incorporated into its 5′-UTR. Translation is reduced up to 43% of the control, or up to 2.3-fold. RPL32 did not repress the expression of a CP4 EPSPS gene that does not contain the operator in its 5′-UTR, indicating that the RPL32 mediated repression of gene expression resulted from the specific interaction of RPL32 and its cognate operator.

TABLE 11
RPL32 represses CP4 EPSPS expression in corn protoplasts

| | | CP4 | | |
|---|---|---|---|---|
| Treatment | pMON DNA | Reading^{b} | STDER | % Expression |
| 1 | 52012 | 0.79 | 0.162 | 100.0 |
| 2 | 52012 + 42452 (1:2)^{a} | 0.54 | 0.056 | 68.3 |
| 3 | 52012 + 42452 (1:15) | 0.34 | 0.078 | 43.0 |
| 4 | 42410 | 0.25 | 0.040 | 100.0 |
| 5 | 42410 + 42452 (1:15) | 0.56 | 0.062 | 224.0 |

^{b}The DNA molar ratio of the target plasmid to coat protein plasmid.
^{a}Mean of four independent electroporations.

### Example 6

### Optimal Placement of MS2 Operator in the 5′UTR Increases Translational Repression Efficiency

The distance from the operator to the transcription initiation or the 5′ end of the target mRNA has a significant effect on the translational repression efficiency in several translational repression systems. For example, for an IRE-IRP complex to efficiently inhibit protein synthesis, the IRE operator must be localized in a proximal position of the mRNA (Goossen et al., EMBO J. 9:4127-4133 (1990); Goossen et al., Mol. Cell. Biol. 12:1959-1966 (1992)). An increase in distance between the MS2 operator and the 5′ end of the mRNA showed a negative effect on efficiency of translational repression (Stripecke et al., NAR 20:5555-5564 (1992)).

A series of constructs is made in which the distance from the 5′ end of the mRNA transcript to the MS2 operator ranged from 37 nucleotides (nt) to 4 nt (Table 12). These constructs are tested in corn protoplasts to determine the effect of the position of the MS2 operator in the 5′UTR on the efficiency of translational repression. Table 12 shows that there is a significant increase in the repression efficiency if the distance from the operator to the 5′ end of the mRNA is reduced from 37 nt to 19 nt. A further decrease in the distance did not cause any additional increase in translational repression efficiency.

TABLE 12
The efficiency of translational repression by the MS2
coat protein/operator is position-dependent

| | pMON | CP4 | | % | hop |
|---|---|---|---|---|---|
| Treatment | DNA^{a} | Reading^{b} | STD | Expression | Location |
| 1 | 42410 + 42180 | 0.291 | 0.016 | 100 | no |
| | | | | | operator |
| 2 | 42424 + 42180 | 0.156 | 0.018 | 53.6 | 37 nt from |
| | | | | | 5′ end |
| 3 | 42426 + 42180 | 0.025 | 0.003 | 8.6 | 19 nt from |
| | | | | | 5′ end |
| 4 | 42429 + 42180 | 0.025 | 0.002 | 8.6 | 14 nt from |
| | | | | | 5′ end |
| 5 | 42430 + 42180 | 0.02 | 0.002 | 6.9 | 9 nt from |
| | | | | | 5′ end |
| 6 | 42431 + 42180 | 0.026 | 0.001 | 8.6 | 4 nt from |
| | | | | | 5′ end |

^{a}Themolar ratio of the target and MS2 plasmid DNA is 1:1
^{b}Mean of four independent electroporations

### Example 7

### Translational Repression by MS2 Fusion Proteins

pMON52035 (FIG. 4), which encodes the MS2 coat protein fusion (MS2 dimers), is tested for translational repression in corn protoplasts. Table 13 shows the result of a typical assay.

TABLE 13
Translational repression by MS2 CP dimers

| | | CP4 | | |
|---|---|---|---|---|
| Treatment | pMON DNA | Reading^{b} | STDER | % expression |
| 1 | 42916 | 0.137 | 0.021 | 100.0 |
| 2 | 42916 + 42180 (1:1)^{a} | 0.067 | 0.015 | 48.9 |
| 3 | 42916 + 42180 (1:2) | 0.036 | 0.012 | 26.3 |
| 4 | 42916 + 52035 (1:1) | 0.033 | 0.007 | 24.1 |
| 5 | 42916 + 52035 (1:2) | 0.030 | 0.009 | 21.9 |

^{a}The molar ratio of the target and MS2 plasmid DNA is 1:1 or 1:2
^{b}Mean of four independent electroporations

The results indicated that the MS2 fusion protein is as active as the monomer in the transient assay. Because the dimer is more stable than the monomer, it is potentially more efficient in translational repression in vivo.

### Example 8

### MS2 Translation Repression System in Transgenic Arabidopsis

Transformation and Analysis of Arabidopsis Plants.

Arabidopsis plants are transformed by the Agrobacterium infiltration method as described (Bechtold et al., Methods Mol. Biol. 82:259-66 (1992)). DNA gel blots (Southern blot analysis), RNA gel blots (northern blot analysis) and protein immunoblots (western blot analysis) are carried out using standard protocols (Sambrook et al., Molecular Cloning: A laboratory manual, 2^{nd} ed. Cold Spring Harbor Laboratory Press, Cold Spring Harbor, N.Y., (1989)).

MS2 Translation Repression System in Transgenic Arabidopsis.

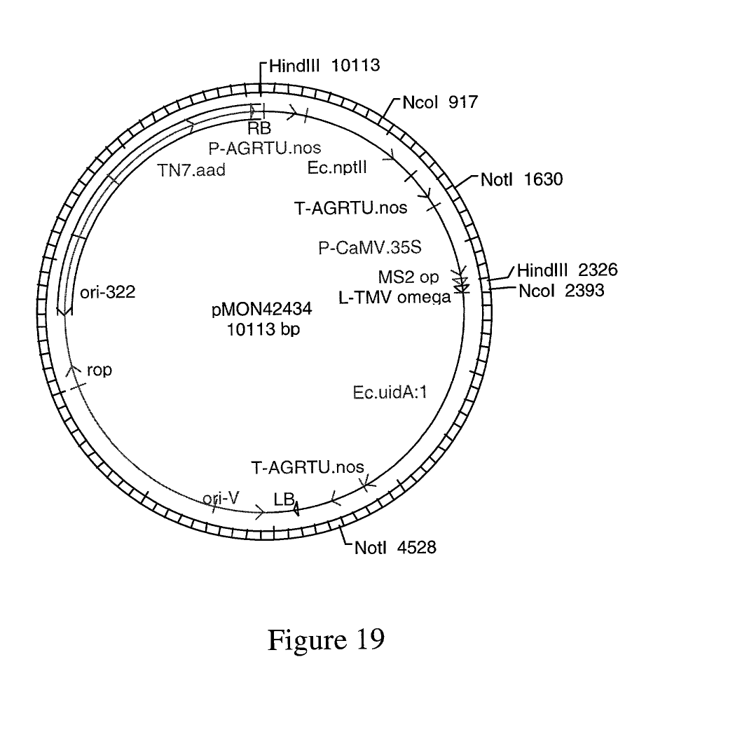

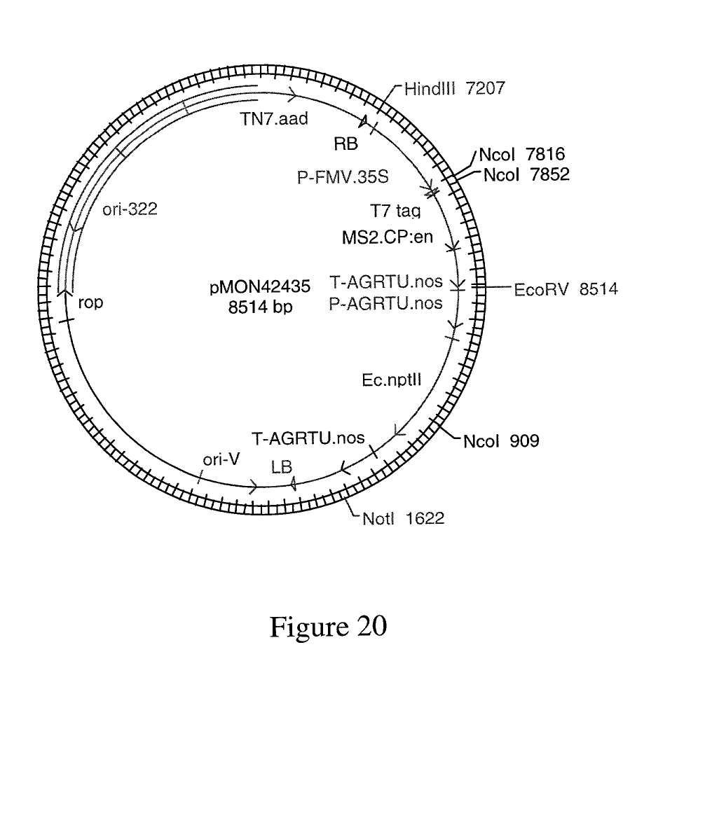

Two constructs are made to demonstrate that MS2 coat protein mediates translational repression in planta. pMON42434 (FIG. 19) is a reporter plasmid with uidA as the reporter gene and pMON42435 (FIG. 20) is a MS2 coat protein expression plasmid. V1 plants positive for GUS and MS2 coat protein are identified using a GUS assay (Jefferson et al., Plant Mol. Bio. Rep. 5:387-405 (1987)) and western blot analysis, respectively. Positive plants with a low copy number are identified by Southern analysis then cross pollinated. F1 plants from the cross are then subjected to PCR analysis to identify segregants that contained both transgenes as well as siblings that contained only the uidA gene. GUS expression levels are determined by MUG assays (Jefferson et al., Plant Mol. Bio. Rep. 5:387-405, (1987)) in both groups of plants for three separate crosses (25×1, 25×12, and 14×23). Table 14 shows that GUS activity is reduced to 6.9% of the control, or 14-fold repression, in plants containing the MS2 coat protein. Western analysis confirmed that MS2 coat protein gene expression is required for repression of GUS expression in these plants. Most significant, the efficiency of MS2 coat protein-mediated repression of GUS expression in planta is similar to that in the protoplast transient assay. Northern blot analysis showed that the amount of steady state uidA gene transcript remained similar in segregating siblings as compared to those containing only the uidA gene or both the uidA gene and the MS2 coat protein gene (Table 14), confirming that repression is at the translational level.

TABLE 14
MS2 coat protein represses GUS expression in Arabidopsis

| | Segregant | uidA | GUS | CP | % GUS |
|---|---|---|---|---|---|
| Cross | Genotype | RNA^{d} | Activity | Expression^{c} | Expression |
| Line 25 × | uidA | +++++ | 61.42 | — | 100.0 |
| Line 1 | uidA, CP^{a} | +++++ | 9.75 | ++++ | 15.9 |
| Line 25 × | uidA | ++ | 3.78 | ND^{b} | 100 |
| Line 12 | uidA, CP | ++ | 0.26 | ++ | 6.9 |
| Line 1 × | uidA | +++++ | 80.77 | ND | 100.0 |
| Line 23 | uidA, CP | +++++ | 13.74 | ++ | 17.0 |

^{a}CP = MS2 coat protein gene
^{b}ND = not determined
^{c}relative band intensity by western analysis
^{d}relative band intensity by northern analysis

### Example 9

### Transformed Corn Plants Containing the Ms2 Translation Repression System

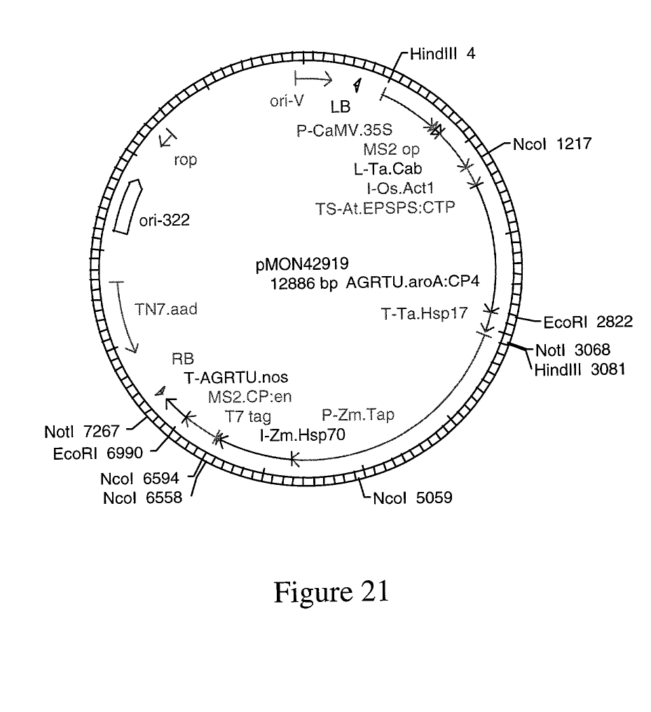

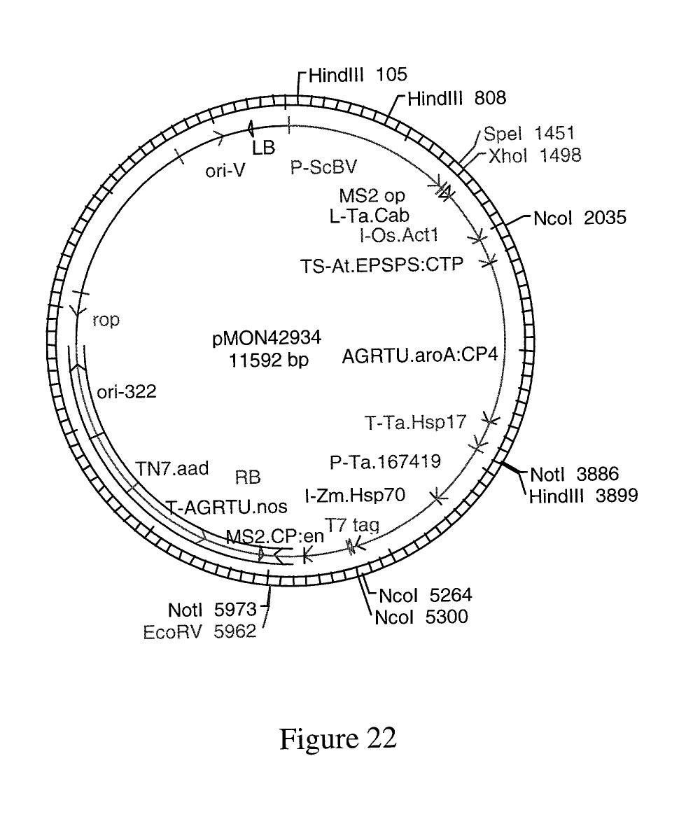

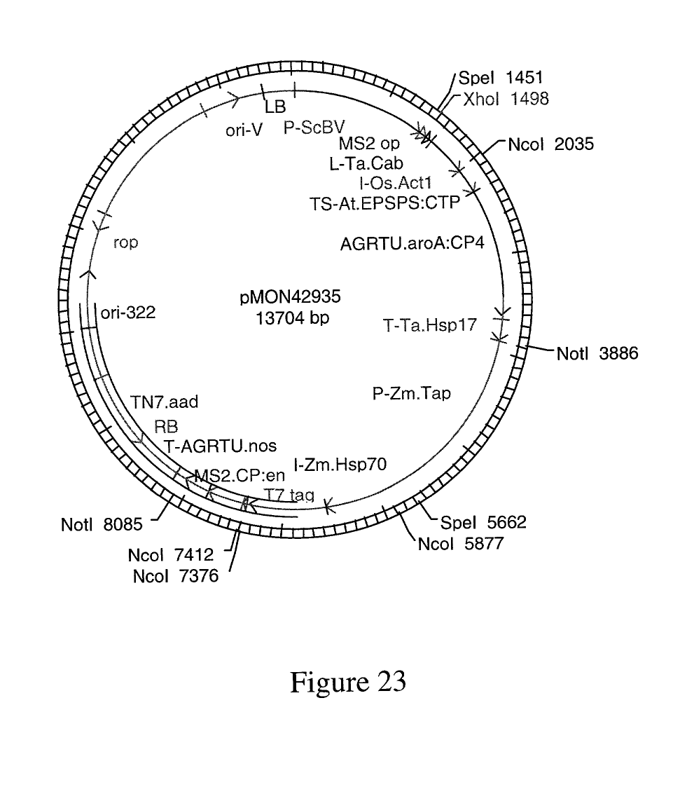

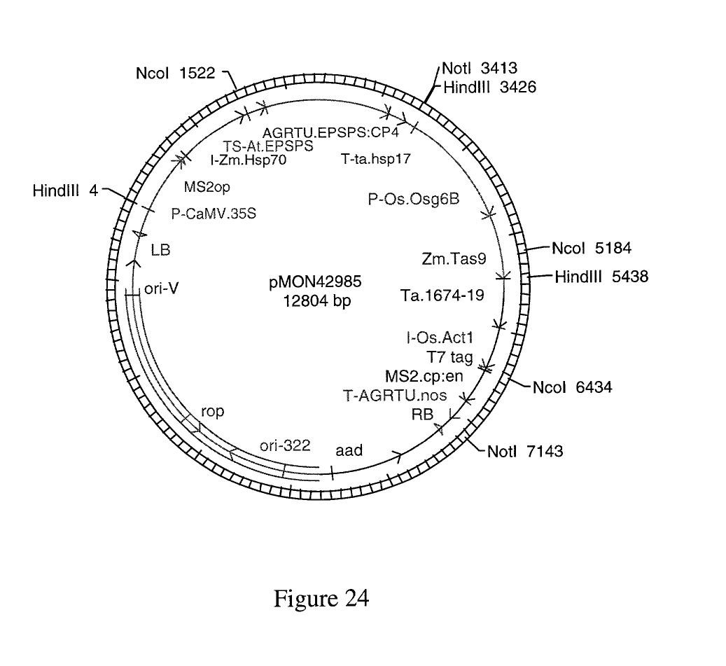

Transgenic corn containing a MS2 CP-expressing plasmid pMON42940 (P-CaMV.35S/I-Zm.Hsp70/MS2.CP:en/T-AGRTU.nos) or a reporter plasmid pMON52027 (P-Os.Act1/I-Os.Act 1/TS-At.EPSPS :CTP/MS2op/AGRTU.aroA:CP4/T-Ta.Hsp 17) is produced by bombarding embryogenic corn tissue culture cells using a biolistic particle gun as described in U.S. Pat. No. 5,424,412. Transformed cells are selected for glyphosate tolerance and whole plants are regenerated and grown under greenhouse conditions. In addition, freshly isolated Type II immature HiIIxLH198 and HiII corn embryos are inoculated with Agrobacterium containing pMON42919 (P-CaMV:35S/MS2 op/L-Ta.Cab/I-Os.Act1/TS-At.EPSPS :CTP/AGRTU.aroA:CP4/T-Ta.Hsp 17::P-Zm.Tap/I-Zm.Hsp70/T7 tag-MS2.CP:en/T.AGRTU.nos) (FIG. 21); pMON42934 (P-ScBV/MS2 op/L-Ta.Cab/I-Os.Act 1/TS-At.EPSPS :CTP/AGRTU.aroA:CP4/T-Ta.Hsp 17: :P-1674-19/I-Zm.Hsp7O/T7 tag-MS2.CP:en/T-AGRTU.nos) (FIG. 22); pMON42935 (P-ScBV/MS2op/L-Ta.Cab/I-Os.Act1/TS-At.EPSPS :CTP/AGRTU.aroA:CP4/T-Ta.Hsp 17:: P-Zm.Tap/I-Zm.Hsp70/T7 tag-MS2.CP:en/T-AGRTU.nos) (FIG. 23); or pMON42985 (P-CaMV.35S/MS2op/I-Zm.Hsp70/TS-At.EPSPS :CTP/AGRTU.aroA:CP4/T-Ta.Hsp 17::P-Os.Osg6B-Zm.Tas9-Ta. 1674-19/1-Os.Actl/T7tag-MS2.CP:en/T-AGRTU.nos) (FIG. 24) and co-cultured 2-3 days in the dark at 23° C. The embryos are then transferred to delay media (N6 1-100-12/micro/Carb 500/20 μM AgNO₃) and incubated at 28° C. for 4 to 5 days. All subsequent cultures are kept at this temperature. Coleoptiles are removed one week after inoculation. The embryos are transferred to the first selection medium (N6 1-0-12/Carb 500/0.5 mM glyphosate (not formulated Roundup®)). Two weeks later, surviving tissue is transferred to the second selection medium (N6 1-0-12/Carb 500/1.0 mM glyphosate). Surviving callus is subcultured on 1.0 mM glyphosate every two weeks for a total of three subcultures until transformed plants could be identified. Once events are identified, the callus tissue is transferred to the regeneration medium (MS 0. ID 0.1 μM ABA) and incubated for two weeks. The regenerating calli are transferred to a high sucrose medium and incubated for two weeks. Plantlets are then transferred to MMSOC (4.3g Gibco MS, 1.95g MES, 2.OmL MMS Vitamins, 40.0 g maltose, 2g Schweizer Hall, after autoclaving add 2 mL ascorbic acid, glyphosate and adjust the pH to 5.8 with KOH) media in phytatrays and kept for two weeks. Plants with roots are transferred to soil. The above plant expression vectors contain elements that enhance expression of the MS2 CP in the male tissues of plants.

Evaluation of transgenic corn plants. Three RO plants are regenerated for any given transgenic event. These three plants are expected to be nearly isogenic, because they are thought to be derived from a single transgenic plant cell. Thus, one plant is used as a non-sprayed control and the remaining two plants are treated with glyphosate (formulated Roundup®) at the V5-V8 stage by means of a linear track sprayer set to deliver glyphosate at a rate of 64 ounces/acre (oz/A) (4.75 L/hectare). Vegetative tolerance to the glyphosate is visually evaluated one week after spraying based on a scale of 0 to 5 (0=no observable/vegetative effect of glyphosate; 1=chlorosis observed; 2=advanced chlorosis, minor necrosis; 3=advanced chlorosis, moderate necrosis; 4=advanced chlorosis, severe necrosis; 5=no live tissue remaining). All R0 plants, treated or non-treated, are isolated during anthesis with bags to prevent outcrossing. Next, reciprocal crosses are performed on each of the three plants with a non-transgenic parent. The number of seeds is scored for each cross. For both sprayed and non-sprayed plants, when the transgenic plant is used as the pollen recipient, the number of seeds served as a measure of female fertility. Likewise, when the transgenic plant is used as pollen donor, the number of seeds served as a measure of its male fertility. Percent male fertility is then estimated as the seed number of the sprayed plant divided by the seed number of its non-sprayed near-isogenic plant when the transgenic plants are used as the pollen donor. Three R0 events from three independent constructs showed 84-100% male sterility with apparently good female fertility (Table 15).

TABLE 15
Glyphosate-induced male sterility in corn

| | R0 | | # Seeds: | # Seeds: | |
|---|---|---|---|---|---|
| | Plant | | As Pollen | As Pollen | % Male |
| pMON | Number | Treatment | Recipient | Donor | Sterility |
| 42919 | S16755 | No glyphosate | ND | 126 | 0 |
| | S16756 | 64 oz/A glyphosate | 127 | 6 | 95 |
| | S16757 | 64 oz/A glyphosate | 148 | 20 | 84 |
| 42934 | S17190 | No glyphosate | 190 | 149 | 0 |
| | S17194 | 64 oz/A glyphosate | 220 | 8 | 95 |
| | S17195 | 64 oz/A glyphosate | 172 | 0 | 100 |
| 42935 | S17283 | No glyphosate | 248 | 167 | 0 |
| | S17287 | 64 oz/A glyphosate | 136 | 7 | 96 |
| | S17288 | 64 oz/A glyphosate | ND | 0 | 100 |

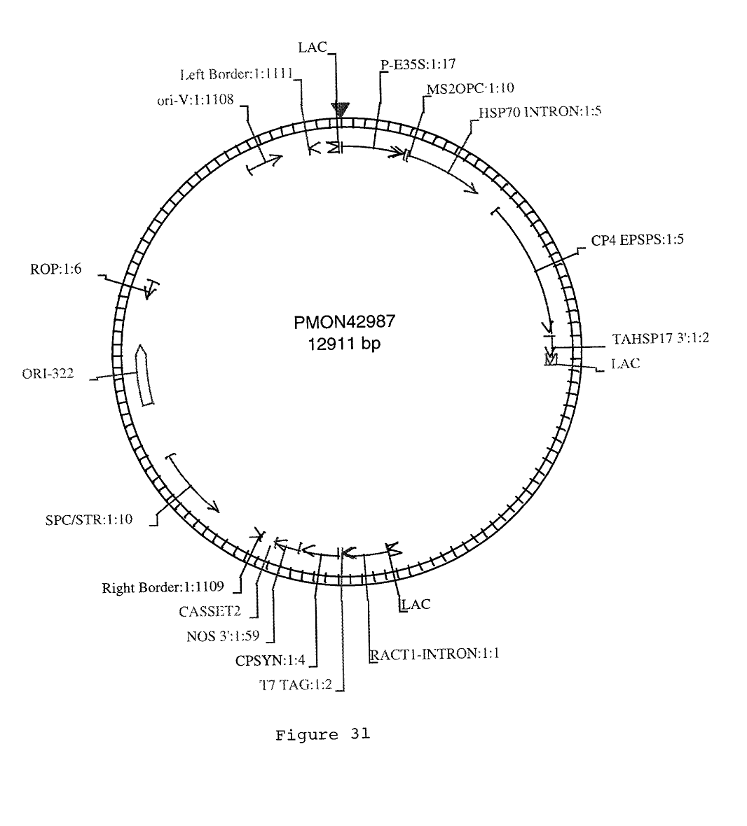

Because the MS2 CP represses translation in a dose-dependent fashion, it is expected that an increase in the ratio of MS2 CP to CP4 EPSPS will result in more efficient translational repression. Construct pMON42987 (FIG. 31) (P-CaMV:35S/I-Zm.Hsp70/MS2 op/C21/TS-At.EPSPS :CTP/T-Ta.Hsp 17 3′::lox-p/P-Zm.Tap/T7tag-MS2 Synthetic/T-AGRTU.nos/loxp) was made which expresses less EPSPS in the male reproductive cells. The lower expression of EPSPS results in a higher molar ration of MS2 CP to EPSPS mRNA molecules in the anther, which in turn is expected to result in more efficient translational repression, hence glyphosate-induced male sterility. Table 16 shows that 84% of the RO events were completely male sterile when sprayed with glyphosate at 64 oz/A. Seed setting in these plants was comparable to that in the unsprayed controls. Note that the MS2 CP expression cassette in pMON42987 is flanked by two lox-p sequences. When a pMON42987 plant is crossed with a plant expressing Cre, the MS2 CP cassette will be excised and the resulting progeny is expected to have higher male fertility when sprayed with glyphosate.

TABLE 16
Glyphosate-induced male sterility in pMON42987 R0 corn plants

| | # Male | # Male | % Male | |
|---|---|---|---|---|
| | Sterile | Fertile | Sterile | Average |
| Treatment | Plant | Plant | Plants | Seed/plant |
| Gly-sprayed | 27 | 5 | 84 | 52.7 (±9.6) |
| 64oz/A | | | | |
| Un-sprayed | 0 | 12 | 0 | 76.6 (±12.5) |

Evaluation of MS2 Translation Repression System Transgenic Lines Under Field Conditions.

Field tests are conducted to evaluate the vegetative tolerance and male sterility of transgenic corn lines selected from the greenhouse tests. Lines for the field trial are selected based on previous scores showing good to excellent vegetative tolerance, very high or complete male sterility induced by glyphosate, and available seed supply. The pedigree of plants in the field test is either [(Ro×B73)×B73] or (B73×Ro). Based on these pedigrees, it is expected that half of the plants in a plot would be killed by glyphosate (formulated Roundup®) application, because they would not contain the transgene.

The field test is set up in two blocks, each block containing four groups of plots that would receive a single spray treatment. The treatments are (1) no spray, (2) 32 oz/acre Roundup® at the 1-2 leaf stage, (3) 32 oz/acre Roundup® at the 4-5 leaf stage, and (4) 32 oz/acre Roundup® at the 6-8 leaf stage. Plants are scored for male fertility when they began to flower, and scoring is repeated every 1-2 days for about eight days. Observations included the length of time between emergence of the tassel from the first plants and from the last plant in each plot. A sample of the anthers is collected from each plant, and the viability of pollen is judged with the aid of a hand-held microscope.

Glyphosate sprays at the 6-8 leaf stage are found to be very effective for producing male sterility. No or very few anthers are visible on plants sprayed at the 6-8 leaf stage by the time all plants in the respective unsprayed plots had begun to flower. Plants that are male-sterile from the Roundup® treatment are pollinated with B73 pollen. Seed set from ears borne on treated plants is normal, indicating full female fertility.

Backcross Conversion of the Translation Repression System (TRS) into Elite Inbreds/Varieties.

The breeding strategy described in Table 17 is applicable for all sexually compatible crop and ornamental plants. Transgenic plants containing the TRS are used as the female parent in each backcross to the recurrent parent generation if plants containing TRS are identified by glyphosate application, causing male sterility. In the selfing generations, plants containing TRS need to be male fertile in order to self-pollinate; therefore, they must be identified by a glyphosate application at such a time that the application does not cause male sterility; by molecular methods such as DNA based assays (PCR, Southern blotting); or by protein based assays (ELISA, Western blotting).

TABLE 17
Backcross conversion

| Gen | Activity¹ | Comments | % RP² |
|---|---|---|---|
| 1 | Donor × RP | id TRS plants via gly spraying, | 50 |
| | | molecular method | |
| 2 | F1 × RP | id TRS plants via gly spraying, | 75 |
| | | molecular method | |
| 3 | BC1 × RP | id TRS plants via gly spraying, | 87.5 |
| | | molecular method | |
| 4 | BC2 × RP | id TRS plants via gly spraying, | 93.75 |
| | | molecular method | |
| 5 | BC3 × RP | id TRS plants via gly spraying, | 96.88 |
| | | molecular method | |
| 6 | BC4 × RP | id TRS plants via gly spraying, | 98.44 |
| | | molecular method | |
| 7 | BC5 × RP | id TRS plants via gly spraying, | 99.22 |
| | | molecular method | |
| 8 | BC6→BC6S1 | Self BC6 to begin fixing TRS in | |
| | | homozygous state | |
| 9 | BC6S1→BC6S2 | Self BC6S1 to generate fixed S2 | |
| | | families | |
| 10 | BC6S2→BC6S3 | Identify homozygous S2 positive | |
| | | families and increase | |

¹Donor parent is the line or plant that contains TRS, RP (recurrent parent) is the elite inbred line/variety,
F1 is the generation resulting from the first cross, BCn represents the n^{th} backcross generation, BCnSn represents
the n^{th} backcross and n^{th} selfed generation.
²Theoretical recovery of the recurrent parent in the harvested generation

Seed Increase (Maintenance) of an Inbred Line or Variety Containing TRS:

In order to increase the seed or maintain an inbred/variety that is homozygous for TRS in an isolated field, the plants are grown without glyphosate application and allowed to open pollinate (cross pollinated crop) or self pollinate (self pollinated crop).

Alternatively, an inbred/variety that is homozygous for TRS is physically isolated from other corn plants. Glyphosate is applied (e.g., 8 oz to 32 oz/A at the 2-4 leaf stage) such that negative plants are killed, thereby maintaining the TRS genotypic and transgene purity. In this way, weed control is improved but the male fertility of the plants is not reduced to the point that yields are economically decreased. One or more applications of glyphosate can be made without inducing male sterility.

As a further alternative, an inbred/variety that is homozygous for TRS is planted in the field physically isolated from other corn plants. Glyphosate is applied on rows that are designated as female rows such that the plants are rendered male sterile, but not on rows designated as male rows. Seed is harvested from female rows only. This method is useful for ensuring out-crossing during seed increase and/or if the window of glyphosate application is such that it is not possible to apply glyphosate without substantially decreasing male fertility, assuming that seed increases from crossing are economical.

Hybrid Seed Production Via Male Sterility, Resulting in a Glyphosate-tolerant Crop.

In a physically isolated field, female rows containing an inbred/variety that is homozygous for TRS are planted. In the same field, male rows that are homozygous for a transgenic event that confers commercial levels of glyphosate tolerance (e.g., NK603) are also planted. Glyphosate is applied over the female and male rows in such a manner (e.g., 16-64 oz/acre, V4-V10 vegetative growth stage) that the female rows are male sterile but vegetatively tolerant to the glyphosate application. Because of the commercial level of glyphosate tolerance in the male inbred/variety, the male parent will be male fertile and vegetatively tolerant. Harvest seed from female rows only. The resulting F1 hybrid seed is hemizygous for the commercial glyphosate tolerance allele and TRS. Because F1 plants contain one copy of the commercial glyphosate tolerance allele, the plants will be fully tolerant to glyphosate and will also be fully male fertile when applications of glyphosate are made to the F1 generation. Seed production utilizing this system results in a higher frequency of F1 plants that are vegetatively tolerant to glyphosate than when only one parent contains a commercial glyphosate tolerance allele, providing an advantage over the current practice of producing glyphosate tolerant corn hybrids when only one parent contains the commercial glyphosate tolerance allele.

### Example 10

### Transformed Wheat with the MS2 Translation Repression System

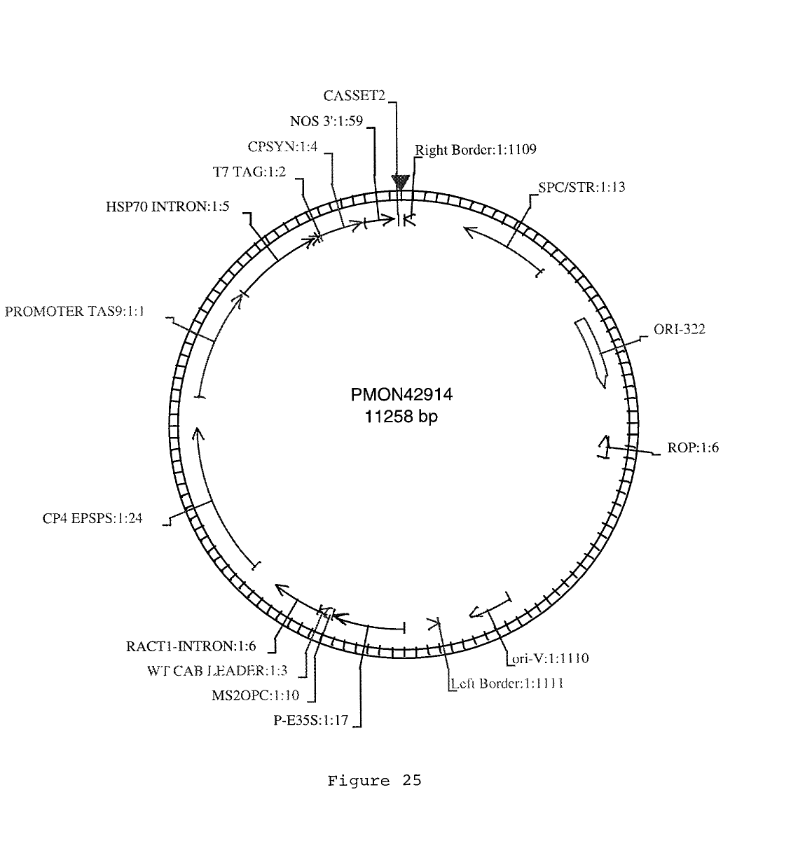

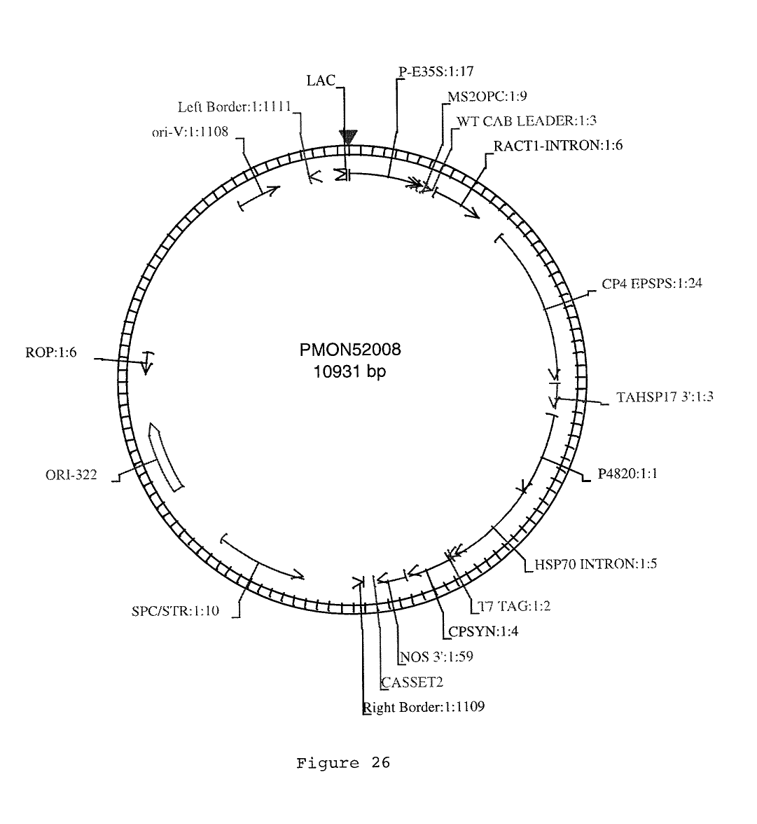

Two plant expression vectors are constructed for transformation of wheat and the expression of the TRS for male sterility and hybrid seed production in wheat. The pollen-specific promoter isolated from corn tassel genomic library by Genome Walker™ is amplified by PCR as a Hind III-Bgl II fragment and designated as P-Zm.Tas9. The DNA fragment is digested with Hind III and Bgl II, and ligated into pMON42180 (FIG. 3) that has been digested with the same enzymes and the resulting plasmid is pMON42183. A Not I fragment consisting of P-Zm.Tas9/I-Zm.Hsp70/MS2.CP/T-AGRTU.nos is isolated from pMON42183 and ligated into pMON42426 (FIG. 6) at the same site, resulting in pMON42914 (FIG. 25) (P-Zm.Tas9/I-Zm.Hsp70/MS2.CP/T-AGRTU.nos). pMON52008 (FIG. 26) (P-CaMV:35S/MS2op/L-Ta.Cab/I-Os.Actl/TS-At.EPSPS/AGRTU.aroA:CP4/T-Ta.Hsp 17: :P-700354820/1-Zm.Hsp70/MS2.CP:en/T-AGRTU.nos) is similar to pMON42914 except that a corn anther-specific promoter P-Zm.700354820 that is isolated by Genome WalkerM from a corn anther genomic DNA library replaces P-Zm.Tas9.

Transformation of Wheat with the Translation Repression System.

Immature embryos of wheat (Triticum aestivum L) cv Bobwhite are isolated from the immature caryopsis 13-15 days after pollination and cultured on CM4C (Table 18) for 3-4 days. Embryos showing active cell division but no apparent callus formation are selected for Agrobacterium infection.

TABLE 18
Supplemental Components in Basal Media¹

| Components | CM4 | CM4C | MMS.2C | MMS0 |
|---|---|---|---|---|
| 2,4-D (mg/L) | 0.5 | 0.5 | 0.2 | — |
| Pichioram (mg/L)² | | 2.2 | 2.2 | |
| Maltose (g/L) | 40.0 | 40.0 | 40.0 | 40.0 |
| Glutamine (g/L) | | 0.5 | 0.5 | |
| Magnesium Chloride (g/L) | | 0.75 | 0.7 | |
| Casein Hydrolysate (g/L) | | 0.1 | 0.1 | |
| MES (g/L) | | 1.95 | 1.95 | 1.95 |
| Ascorbic Acid (mg/L)² | | 100.0 | 100.0 | 100.0 |
| Gelling Agent (g/L)³ | 2(P) | 2(P) | 2(G) | 2(G) |

¹All media contained MS (Murishige and Skoog) basal salts and MS vitamins. The pH in each medium is adjusted to 5.8.
²Filter-sterilized and added to the medium after autoclaving.
³Phytagel ™ (P) or Gelrite ®(G).

A disarmed Agrobacterium strain C58 (ABI) harboring a binary vector of interest (pMON42914 and pMON52008) is used for all the experiments. Cultures of Agrobacterium are initiated from glycerol stocks or from a freshly streaked plate and grown overnight at 26° C.-28° C. with shaking (approximately 150 rpm) to mid-log phase (OD₆₆₀=1-1.5) in liquid LB medium, pH 7.0 containing 50 mg/L kanamycin, 50 mg/L streptomycin and spectinomycin and 25 mg/L chloramphenicol with 200M acetosyringone (AS). The Agrobacterium cells are resuspended in the inoculation medium (liquid CM4C) and the density is adjusted to OD₆₆₀ of 1. Immature embryos cultured in CM4C medium are transferred to sterile petri plates (16×20 mm) or wells of a 6-well cell culture plate (Costar Corporation, Cambridge, Mass.) containing 10 mL of inoculation medium per petri plate or 5 mL per cell culture cluster plate. An equal amount of the Agrobacterium cell suspension is added, such that the final concentration of Agrobacterium cells is an OD₆₀₀ of 0.5. In most experiments, pluronic F68 is added to the inoculation mixture at a final concentration of 0.01%. The ratio between the Agrobacterium and immature embryos (IEs) is about 10 mL: 20-200 IEs. The inoculation is allowed to proceed at 23° C.-26° C. for 5-60 minutes.

After the inoculation period, the remaining Agrobacterium cells are removed from the explants using the in-house vacuum equipment. A piece of sterile Whatman No. 1 filter paper (to fit the size of the petri plate) is placed in each of 60×15 or 60×20 mm petri dishes. Two hundred microliters of sterile water is placed in the middle of the filter paper. After 2-3 minutes, the inoculated immature embryos are placed in the plates. Usually 20-50 explants are grouped as one stack (about 1 cm in size and 60-80 mg/stack), with 4-5 stacks on each plate. The plates are immediately parafilmed, then co-cultivated in the dark at 24° C.-26° C. for 2-3 days.

The co-cultivated PCIEs (pre-cultured immature embryo) are transferred to delay medium (CM4C+500 mg/L carbenicillin) in the dark. After seven days on the delay medium, the immature embryos are transferred to CM4C supplemented with 2 mM glyphosate (not Roundup® formulation) and 500 mg/L carbenicillin for selection for one week. Then calli are transferred to MMSO.2C+0.1 mM glyphosate (not Roundup formulation) +250 mg/L carbenicillin medium for 2 weeks under light for further selection. Embryogenic calli are transferred to a second regeneration medium MMSOC with lower glyphosate (not Roundup® formulation) concentration (0.02 mM) and 500 mg/L carbenicillin for plant regeneration. Embryogenic calli are transferred to fresh medium every two weeks. Regenerated plantlets are transferred to Sundae cups (Sweetheart Cup Company, Chicago, Ill.) containing the second regeneration medium for further growth and selection. The media components of MMSO and CM4C are described in Cheng et al. Plant Physiology 115: 971-980 (1997).

When the roots of transgenic plantlets are well established, the plants are transferred to soil for further evaluation.

Evaluation of Wheat Transgenic Plants for the Translation Repression System.

R0 plants are split when at least one secondary tiller had three leaves (and its own roots). The tillers are separated carefully, so that the secondary tiller is left with most of its own roots. Each transgenic plant is split to grow into two independent but genetically identical plants (“splits”). One split is treated with glyphosate and one saved as a non-sprayed control. Plants are treated with glyphosate at about the six leaf stage by means of a linear track sprayer set to deliver a 64 oz./A rate of glyphosate. Vegetative tolerance to the glyphosate is visually evaluated one week after spraying based on a scale of 0 to 5 (0=no observable/vegetative effect of glyphosate; 1=chlorosis observed; 2=advanced chlorosis, minor necrosis; 3=advanced chlorosis, moderate necrosis; 4=advanced chlorosis, severe necrosis; 5=no live tissue remaining). All R0 plants are isolated during anthesis with crusty bags to prevent outcrossing. Percent male sterility is estimated as the number of empty florets divided by the total number of florets (filled plus empty) in a head of all tillers.

Thirteen R0 transgenic wheat lines are identified that displayed 90-99% sterile when treated with glyphosate (Table 19). Their respective non-sprayed splits showed varying degrees of sterility that are within the range usually observed for regenerated non-transgenic plants (0-5%, Table 19). These sterile lines are from two different constructs (pMON42914 and pMON52008).

TABLE 19
Glyphosate induced male sterility in transgenic TRS wheat

| | | % Sterility- | % Sterility-No |
|---|---|---|---|
| Constructs | R0 Line# | RoundUp | RoundUp |
| pMON42914 | TA_S6545 | 90 | 0 |
| | TA_S7030 | 90 | 0 |
| | TA_S7040 | 90 | 0 |
| | TA_S7044 | 95 | 0 |
| | TA_S7045 | 99 | 0 |
| | TA_S7046 | 90 | 0 |
| | TA_S7281 | 90 | 0 |
| | TA_S7588 | 90 | 0 |
| | TA_S7594 | 90 | 5 |
| | TA-S7805 | 90 | 0 |
| | TA_S8990 | 90 | 0 |
| pMON52008 | TA-S10771 | 95 | 0 |
| | TA_S10772 | 95 | 0 |

### Example 11

### Transformed Rice with the MS2 Translation Repression System.

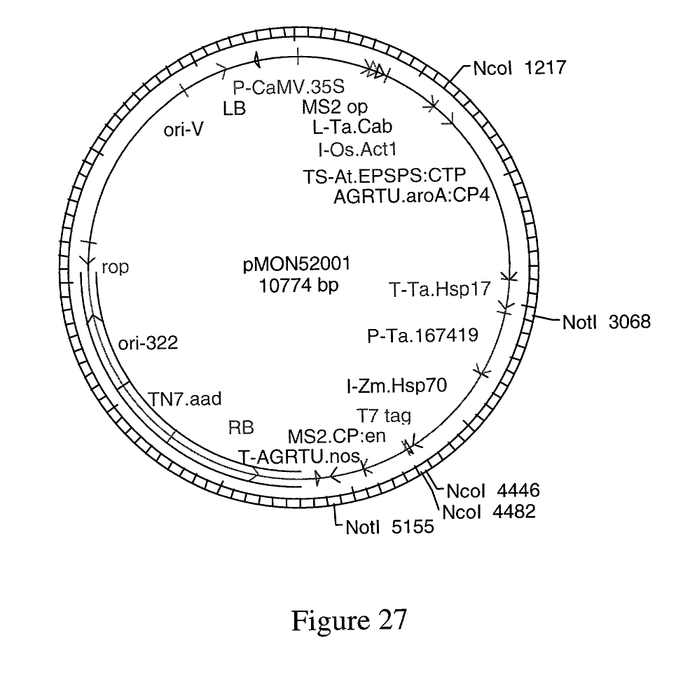

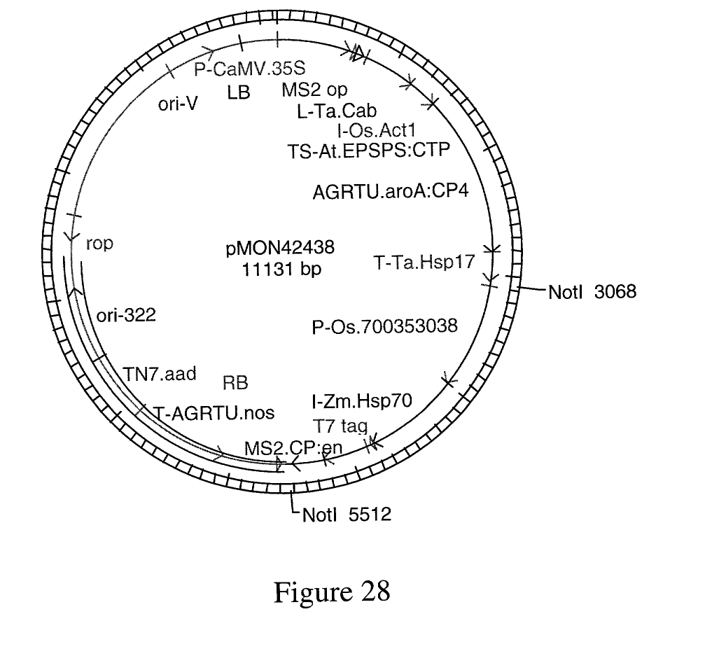

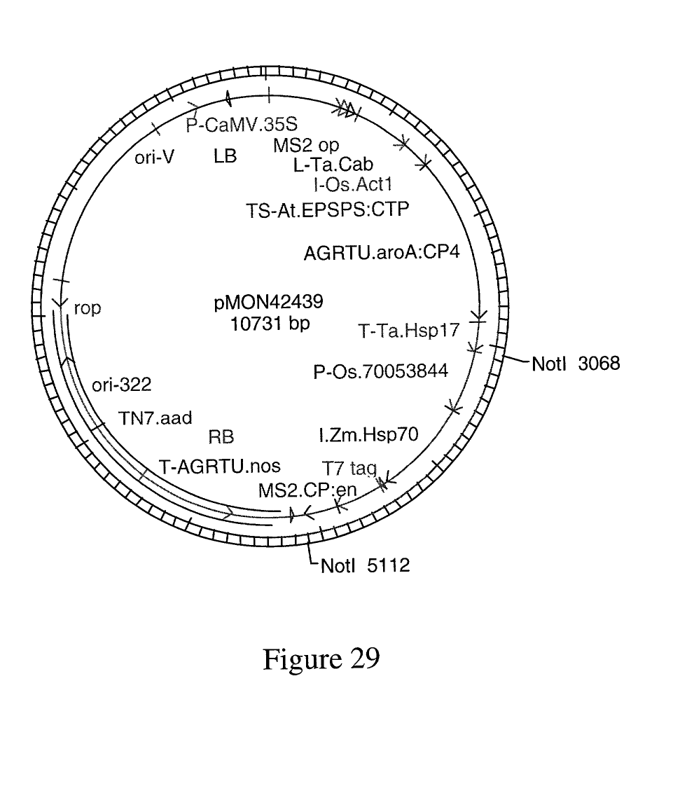

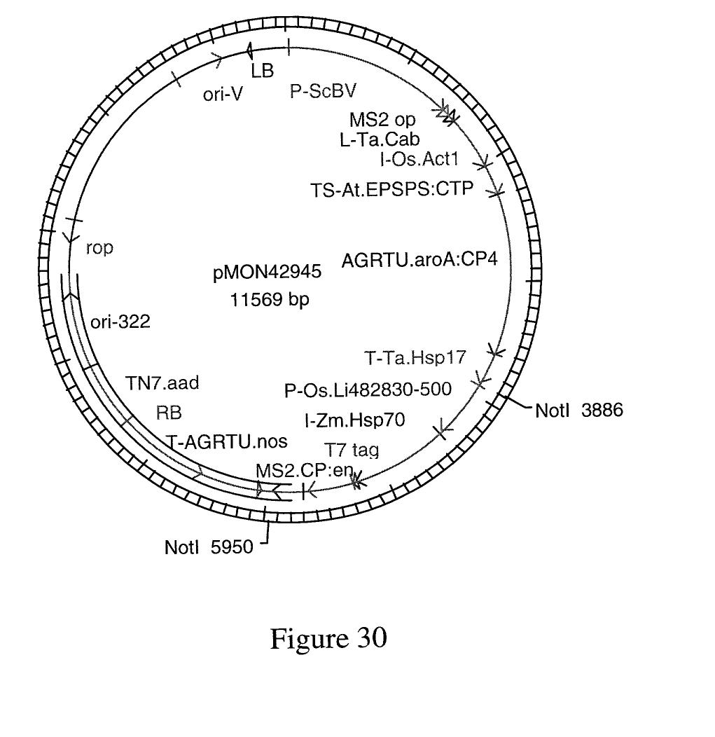

The anther-specific promoter P-Ta.1674-19 is amplified from wheat genomic DNA by Genome Walker™ as a HindIII/BglII fragment, digested with these two enzymes, and the fragment ligated into pMON42180 (FIG. 3) digested with the same enzymes, resulting in pMON52000. The plant expression vector pMON52001 (FIG. 27) is constructed by taking the Not I fragment containing of P-Ta. 1674-19/I-Zm.Hsp70/MS2. CP:en/T-AGRTU.nos from pMON52000 and ligating into pMON42916 (FIG. 7) digested with Not I. pMON42438 (FIG. 28) and pMON42439 (FIG. 29) are constructed by replacing the wheat P-Ta. 1674-19 promoter element of pMON52001 with corn anther-specific promoters P-700353038 and P-70053844, respectively. pMON42945 (FIG. 30) is constructed by ligating the Not I fragment consisting of P-L1482830/I-Zm.Hsp70/MS2.CP:en/T-AGRTU.nos into pMON42916. These promoter elements are isolated from wheat and corn anther DNA libraries by Genome Walker™. Rice variety M202 is transformed by Agrobacterium-mediated transformation. Freshly isolated immature embryos are inoculated with Agrobacterium Strain ABI containing pMON42438, pMON42439, pMON42945 or pMON52001 and co-incubated at room temperature for 15 minutes. The infected embryos are transferred to ½×MS CC1 medium (MS salts (Gibco BRL, Gaithersburg MD) 2.2 g/L, MS vitamins 1 mL/L, thiamine-HCl 0.5 mg/L, sucrose 20 g/L, glucose 10 g/L, L-proline 115 mg/L, 2,4-D (0.1 mg/mL stock) 20 mL/L, picloram (1 mg/mL stock) 2.2 mL/L) and kept in dark for 1 day, then transferred to MS Delay medium (MS salts 4.4 g/L, MS vitamins 1 mL/L, sucrose 20 g/L, glutamine 0.5 g/L, MgCl 0.75 g/L, casein hydrolysate 0.1 g/L, 2,4-D (0.1 mg/L stock) 20 mg, pH 5.8, Phytogel 2 g/L, picloram (1 mg/mL stock) 2.2 mL/L, carbenicillin 500 mg/L (40 ug/mL stock) 12.5 mL/L, silver nitrate (2 mg/mL stock) 1.7 mL/L), and incubated for 7 days in the dark at 23° C. to 27° C. For selection, the callusing tissues are transferred in the following order to GLY 1 medium (MS salts 4.4 g/L, MS vitamins 1 mL/L, sucrose 20 g/L, glutamine 0.5 g/L, MgCl 0.75 g/L, casein hydrolysate 0.1 g/L, 2,4-D (0.1 mg/L stock) 20 mL, pH 5.8, Phytogel 2 g/L, carbenicillin 250 mg/L, picloram 2.2 mg/L, glyphosate (not Roundup®) 2.0 mM), for 7 days; GLY 2 medium (MS salts 4.4g/L, MS vitamins 1 mL/L, Thiamine HCl 1.0 mg/L, sucrose 20 g/L, glutamine 0.5 g/L, Magnesium chloride 0.75 g/L, , 2,4-D (0.1 mg/L stock) 20 mL, pH 5.8, Phytogel 2 g/L, carbenicillin 250 mg/L, picloram 2.2 mg/L, glyphosate (not Roundup®) 0.5 mM) for 7 days; GLY 3 medium (MS salts 4.4 g/L, MS vitamins 1.0 mL/L, sucrose 20 g/L, 2,4-D (0.1 mg/L stock) 2.0 mL, pH 5.8, Phytagel 2.5 g/L, abscisic acid (1.0 mg/mL stock) 52 μL/L, 2.5 N HCl 280 μL/L, glyphosate (not Roundup®) 0.1 mM, carbenicillin 250 mg/L), 14 days in the dark at 23° C. to 27° C. The calli are then transferred to GLY 4 medium (MS salts 4.4g/L, MS vitamins 1.0 mL/L, myo-inositol 0.1 g/L, sucrose 60 g/L, pH 5.8, Phytagel 2 g/L, carbenicillin 100 mg/L) for 14 days and transferred to plant tissue culture vessels containing GLY 5 medium (MS salts (Gibco) 4.4 g/L, MS vitamins 1.0 mL/L, myo-inositol 50 mg/L, sucrose 60 g/L, pH 5.8, Schweizer Hall 2 g/L, glyphosate (not Roundup®) 20 mM, carbenicillin 100 mg/L, rice aromatic amino acid stock (L-phenylalanine 16.52 mg/L, L-tyrosine 21.77 mg/L, L-tryptophan 20.42 mg/L) 1 mL/L) for 14-30 days with 16 hour light period/8 hour dark period, for regeneration. Regenerated plants are finally transferred to soil and grown in greenhouse for further evaluation. When at least two, but preferably 4-6, tillers from the transgenic rice plant develop, the tillers of each transgenic plant are split and potted separately to grow into independent but genetically identical plants under greenhouse conditions suitable for growth of rice. One tiller is treated with glyphosate and another saved as a non-sprayed control. Plants are treated with glyphosate at about 48 DAP (days after planting) or before panicle emergence by means of a linear track sprayer set to deliver a 64 oz./A rate of glyphosate. Vegetative tolerance to the glyphosate is visually evaluated 21 DAT (day after treatment) based on a scale of 0 to 5 (as discussed above). The first five panicles from both glyphosate-treated and untreated plants are bagged before anthesis to prevent pollination from adjacent plants. Male sterility is scored at maturity by counting the number of empty florets and dividing by the total number of florets (filled plus empty) in each panicle.

Six R0 transgenic rice lines are identified that are completely sterile when treated with glyphosate (Table 20). The respective non-glyphosate sprayed split plant of each transgenic line showed a low level of male sterility, but within the range usually observed for non-transgenic plants under the conditions of the test (5-10%, Table 20). The non-transgenic parental control plant (M202) is killed by the glyphosate treatment and showed normal fertility under the conditions of the test. These transgenic sterile lines are from four different constructs, pMON42945 (FIG. 30), pMON52001 (FIG. 27), pMON42439 (FIG. 29), and pMON42438 (FIG. 28). The transgenic plants are vegetatively glyphosate-tolerant and male glyphosate-sensitive due to the expression of the translation repression system.

TABLE 20
Glyphosate induced male sterility in TRS rice plants rated at 21 DAT

| | | % Sterility-glyphosate treated | | | | | | | | | | | |
|---|---|---|---|---|---|---|---|---|---|---|---|---|---|
| RO Line | | Pan | | | | | | % Sterility-no treatment | | | | | |
| # | pMON | 1 | Pan2 | Pan3 | Pan4 | Pan5 | Ave | Pan1 | Pan2 | Pan3 | Pan4 | Pan5 | Ave |
| 16576 | 42945 | 100 | 100 | 100 | 100 | 100 | 100 | 5 | 5 | 5 | 5 | 5 | 5 |
| 16577 | 42945 | 100 | NA | NA | NA | NA | 100 | 5 | 5 | 5 | 5 | 5 | 5 |
| 14350 | 52001 | 100 | 100 | 100 | 100 | 100 | 100 | 10 | 5 | 5 | 0 | 5 | 5 |
| 14404 | 42439 | 100 | 100 | 100 | 100 | 100 | 100 | 5 | 5 | 5 | 30 | 5 | 10 |
| 14422 | 42439 | 100 | 100 | 100 | 100 | 100 | 100 | 5 | 0 | 10 | 15 | 20 | 10 |
| 14383 | 42438 | 100 | 100 | 100 | 100 | 100 | 100 | 5 | 5 | 10 | 10 | 10 | 8 |
| M202 | none | dead | dead | dead | dead | dead | dead | 10 | 10 | 5 | 10 | 5 | 8 |

Pan = panicle, 5 panicles from each plant are scored for fertility
NA = not available

Having illustrated and described the principles of the present invention, it should be apparent to persons skilled in the art that the invention can be modified in arrangement and detail without departing from such principles. We claim all modifications that are within the spirit and scope of the appended claims.

All publications and published patent documents cited in this specification are incorporated herein by reference to the same extent as if each individual publication or patent application is specifically and individually indicated to be incorporated by reference.

We claim:

1. A recombinant nucleic acid construct comprising a first transcriptional unit that comprises:
a 5′ untranslated region comprising a first promoter that is functional in a cell of a plant and at least one operator for binding of an RNA-binding protein,
a polypeptide-encoding DNA sequence that is expressed under the control of the promoter, and
a first 3′ non-translated region comprising a polyadenylation site operably linked to the polypeptide-encoding DNA sequence,
wherein transcription of the first transcriptional unit in a cell of the plant produces an mRNA comprising said at least one operator, and binding of the RNA-binding protein to said at least one operator modulates translation of the mRNA

2. The nucleic acid construct of claim 1 further comprising a second transcriptional unit that comprises:
a second promoter that is functional in the cell of the plant;
a DNA sequence that encodes the RNA-binding protein; and
a second 3′ non-translated region comprising a polyadenylation site operably linked to the DNA sequence that encodes the RNA-binding protein.

3. The nucleic acid construct of claim 2 wherein the second promoter is expressed selectively in a tissue of the plant such that translation of the polypeptide-encoding DNA sequence is modulated in the tissue.

4. The nucleic acid construct of claim 3 wherein the tissue of the plant is a reproductive tissue.

5. The nucleic acid construct of claim 4 wherein the reproductive tissue is a male reproductive tissue.

6. The nucleic acid construct of claim 3 wherein the second promoter is preferentially expressed in a male reproductive tissue.

7. The nucleic acid construct of claim 6 wherein the second promoter is selected from the group consisting of Xy1 promoter, RA8 promoter, Ms45 promoter, SGB6 promoter, Tapl promoter, Osg6B promoter, Sta44 promoter, MS2 promoter, Zmg13 promoter, TA29 promoter, SLG promoter; SLR1 promoter, RST2 promoter, ZmC5 promoter, A3 promoter, A6 promoter, A9 promoter, YY1 promoter, YY2 promoter, ZmABP1 promoter, ZmABP2 promoter, brassica oleosin-like gene promoter, Antirrhinum DEFH125 gene promoter, LePro 1 promoter, MROS gene promoters, brassica polygalaturonase gene promoter, Lat52 promoter, Lat59 promoter, and 1,3-beta-glucanase gene promoter.

8. The nucleic acid construct of claim 6 wherein the polypeptide-encoding DNA sequence is a gene required for male fertility, such that expression of the RNA-binding polypeptide in the male reproductive tissue reduces translation of the polypeptide-encoding DNA sequence and thereby causes male sterility.

9. The nucleic acid construct of claim 4 wherein the reproductive tissue is a female reproductive tissue.

10. The nucleic acid construct of claim 9 wherein the second promoter is preferentially expressed in the female reproductive tissue.

11. The nucleic acid construct of claim 10 wherein the second promoter is selected from the group consisting of an S-locus specific glycoprotein gene promoter, P26 promoter, P19 promoter, B200i4-2 promoter, DefH9 promoter, cysteine-rich extensin-like protein gene promoter, orchid O39 promoter, orchid 0126 promoter, orchid O108 promoter, orchid O141 promoter, potato SK2 promoter, and rice RPC312 promoter.

12. The nucleic acid construct of claim 10 wherein the polypeptide-encoding DNA sequence is a gene required for female fertility, such that expression of the RNA-binding polypeptide in the female reproductive tissue reduces translation of the polypeptide-encoding DNA sequence and thereby causes female sterility.

13. The nucleic acid construct of claim 3 wherein the first promoter is a constitutive promoter.

14. The nucleic acid construct of claim 13 wherein the first promoter is selected from the group consisting of a cauliflower mosaic virus 19S promoter, a cauliflower mosaic virus 35S promoter, a figwort mosaic virus 35S promoter, a sugarcane bacilliform virus promoter, a commelina yellow mottle virus promoter, a small subunit of ribulose-1,5-bisphosphate carboxylase promoter, a rice cytosolic triosephosphate isomerase promoter, an adenine phosphoribosyltransferae promoter, a rice actin 1 promoter, a mannopine synthase promoter, an octopine synthase promoter, and a histone promoter.

15. The nucleic acid construct of claim 1 wherein the polypeptide-encoding DNA sequence is selected from the group consisting of an herbicide tolerance gene, an insecticidal protein gene, an antibiotic protein gene, a gene that affects plant growth, a gene that affects plant metabolism or development, and a pharmaceutical protein gene.

16. The nucleic acid construct of claim 1 wherein the first promoter is a constitutive promoter, the polypeptide-encoding DNA sequence encodes a polypeptide that confers tolerance to an herbicide to the plant, and the second promoter causes the RNA-binding protein to be selectively expressed in a reproductive tissue of the plant, such that a plant transformed with the nucleic acid construct is vegetatively tolerant to an application of the herbicide and the reproductive tissue is sensitive to the application of the herbicide.

17. The nucleic acid construct of claim 16 wherein the herbicide is selected from the group consisting of glyphosate, benzonitrile, glufosinate, imidazolinones, cyclohexanedione, and sulfonylureas.

18. The nucleic acid construct of claim 17 wherein the herbicide is glyphosate.

19. The nucleic acid construct of claim 17 wherein the polypeptide-encoding DNA sequence encodes a glyphosate-resistant EPSP synthase or a glyphosate degrading enzyme.

20. The nucleic acid construct of claim 19 wherein the glyphosate degrading enzyme is a glyphosate oxidoreductase.

21. The nucleic acid construct of claim 1 wherein the plant is selected from the group consisting of corn, wheat, rice, canola, oat, barley, alfalfa, carrot, cotton, oilseed rape, sugar beet, sunflower, soybean, tomato, cucumber and squash, trees, ornamental annual plants, and perennial bedding plants.

22. The nucleic acid construct of claim 1 wherein the operator is selected from the group consisting of an RNA bacteriophage coat protein operator and a yeast ribosomal protein operator.

23. The nucleic acid construct of claim 22 wherein the RNA bacteriophage coat protein operator is an MS2 phage coat protein operator or a Qβ phage coat protein operator.

24. The nucleic acid construct of claim 22 wherein the yeast ribosomal protein operator is an RPL32 RNA-binding protein operator.

25. The nucleic acid construct of claim 1 wherein the RNA-binding protein operator is located between 0 and 37 nucleotides, inclusive, 3′ to the 5′ end of the mRNA.

26. The nucleic acid construct of claim 25 wherein the RNA-binding protein operator is located between 4 and 19 nucleotides, inclusive, 3′ to the 5′ end of the mRNA.

27. The nucleic acid construct of claim 1 wherein the 5′ untranslated region comprises multiple operators for binding of an RNA-binding protein.

28. The nucleic acid construct of claim 27 wherein the multiple operators are in tandem array.

29. The nucleic acid construct of claim 28 wherein the operators are separated by introns.

30. The nucleic acid construct of claim 2 wherein the DNA sequence that encodes the RNA-binding protein is modified for enhanced plant expression.

31. The nucleic acid construct of claim 2 wherein the DNA sequence that encodes the RNA-binding protein encodes an RNA-binding protein selected from the group consisting of MS2 coat protein, Qβ coat protein, and RPL32 RNA-binding protein.

32. The nucleic acid construct of claim 2 wherein the DNA sequence that encodes the RNA-binding protein encodes an RNA-binding protein dimer.

33. A plant comprising a recombinant nucleic acid construct comprising a first transcriptional unit that comprises:
a 5′ untranslated region comprising a first promoter that is functional in the cell of a plant and at least one operator for binding of an RNA-binding protein,
a polypeptide-encoding DNA sequence that is expressed under the control of the promoter, and
a first 3′ non-translated region comprising a polyadenylation site operably linked to the polypeptide-encoding DNA sequence,
wherein transcription of the first transcriptional unit in the cell of the plant produces an mRNA comprising said at least one operator, and binding of the RNA-binding protein to said at least one operator modulates translation of the mRNA.

34. The plant of claim 33 further comprising a second transcriptional unit that comprises:
a second promoter that is functional in the cell of the plant;
a DNA sequence that encodes the RNA-binding protein; and
a second 3′ non-translated region comprising a polyadenylation site operably linked to the DNA sequence that encodes the RNA-binding protein.

35. The plant of claim 34 wherein the nucleic acid construct comprises the first and second transcriptional units.

36. The plant of claims 33-35 wherein the second promoter is expressed selectively in a tissue of the plant such that translation of the polypeptide-encoding DNA sequence is modulated in the tissue.

37. A method of controlling translation of a polypeptide-encoding DNA sequence in a plant comprising:
providing a plant comprising a recombinant nucleic acid construct that comprises: (1) a first transcriptional unit comprising a 5′ untranslated region comprising a first promoter that is functional in the plant and at least one operator for binding of an RNA-binding protein, a polypeptide-encoding DNA sequence that is expressed under the control of the promoter, and a first 3′ non-translated region comprising a polyadenylation site operably linked to the polypeptide-encoding DNA sequence, and (2) a second transcriptional unit comprising a second promoter that is functional in the cell of the plant, a DNA sequence that encodes the RNA-binding protein, and a second 3′ non-translated region comprising a polyadenylation site operably linked to the DNA sequence that encodes the RNA-binding protein;
transcribing the first transcriptional unit in the plant cell to produce an mRNA comprising said at least one operator sequence, and
transcribing and translating the second transcriptional unit to produce the RNA-binding protein in a cell of the plant, wherein binding of the RNA-binding protein to said at least one operator modulates translation of the mRNA.

38. A method of producing a hybrid seed comprising:
providing a pollen-producing male parent and a male-sterile female parent, the female parent comprising: (1) a first transcriptional unit comprising a 5′ untranslated region comprising a first promoter that is functional in the female parent and at least one operator for binding of an RNA-binding protein, a polypeptide-encoding DNA sequence that is expressed under the control of the promoter, and a first 3′ non-translated region comprising a polyadenylation site operably linked to the polypeptide-encoding DNA sequence, and (2) a second transcriptional unit comprising a second promoter that is expressed in a male reproductive tissue of the female parent, a DNA sequence that encodes the RNA-binding protein, and a second 3′ non-translated region comprising a polyadenylation site operably linked to the DNA sequence that encodes the RNA-binding protein; wherein expression of the RNA-binding polypeptide in the male reproductive tissue reduces translation of the polypeptide-encoding DNA sequence and thereby causes male sterility; and
fertilizing the female parent with pollen from the male parent, thereby producing hybrid seed.

39. A method of producing a hybrid seed comprising:
providing a pollen-producing male parent and a male-sterile female parent, the female parent comprising: (1) a first transcriptional unit comprising a 5′ untranslated region comprising a first promoter that is functional in the female parent and at least one operator for binding of an RNA-binding protein, an herbicide-tolerance gene that is transcribed under the control of the promoter, and a first 3′ non-translated region comprising a polyadenylation site operably linked to the polypeptide-encoding DNA sequence, and (2) a second transcriptional unit comprising a second promoter that is expressed in a male reproductive tissue of the female parent, a DNA sequence that encodes the RNA-binding protein, and a second 3′ non-translated region comprising a polyadenylation site operably linked to the DNA sequence that encodes the RNA-binding protein; wherein expression of the RNA-binding polypeptide in the male reproductive tissue reduces translation of the herbicide-tolerance gene in the male reproductive tissue;
applying an herbicide to the female parent, thereby rendering the female parent male sterile; and
fertilizing the female parent with pollen from the male parent, thereby producing hybrid seed.

40. The method of claim 39 wherein the male parent comprises a third transcriptional unit comprising a 5′ untranslated region comprising a third promoter that is functional in the male parent and at least one operator for binding of an RNA-binding protein, an herbicide-tolerance gene that is expressed under the control of the third promoter, and a first 3′ non-translated region comprising a polyadenylation site operably linked to the herbicide-tolerance gene, and (2) a fourth transcriptional unit comprising a fourth promoter that is expressed in a female reproductive tissue of the male parent, a DNA sequence that encodes the RNA-binding protein, and a fourth 3′ non-translated region comprising a polyadenylation site operably linked to the DNA sequence that encodes the RNA-binding protein; wherein expression of the RNA-binding polypeptide in the female reproductive tissue reduces translation of the herbicide-tolerance gene in the female reproductive tissue, the method further comprising the step of applying the herbicide to the male parent, thereby rendering the male parent female sterile.
